(19) United States
(12) Patent Application Publication
(10) Pub. No.: US 2007/0111201 A1
(43) Pub. Date: May 17, 2007

(54) Reverse transfection of cell arrays for structural and functional analyses of proteins

(76) Inventor: Benjamin Doranz, Narberth, PA (US)

Correspondence Address: COZEN O'CONNOR, P.C., 1900 MARKET STREET, PHILADELPHIA, PA, 19103-3508, US

(21) Appl. No.: 11/222,193

(22) Filed: Sep. 8, 2005

Related U.S. Application Data
(63) Continuation-in-part of application No. 10/476,297, filed on Jan. 27, 2004.
(60) Provisional application No. 60/608,579, filed on Sep. 10, 2004.
(60) Provisional application No. 60/635,040, filed on Dec. 8, 2004.
(60) Provisional application No. 60/287,335, filed on Apr. 30, 2001.

(51) Int. Cl. C12Q 1/70; C12Q 1/68; C12M 3/00

(52) U.S. Cl. 435/5; 435/6; 435/287.2

(57) ABSTRACT

The present invention relates to articles and methods for determining the function of genes, gene products, and nucleic acid products. The present invention also relates to identifying ligands and binding partners or proteins and nucleic acid products. The present invention also relates to methods and compositions related to reverse-transfection.

### CROSS REFERENCE TO RELATED APPLICATIONS

This application claims priority to U.S. Provisional Application Ser. No. 60/608,579, filed on Sep. 10, 2004 and U.S. Provisional Application Serial No., 60/635,040, filed on Dec. 8, 2004, and is a continuation-in-part of U.S. application Ser. No. 10/476,297, filed on Jan. 27, 2004, which is a national phase filing of PCT application No. PCT/US02/13432, filed on Apr. 30, 2002, which claims priority to U.S. Provisional Application 60/287,335, filed on Apr. 30, 2001 each of which is hereby incorporated by reference in its entirety.

### GOVERNMENT SUPPORT

This invention was made with U.S. Government support (NIH Grant No. GM65755) and the U.S. Government may therefore have certain rights in the invention.

### BACKGROUND OF THE INVENTION

G protein coupled receptors (GPCRs) are a large family of 7-transmembrane proteins responsible for cellular communication (reviewed in (Morris, et al. (1999), Physiol Rev, 79:1373-430)). GPCRs account for approximately 2% of human genes, and act as receptors for a diverse range of ligands that include neurotransmitters, hormones, ions, and amino acids. Approximately 30% of currently available pharmaceuticals modify GPCR function, making these receptors the largest group of drug targets today.

Chemokine receptors are an important class of GPCR, involved in the trafficking of a variety of cell types, including T-cells, macrophages, and hematopoietic stem cells (reviewed in (Lee, et al. (1999), J Leukoc Biol, 65:552-65, Moser, et al. (2004), Trends Immunol, 25:75-84)). Approximately twenty chemokine receptors have been identified, and many of them have become targets for treating immune disorders, including multiple sclerosis and asthma. CCR5 is a 352 amino acid chemokine receptor expressed on the surface of memory T-cells, macrophages, and immature dendritic cells, that is upregulated by proinflammatory cytokines, and that is coupled to the Gi class of heterotrimeric G proteins (reviewed in (Blanpain, et al. (2002), Receptors Channels, 8:19-31)). Upon binding its chemokine ligands (e.g. MIP-1α, MIP-1β, and RANTES), CCR5 triggers cellular migration via a number of cell signaling mechanisms, including stimulation of Ca⁺² release from intracellular stores. Because of its ability to regulate immune cell migration, CCR5 has become a target for controlling diseases with autoimmune components, including rheumatoid arthritis.

In addition to its physiological functions, CCR5 is the principal coreceptor for human immunodeficiency virus type 1 (HIV-1), facilitating HIV-1 Envelope (Env) binding and viral fusion to the host cell (reviewed in (Berger, et al. (1999), Annu. Rev. Immunol., 17:657-700, Doranz (2000), Emerging Therapeutic Targets, 4:423-437)). CCR5 is a particularly attractive target for the control of HIV-1 infection because the protein can be eliminated from the human proteome without apparent side effects. Homozygous individuals with a naturally occurring mutation of CCR5 (CCR5Δ32, approximately 10% allelic frequency in populations of European origin) do not express the receptor on cell surfaces due to a premature truncation of the protein, but exhibit no apparent deleterious health effects (Liu, et al. (1996), Cell, 86:367-377, Samson, et al. (1996), Nature, 382:722-725). Significantly, individuals homozygous for the CCR5A32 variant are almost completely resistant to HIV-1 infection via all routes of transmission (Liu, et al. (1996), Cell, 86:367-377, Samson, et al. (1996), Nature, 382:722-725), and heterozygotes demonstrate a 2-4 year delay in progression to AIDS (Dean, et al. (1996), Science, 273:1856-1862, Huang, et al. (1996), Nature Med., 2:1240-1243, Michael, et al. (1997), Nature Med., 3:338-340). Anti-CCR5 agents are likely to be the next class of HIV-1 therapeutics to reach patients.

The chemokine receptor CXCR4 is a widely-expressed GPCR that regulates the trafficking of lymphocytes and hematopoietic stem cells in adults (Aiuti, et al. (1997), J. Exp. Med., 185:111-120, Bleul, et al. (1996), Nature, 382:829-833) and B-cell lymphopoiesis, bone-marrow myelopoiesis, and organ vascularization during embryonic development (CXCR4 and SDF-1α knockout mice die perinatally) (Ma, et al. (1999), Immunity, 10:463-71, Nagasawa, et al. (1996), Nature, 382:635-638, Tachibana, et al. (1998), Nature, 393:591-594). Upon binding its cognate chemokine ligand SDF-1α, CXCR4 triggers cellular migration via a number of cell signaling mechanisms, including activation of PI3-kinase and MAP kinase cascades, inhibition of cAMP production, and stimulation of Ca⁺² release from intracellular stores (Bleul, et al. (1996), Nature, 382:829-833, Ganju, et al. (1998), J Biol Chem, 273:23169-75, Oberlin, et al. (1996), Nature, 382:833-835). Its recognized functions in cell development and trafficking, as well as its link to the pathogenesis of several important diseases, have made CXCR4 an important focus for human health research.

The first link between CXCR4 and human disease was its discovery as a coreceptor for HIV-1 (Feng, et al. (1996), Science, 272:872-877). Strains of HIV-1 that use CXCR4 as a coreceptor (T-tropic strains) are associated with a course of infection that progresses more rapidly to the development of AIDS and death (Miedema, et al. (1994), Immunol. Rev., 140:35-72). The need for therapeutics that prevent HIV-1 infection via CXCR4 is becoming increasingly simportant. Drugs which block utilization of the other major coreceptor, CCR5, and which are now in clinical trials, may exert selective pressure on the virus to evolve more virulent characteristics, possibly by utilizing CXCR4 (Biti, et al. (1997), Nature Med., 3:252-253, Lu, et al. (1997), Proc. Natl. Acad. Sci. USA, 94:6426-6431, Michael, et al. (1998), J. Virol., 72:6040-6047, O'Brien, et al. (1997), Lancet, 349:1219, Theodorou, et al. (1997), Lancet, 349:1219-1220). In addition to its role in HIV-1 infection, CXCR4 has been linked to the development and spread of malignancies. CXCR4 was demonstrated to be highly expressed in human breast cancer cells, malignant breast tumors, and metastases, while high levels of SDF-1α expression were observed in organs representing the first destinations of breast cancer metastasis (Muller, et al. (2001), Nature, 410:50-6). Furthermore, inhibition of SDF-1α/CXCR4 interaction impaired metastasis in vivo (Muller, et al. (2001), Nature, 410:50-6), suggesting that CXCR4 could be an important target in the treatment of breast cancer (Murphy (2001), N Engl J Med, 345:833-5). CXCR4 also regulates hematopoietic stem cell migration both during development and, in certain cell populations, in adults, making it a target for increasing stem cell recovery after peripheral blood stem cell transplantation (Aiuti, et al. (1997), J. Exp. Med., 185:111-120, Lee, et al. (1998), Stem Cells, 16:79-88). “Bone marrow transplants” are commonly used during treatment of certain types of cancer, lymphomas and leukemias, and the success of the procedure significantly affects patient morbidity and mortality (Liles, et al. (2003), Blood, 102:2728-30). Treating HIV infection, breast cancer, and enhancing the success of bone marrow transplants are three high priority areas of human health research in which CXCR4 is beginning to receive increasing attention. However, this receptor has proven to be among the most difficult to manipulate and apply to conventional therapeutic development programs.

Human immunodeficiency virus type 1 (HIV-1) is the etiologic agent of acquired immunodeficiency syndrome (AIDS). Over 60 million people have been infected by HIV-1, and in 2003 alone over three million people worldwide died from AIDS (UNAIDS Epidemic Update (2003)). Without a viable vaccine, these figures are only expected to grow. Significant new treatments for HIV-1 have been developed over the past few years and are clearly making a difference in the infection rates of some populations. Moreover, new HIV-1 treatments such as fusion inhibitors are now on the market. Nevertheless, these treatments remain expensive, difficult to tolerate, and are increasingly plagued by the emergence of multi-drug resistant viruses. Most importantly, the majority of infected individuals simply do not have access to these drugs.

Despite substantial efforts, vaccine development against HIV-1 has met with limited success to date (Moore (2002), Nature, 415:365-366, Nabel (2002), Vaccine, 20:1945-1947). The virus mutates dramatically during infection, and even small mutations can have major consequences on antigenic structure, viral tropism, and evasion of immune response. Limiting factors in the development of new vaccines are the ability to 1) identify better immunogens, 2) characterize the epitopes of broadly cross-reactive and neutralizing monoclonal antibodies, and 3) quickly and easily measure the effectiveness of an individual's immune response. Tools that can contribute to these efforts are needed for vaccines against HIV-1 as well as other infectious pathogens.

HIV-1 infects a cell using an outer coat protein called Envelope (Env), also known as gp160 (Doranz (2000), Emerging Therapeutic Targets, 4:423-437). Env has been a prime target in the development of vaccines and drugs against the virus because of its exposure on the face of the virus and the prominent immune response against it. Env is a large protein (160 kDa), with complex folds (5 variable loops and 5 constant regions), intricate linkages (8 disulfide bonds), extensive modifications (approximately 50% of the protein's weight is composed of carbohydrate), and a highly ordered quaternary structure (each functional unit consists of a trimer of intertwined gp160 subunits). Each gp160 protein is cleaved into two non-covalently associated subunits, gp120 which binds receptors and gp41 which is transmembrane anchored and mediates membrane fusion.

The practical implication of this complexity is that the study of HIV-1 Env is a slow and labor-intensive task. For example, in order to understand the functional implications of nucleotide changes in Env, each Env variant must be studied one-by-one. Most typically, each Env mutant is produced in mammalian cells through transfection or infection of culture dishes of cells and then analyzed using Western, infection, or receptor binding assays. Using selection methodologies, swarms of HIV-1 can be simultaneously selected for phenotypes of interest, which has resulted in many interesting variants of HIV-1 (Edinger, et al. (1997), Proceedings of the National Academy of Sciences, USA, 94:14742-14747, Endres, et al. (1996), Cell, 87:745-756, LaBranche, et al. (1994), J. Virol., 68:5509-5522). However, selection techniques typically result in the characterization of only a handful of variants, and the swarm must be continually re-generated, re-selected, and re-cloned in order to isolate additional variants. Even then, mutations can occur across the 9.2 kb genome, not just in an area of interest. Using this process, correlation of structure and function is usually limited to just a few clones. An efficient system for studying complex proteins such as Env in a high throughput basis and in the context of living mammalian cells could have a significant commercial and scientific impact.

Identifying amino acid residues that constitute binding sites on target proteins is an important and desirable strategy for rational drug design, particularly during lead compound optimization. Understanding the structural basis for a drug's interaction with a target has particular relevance to HIV-1 inhibitor design, since anti-viral drugs can induce compensating viral mutations that facilitate drug evasion. The emergence of drug resistance has been observed in response to every anti-HIV drug used to date (reviewed in (De Clercq (2002), Med Res Rev, 22:531-65), and strains of HIV-1 exhibiting resistance to first-generation CCR5 inhibitors have already been identified (Billick, et al. (2004), J Virol, 78:4134-44, Kuhmann, et al. (2004), J Virol, 78:2790-807, Trkola, et al. (2002), Proc Natl Acad Sci USA, 99:395-400). HIV-1 strains appear to evade CCR5 inhibitors by altering their molecular interactions with the coreceptor, rather than by switching coreceptors altogether (Kuhmann, et al. (2004), J Virol, 78:2790-807, Pastore, et al. (2004), J Virol, 78:7565-74, Trkola, et al. (2002), Proc Natl Acad Sci USA, 99:395-400). As a result, identifying the structural bases for CCR5 binding of 1) HIV-1 Env and 2) potential drugs will be important in understanding the mechanism of this resistance, and in the diagnosis and treatment of patients that harbor drug resistant strains. However, describing these molecular interactions has not proven an easy or rapid task.

The techniques of x-ray crystallography and NMR have previously enabled visualization of the precise molecular interactions between drugs and HIV-1 targets, for example with the protease inhibitors Viracept™ and Norvir™, allowing HIV-1 evasion mechanisms to be understood and better treated (Erickson, et al. (1990), Science, 249:527-33, Humblet, et al. (1993), Antiviral Res, 21:73-84, Kaldor, et al. (1997), J Med Chem, 40:3979-85, Navia, et al. (1989), Nature, 337:615-20, Wlodawer, et al. (1998), Annu. Rev. Biophys. Biomol. Struct., 27:249-284). In the case of CCR5 inhibitors, however, structural techniques such as crystallography and NMR are not easily applied to GPCRs such as CCR5 because of the difficulty of producing, purifying, and crystallizing integral membrane proteins (Loll (2003), J Struct Biol, 142:144-53, Nollert, et al. (2004), DDT: Targets, 3:2-4, Torres, et al. (2003), Trends Biochem Sci, 28:137-44). Instead, scientists usually rely on site-directed mutagenesis to understand which regions of GPCRs compose the binding sites for drugs, epitopes for antibodies, and active sites in functional regions of the molecule.

Site-directed mutagenesis involves the introduction of specified mutations into a protein at targeted regions hypothesized to be of relevance. When used for structural mapping, individual mutations must be introduced one by one, resulting in a series of mutant proteins. Each mutation is sequence-verified, prepared by plasmid purification, transfected into cells, and assayed for function (e.g. binding of a labeled compound, infection by a virus, signaling in response to a ligand, or inhibition of the same). This process results in a ‘map’ of the residues that contribute to a particular binding or functional site, with the detail of the map directly correlated to the number of mutations analyzed. The value of this strategy is significant, as demonstrated by the use of site-directed mutagenesis to identify a transmembrane helical binding site for the CCR5 inhibitor TAK-779, the first detailed structural data obtained for a drug interacting with this receptor (Dragic, et al. (2000), Proc. Natl. Acad. Sci. USA, 97:2639-2644). Similar mutational analyses of CCR5 and other chemokine receptors have been performed to identify sites involved in HIV-1 Env binding, HIV-1 Env fusion, chemokine binding, chemokine activation, and G protein coupling (Baik, et al. (1999), Virology, 259:267-273, Blanpain, et al. (1999), J. Biol. Chem., 274:34719-34727, Blanpain, et al. (1999), J. Biol. Chem., 274:18902-18908, Blanpain, et al. (1999), Blood, 94:1899-1905, Doranz, et al. (1997), J. Virol., 71:6305-6314, Edinger, et al. (1997), Proc. Natl. Acad. Sci. USA, 94:4005-4010, Lee, et al. (1999), J. Biol. Chem., 274:9617-9626, Rucker, et al. (1996), Cell, 87:437-446). The structural information derived from site-directed mutagenesis is not as precise as crystallography, but GPCR mapping studies have allowed the more rational design of inhibitors, the prediction of inhibitor effects, and, most importantly for GPCRs, correlation of protein structures with function (which cannot be predicted using structural data alone). As a result, the functional analysis of point mutations is currently the primary experimental support for the modeling of GPCRs during drug discovery, including the drug interaction models created for CCR5 (Huang, et al. (2000), Acta Pharmacol Sin, 21:521-8, Paterlini (2002), Biophys J, 83:3012-31).

Despite the value of this approach, the functional analysis of GPCR mutations and other protein mutations is a slow, laborious task. Each mutant clone must be individually transfected into living cells, and the resulting expressed proteins analyzed separately, for example for the CCR5 inhibitor TAK-779 (Dragic, et al. (2000), Proc. Natl. Acad. Sci. USA, 97:2639-2644). The work has had to be painstakingly repeated to construct maps for each additional CCR5 inhibitor (Billick, et al. (2004), J Virol, 78:4134-44, Huang, et al. (2000), Acta Pharmacol Sin, 21:521-8, Paterlini (2002), Biophys J, 83:3012-31, Tsamis, et al. (2003), J Virol, 77:5201-8).

While mutagenesis of genetic clones can be performed relatively easily using modern molecular biology, the functional characterization of their protein products in living cells can be labor intensive and time consuming, particularly for large libraries. The primary bottleneck in the high throughput analysis of large plasmid libraries is the time and effort required to transfect each clone into cells. Typically, DNA from each plasmid is individually mixed with a transfection reagent (often a lipid-based carrier) and added, plasmid-by-plasmid, to pre-cultured cells. Alternatively, mixed pools of a cDNA library (or fractions thereof) can be transfected into cells and screened for the presence of a particular clone. This latter approach has had significant impact in identifying genes of novel function, for instance the initial identification of the chemokine receptor CXCR4 as a coreceptor for HIV-1 (Feng, et al. (1996), Science, 272:872-877). However, the approach also requires significant time and effort, and in many, if not most, cases, fails to identify genes of interest. Individual clones represent a vanishingly small fraction of the entire pool, making the most important mutations difficult to isolate. There is a clear need for the development of techniques and tools that simplify the transfection procedure in order to facilitate reliable high throughput screening of large nucleotide libraries.

### SUMMARY OF INVENTION

In some embodiments, the present invention provides microarrays comprising an array of nucleic acid molecules and a transfection reagent wherein the nucleic acid molecules and transfection reagent are frozen.

In some embodiments, the microarrays are free of cells.

In some embodiments, the present invention provides microarrays comprising an array of nucleic acid molecules and a transfection reagent wherein the array of nucleic acid molecules comprises polymorphisms of a gene and wherein the transfection reagent and nucleic acid molecules are frozen or dried onto the plate or microarray.

In some embodiments, the present invention provides microarrays comprising an array of nucleic acid molecules and a transfection reagent wherein the nucleic acid molecules encode at least one sensor and, optionally, at least one reporter, wherein the nucleic acid molecules and transfection reagent are frozen or dried on to the plate or microarray.

In some embodiments, the sensor of the present invention comprise an antibody or antibody-like molecule.

In some embodiments, at least one sensor is expressed on the surface of a cell.

In some embodiments, the reporter that is used is either a molecule or a method such, but not limited to FRET, BRET, or protein complementation.

The present invention also provides methods of preparing a microarray for reverse-transfection, comprising an array of nucleic acid molecules comprising depositing a composition comprising a nucleic acid molecule and a transfection reagent into a well of a microarray; and freezing the microarray.

In some embodiments, the nucleic acid molecule encodes at least part of a virus.

In some embodiments, the nucleic acid molecule encodes for a protein or a transcription element.

In some embodiments, the nucleic acid molecule comprises a promoter and is operatively linked to a reporter gene.

In some embodiments, the protein that is produced using the methods described herein is fixed onto a surface. In some embodiments, the protein is specifically attached to a surface using a tag on the protein.

The present invention also provides methods of introducing a nucleic acid molecule into a cell comprising thawing a microarray comprising a composition comprising a nucleic acid molecule; and contacting a well or spot of the microarray with the cell under appropriate conditions for entry of the nucleic acid molecule into the cell. In some embodiments, the cells are viable after entry of the nucleic acid molecule and are recovered for further propoagation.

The present invention also provides, in some embodiments, methods of mapping a function of a nucleic acid molecule product comprising contacting a composition comprising an array of nucleic acid molecules with a cell under appropriate conditions for entry of the nucleic acid molecule into the cell; and measuring a function of the nucleic acid molecule product, wherein a change in the function of the protein indicates that the residue that is mutated is involved in the function and is mapped to that region of the protein. In some embodiments, the present invention provides methods of preparing an array of viable cells in a microarray comprising thawing a frozen microarray comprising an array of nucleic acid molecules; and contacting a well or spot in the microarray with the cells. In some embodiments, the cells are transfected with the nucleic acid molecule.

In some embodiments, the present invention provides an array of virus, wherein the array is arranged on a slide surface or in a multi-well plate.

In some embodiments, the present invention provides frozen microarrays comprising an array of cells, a nucleic acid molecule, a transfection reagent, or combinations thereof. In some embodiments, the frozen microarrays comprise nucleic acid molecules that encodes at least one sensor and, optionally, at least one reporter.

In some embodiments, the present invention provides methods of selecting a vaccine candidate protein comprising contacting a composition comprising an array of nucleic acid molecules which encodes variants of the protein with a cell under appropriate conditions for entry of the nucleic acid molecule into the cell and expression of the protein on the cell surface; and measuring the binding of the protein to a first antibody specific for the protein and, optionally, to a second antibody, wherein the second antibody is non-neutralizing binding antibody wherein a protein that binds to the first antibody, but not to the second antibody is selected as a vaccine candidate. In some embodiments, the first antibody has desirable properties. By “desirable properties” it is meant that the antibody can bind to and neutralize the protein or a pathogen. In some embodiments, the second antibody has undesirable properties in that it can only bind to specific strains of a protein or a pathogen's protein. In some embodiments, the selected vaccine candidate is mutated and steps described herein are repeated to select an improved vaccine candidate, wherein the array encodes for variants of the selected candidate. In some embodiments, the first antibody is a neutralizing antibody. In some embodiments the second antibody is a non-neutralizing antibody. In some embodiments, only one antibody is used to select a protein that either binds better to a specific antibody or binds less to a non-specific antibody without comparing the binding of the protein to the different types of antibodies

The present invention also provides, in some embodiments, methods of identifying a drug-resistant mutant of a virus comprising contacting a composition comprising an array of nucleic acid molecules which encodes the virus or portion of the virus, with a cell under appropriate conditions for entry of the nucleic acid molecule into the cell and production of the virus; contacting the cell with an anti-viral drug; and measuring the growth of the virus, wherein an increase in growth as compared to the normal virus indicates that the mutant virus is a drug-resistant virus.

In some embodiments, the present invention provides, methods of producing a virus comprising contacting a composition comprising an array of nucleic acid molecules which encodes one or more genes from the virus with a cell under appropriate conditions for entry of the nucleic acid molecule into the cell; and contacting the cell with one or more nucleic acid molecules that encode one or more genes sufficient to produce the rest of the virus; and measuring the growth or infection of the virus.

In some embodiments, the present invention provides methods for detecting a pathogen in a sample comprising contacting the sample with an array of cells comprising ligands for the pathogens, wherein the ligands comprise a signaling mechanism that is activated upon binding of the pathogens to the ligands; and detecting a signal, whereby detection of the signal indicates the presence of the pathogen.

In some embodiments, the present invention provides methods for detecting a protein in a sample comprising contacting the sample with an array of cells comprising binding partners for the protein, wherein the binding partners comprise a signaling mechanism that is activated upon binding of the protein to the binding partners; and detecting a signal, whereby detection of the signal indicates the presence of the protein.

### BRIEF DESCRIPTION OF DRAWINGS

FIG. 1. A. The number of clones containing the indicated number of nucleotide changes introduced by random mutagenesis (Clontech Diversify kit) is indicated for both JRFL and MN Envs, as determined by sequence analysis of 500-700 bp reading frames. B. Overlapping PCR was used to introduce random mutations at an individual codon within the JRFL Env V3 loop (His306). The general strategy of mutagenesis is shown, highlighting the four primers and three PCR products generated. C. Two independent rounds of His306 mutation were conducted, and twenty-one clones were sequenced. The resulting codons are indicated for each round, as well as a separate round of mutagenesis on Gly538 (part of the HR1 helix in gp41) (last column of table). Seven different codons were involved in the mutagenesis of His306 to Arg, Ser, and Gly (2-3 clones of each).

FIG. 2. An array of living cells reverse transfected with CCR5-GFP. A. HEK-293 cells were reverse transfected with a plasmid encoding a CCR5-GFP fusion protein. Each well contains approximately 100,000 cells. The last three wells received cells but not DNA, so exhibit no fluorescence. After 24 h expression, live cells were imaged using an Alpha Array 7500i at 7 um resolution. A single well from the plate is enlarged in B. and individual cells are visible when the image is enlarged to its maximum resolution (C.). Transfection efficiency was estimated at 70-90%. Results demonstrate the ability to prepare and analyze an entire array of reverse transfected cells containing any desired plasmid in each well.

FIG. 3. Cell array prepared in a 384-well plate. The DNA-Lipofectamine complex was frozen while in solution, rather than dried, and cells expressing CCR5-GFP were visualized 2-days post-transfection. DNA was not added in every other well in the bottom row.

FIG. 4. DNA plasmids encoding the fluorescent proteins GFP or dsRED and mixed with Lipofectamine 2000 (as in FIG. 2) were arrayed on a poly-lysine-coated glass slide and then overlayed with HEK-293 cells. Cells were fixed at 48 hours post-transfection with 4% paraformaldehyde for 10 minutes. Fixed arrays were visualized with A. an AlphaArray 7000 imager, and B. and C. a Nikon inverted microscope under epifluorescent illumination. Each spot (as shown in B and C) consists of approximately 200 cells. Under white light, a confluent monolayer of cells across the entire slide is seen.

FIG. 5. A. Plasmid DNA encoding the indicated fluorescent proteins was prepared using three different kits (1 maxiprep and 2 different miniprep kits), arrayed in a 96-well microplate in the presence of the indicated additives, frozen without drying, and reverse transfected into HEK-293 cells. The plate was stored at −80° C. for 4 days prior to adding cells, which were allowed to express the proteins for 24 hours prior to imaging with an AlphaArray 7500.

FIG. 6. A. An array of cells expressing the fluorescent reporter protein dsRED was visualized using an AlphaArray 7000 over the course of 48 hours. Cells were alive during the entire time course, enabling the same cells to be repeatedly imaged while protein expression occurred. Quantitative expression levels of B. red fluorescent protein (dsRED) and C. green fluorescent protein (GFP) reporter proteins are graphed. Both proteins were expressed using identical CMV promoters. Each line represents an entire set of images measured over time, and each point on each line is the average of 48 spots on the cell array, as pictured in panel A. For each reporter, two arrays were treated with the promoter-enhancer sodium butyrate (NaB), added at t=20 h.

FIG. 7. Detection of gp160 on the cell surface by immunofluorescence. A. Fixed cells, after expression of an MN Env Array. Expression was detected using anti-V5 antibody and a Cy3 secondary antibody. B. Raw fluorescence values for V5 detection are shown. C. Details of individual clones are shown, highlighting some of the interesting clones identified in the Env array. D. “Bar-code” summary of multiple characteristics for the entire prototype MN Env Array. Three horizontal sequences represent three different antibodies. Each sequence is composed of vertical bars, representing the reactivity of each clone with that antibody. Bars are color-coded to represent clones with approximate activity >20% (green), 10-20% (yellow) or <10% (red) of wild type activity.

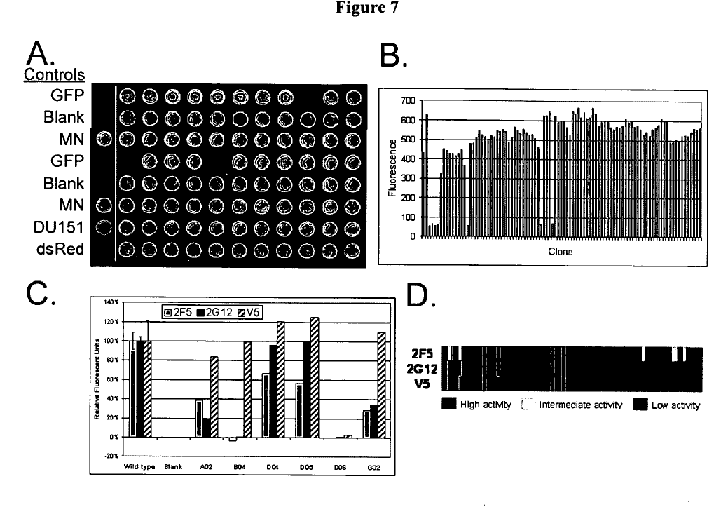

FIG. 8. A library of CCR5 mutations was prepared for reverse-transfection in a 96-well microplate. Each clone of the library contains approximately 1-2 random amino acid changes compared to wild type CCR5. The mutations were introduced in either the 3′ half of the gene or the 5′ half of the gene, as indicated. Each clone also contains an N-terminal HA epitope tag, and a C-terminal V5 epitope tag. Detection of HA on the surface of cells and V5 within cells by immunofluorescence measures the ability of each mutant to traffic to the cell surface and to be fully translated, respectively. The CCR5 mutation library was also fixed with paraformaldehyde and stained with the CCR5 monoclonal antibodies 2D7 and 45523, which both recognize conformational epitopes on the extracellular face of CCR5. Each Mutation Array is constructed with numerous controls, including parental (unmutated) HA-CCR5-V5 (‘CCR5 maxi’), and dsRED fluorescent proteins.

FIG. 9. Cell arrays can be used to detect multiple types of proteins. A. HEK-293 cells were reverse-transfected with a plasmid encoding a CCR5-GFP fusion protein. Each well contains approximately 100,000 cells. The last three wells received cells but not DNA, so exhibit no fluorescence. After 24 h expression, live cells were imaged using an Alpha Array 7000i at 7 um resolution. Results demonstrate the ability to prepare and image an entire array of reverse-transfected cells containing any desired plasmid in each well. B. Cells in quadruplicate wells of a microplate were reverse-transfected with one of eight plasmids encoding HIV-1 Env (strain MN), cell receptors (CXCR4, CCR5, and CD4), fluorescent proteins (GFP, dsRed), or vector alone (pcDNA). Cells transfected with fluorescent protein constructs were imaged directly. Other cells were fixed and stained using immunofluorescent targeting of a V5 epitope tag (Cy3 fluorophore). The image shows the same array imaged with green (left) and red (right) filters.

FIG. 10. Plasmids expressing the indicated proteins (GFP or dsRED) were prepared for reverse-transfection by drying in 96-well microplate wells. The indicated cell lines were added to the wells and fluorescence was visualized in the live cells post-transfection.

FIG. 11. Measurement of cell array variability and reproducibility. A. Cell arrays were constructed using dilutions of GFP and dsRED plasmids, as indicated. Transfected wells received 0.2 ug of total dsRED+GFP plasmids. The image shows the same array imaged with red (left) and green (right) filters. Data was collected on an AlphaArray 7000i, and quantified using AlphaEase software. B. Results from quantitative analyses are shown. The identical experiment was conducted on two different days (#1 and #2) and results are superimposed on the same axes for comparison.

FIG. 12. Detection of gp160 function in Env Array format by infection and neutralization. A. A DU151 Env Array was used to produce luciferase reporter virus, and these viruses were then used to infect U87-CCR5-CD4 cells, in duplicate. Luciferase activity in the target cells was measured two days post-infection. B. “Bar-code” summary of data from the DU151 Env Array. Each horizontal sequence represents a different function (infection, inhibition of infection, or antibody immunofluorescence), and each vertical bar identifies an individual clone. Bars are color-coded to represent clones with approximate activity >1,000 RLU (green), 200-1,000 RLU (yellow) or <200 RLU (red). C and D. Details of individual clones are shown, highlighting some clones of interest identified in the DU 151 Env Array.

FIG. 13. U87 cells stably expressing CD4 and reverse-transfected with a CCR5 mutation library were infected with an HIV-1 (strain ADA) reporter virus that expresses luciferase upon successful entry into a cell. Two days post-infection, cells were lysed and assayed for luciferase activity.

FIG. 14. CCR5 Analysis. A. Results from the analysis of a CCR5 Mutation Array are summarized. Each horizontal sequence represents a different test (e.g. function or binding partner) of the Mutation Array, and each vertical bar within the sequence identifies clones that traffic to the surface (HA), are fully translated (V5), contribute to the epitopes of the MAbs 2D7 and 45523, and support HIV-1 coreceptor activity (HIV-1). Bars are color-coded to represent clones with activity >40% (green), 10-40% (yellow) or <10% (red) of wild type activity. B. Details of individual clones are shown, highlighting some of the interesting clones identified in the CCR5 Mutation Array.

FIG. 15. High content analysis. Selected mutants of CCR5 expressed in HEK-293 cells were imaged with a Nikon Eclipse microscope (10× magnification) to illustrate the effects of some mutations on the trafficking patterns of CCR5 within cells. Parental CCR5 normally traffics to the cell surface, resulting in plasma-membrane staining of each cell (left panel). In contrast, the two mutant clones shown (P-X 39 and P-X 22) demonstrated unusual trafficking patterns. All clones are detected using immunofluorescent detection of a C-terminal V5 epitope tag on CCR5, demonstrating full-length translation of the clones.

FIG. 16. The H07 clone was identified from CCR5 Mutation Array screening and was further characterized and confirmed in the assays described here. The H07 clone performed comparably to wild type with respect to HA, V5, 45523, and 2D7 MAb immunofluorescence (quantified values of immunofluorescence staining, left panel). Surface staining at wild type levels was also confirmed by flow cytometry using HA, 45523, and 2D7 antibodies. When an R5 HIV reporter virus (DU151) carrying a luciferase gene was used for infection assays, H07 displayed impaired ability to function as a coreceptor. Infections were performed in 96-wells with the indicated amounts of virus added to 100 ul of supernatant on U87 cells expressing human CD4 and the indicated CCR5 (wild type, H07, or no CCR5).

FIG. 17. Overview of the expressed, self-assembling protein array. The expressed, self-assembling protein array is a self-assembling, self-reporting protein array capable of detecting pathogens and antibodies. In its final form, the array may be assembled in an array-within-array format. Each well of the microplate will contain an array of cells (cells are added just before use), and each spot in the array will be composed of a group of 200-1,000 cells. Each cell within a defined spot will display a sensor with a defined pathogen- or antibody-recognition element. Each sensor may also contain a sensitive mechanism for reporting target binding.

FIG. 18. Applications of the expressed, self-assembling protein array: pathogen detection and antibody detection. The sensors for each are nearly identical in concept and construction except that the pathogen detector has a modular Fab recognition determinant and the antibody sensor has an antigen of interest fused to the transmembrane domain and intracellular regions of PDGFR. Dimerization of the sensors, by multimeric pathogens or bivalent antibodies, respectively, will induce PDGFR signaling that can be detected by aequorin luminescence, FRET, BRET, or complementation.

### DETAILED DESCRIPTION

It is to be understood that the figures and descriptions of the present invention have been simplified to illustrate elements that are relevant for a clear understanding of the present invention, while eliminating, for purposes of clarity, many other elements found in gene mutation, transfection, and protein structural analyses. While this invention has been disclosed with reference to specific embodiments, it is apparent that other embodiments and variations of this invention may be devised by others skilled in the art without departing from the true spirit and scope of the invention. Those of ordinary skill in the art will recognize that other elements are desirable and/or required in order to implement the present invention. However, because they do not facilitate a better understanding of the present invention, a discussion of such elements is not provided herein. The disclosures of each and every patent, patent application, and publication cited herein are hereby incorporated herein by reference in their entirety.

By automating the transfection process using a technique termed “reverse transfection”, the transfection bottleneck involved in cellular analyses can be avoided. Reverse transfection allows plasmid DNA to be placed onto a surface prior to the addition of living cells, permitting automation of transfection, the most time-consuming part of cell preparation. To perform reverse transfection, DNA is first spotted onto a surface in complex with, e.g., a lipid-based transfectant, and then overlaid with living cells which subsequently take it up. The drying of the DNA onto the substrate prior to the addition of living cells confers two major benefits for the screening of plasmid libraries: 1) arrayed DNA ready for transfection can be stored for long periods of time prior to the addition of cells, thus automating the transfection steps, and 2) DNA can be selectively arranged on the substrate, ensuring location-specific expression of gene products. This has allowed the production of “cell arrays”, where the expression of specific gene products can be pre-determined in cells that adhere over appropriate DNA spots. The addition of transfection reagent and DNA to microplate wells prior to the addition of cells is a protocol described by Invitrogen (Carlsbad, Calif., Lipofectamine 2000 product notes)(Dalby, et al. (2004), Methods, 33:95-103)

A publication in 2001 describing one reverse transfection process used the technique to screen 192 different cDNAs for novel functions (Ziauddin, et al. (2001), Nature, 411:107-110), and has subsequently found diverse application for the analysis of RNAi libraries (Kumar, et al. (2003), Genome Res, 13:2333-40, Mousses, et al. (2003), Genome Res, 13:2341-7, Silva, et al. (2004), Proc Natl Acad Sci USA, 101:6548-52, Yoshikawa, et al. (2004), J Control Release, 96:227-32), monitoring of kinase signaling pathways (Webb, et al. (2003), J Biomol Screen, 8:620-3), screening of GPCR functions (Mishina, et al. (2004), J Biomol Screen, 9:196-207), and intracellular localization of proteins (Palmer, et al. (2004), Comparative and Functional Genomics, 5:342-353). Additional manuscripts have reported modifications and optimization of the reverse transfection process (Baghdoyan, et al. (2004), Nucleic Acids Res, 32:e77, Chang, et al. (2004), Nucleic Acids Res, 32:e33, Kato, et al. (2003), Biotechniques, 35:1014-8, 1020-1, Koichi, et al. (2003), Biotechniques, 35:1014-1018, Segura, et al. (2002), Bioconjug Chem, 13:621-9, Segura, et al. (2003), J Control Release, 93:69-84, Yoshikawa, et al. (2004), J Control Release, 96:227-32, Zhu, et al. (2002), J Proteome Res, 1:559-62). Reverse transfection is also described in U.S. Pat. No. 6,544,790 and U.S. Patent Application 20040132008, each of which is herein incorporated by reference in their entirety.

Reverse transfection offers advantages in time and scale compared with either standard transfection of individual plasmids, one-by-one at the time of use, or the selection and maintenance of stable cell lines that constitutively express the desired gene. These conventional techniques are impractical for more than a few dozen clones, and if performed at all are not easily repeated. By contrast, reverse transfection allows hundreds of clones to be interrogated simultaneously with little variation from one array to the next.

Arrays of adenovirus in microplates, each expressing different cDNAs, has been described (Michiels, et al. (2002), Nat Biotechnol, 20:1154-7).

The present invention also relates to an array of protein fixed on a surface. Fixation of proteins on a surface can be accomplished using any method of chemical including, but not limited to, paraformaldehyde, formaldehyde, methanol, glutaraldehyde, OsO4, or a number of other protein and/or lipid fixatives (Ausubel, et al. (2001), Current Protocols in Molecular Biology).

The present invention also relates to an improved means for detecting pathogens and antibodies in a sample.

Conventional diagnostic techniques used to detect pathogens and antibodies include ELISA, PCR, immunofluorescence (IF), and cell culture. Each of these techniques has wide utility, but also well-described limitations. Protein arrays have emerged as a competitive alternative to conventional ELISA assays, capable of detecting the composition of samples with the additional benefits of miniaturization and multiplexed detection (reviewed in (Nielsen, et al. (2004), J Immunol Methods, 290:107-20, Phelan, et al. (2003), Proteomics, 3:2123-34)). Protein arrays typically comprise antibodies (or other “capture” proteins) immobilized (or “spotted”) onto a surface. Binding of a target to each spot is usually detected either by a) using a labeled detection protein (often biotinylated versions of the capture antibodies), or b) labeling the sample itself prior to binding to the array (e.g. fluorescently labeling all of the proteins in the sample and then detecting the fraction that binds to the array). Fixed protein arrays have the power to screen samples for multiple targets simultaneously. However their construction requires complex protein extraction and purification steps, and immobilization of functional proteins to a substrate. Protein arrays are inherently unstable and difficult to construct using membrane proteins (Fang, et al. (2002), J Am Chem Soc, 124:2394-2395), which usually require a lipid environment to maintain their structure.

Protein arrays are limited because protein production is expensive and time-consuming. Each capture protein “spot” featured on the array must be constructed using recombinant proteins, monoclonal antibodies, and/or polyclonal serum. Production and purification of recombinant proteins and monoclonal antibodies is cumbersome, expensive, and logistically difficult as the complexity of the array grows, while polyclonal antibodies take months to produce, are not renewable once depleted, and must be re-validated for each new batch.

Protein arrays are also limited because proteins are not stable after spotting. The stability of proteins on filters and glass slides is poor. Current protein arrays are a trade-off in stability, conformational preservation, and background binding, usually resulting in poor sensitivity and high variability.

Protein arrays are also limited because detection of target binding is cumbersome and insensitive. Non-specifically labeling an entire test sample prior to array binding (e.g. labeling an entire serum sample with fluorescent tags) is time-consuming and impractical for detecting many targets. This approach results in high background labeling, and reduced specificity and sensitivity. Detection using a labeled secondary antibody is limited by the number of spots on the array (detection is performed with a mixture of labeled antibodies directly proportional to the number of spots on the array), which compromises sensitivity.

The present invention is also related to the production and use of cell-based biosensors. Cell-based biosensors exploit the sensitivity of endogenous cell signaling machinery. The inherent fidelity, simplicity, and speed of cellular signaling pathways are increasingly being exploited for diagnostic and screening assays (Ateya, et al. (2005), Anal Chem, 77:1290-4, Bechor, et al. (2002), J Biotechnol, 94:125-32, Haruyama (2003), Adv Drug Deliv Rev, 55:393-401, Kamei, et al. (2003), Biotechnol Lett, 25:321-5, Park, et al. (2003), Biotechnol Prog, 19:243-53). Many such ‘biosensors’ generate a signal by catalyzing a substrate, but do not require labeling of the targets of interest, hence are considered “label-free”. As an example, CANARY (Cellular Analysis and Notification of Antigen Risks and Yields) is a cell-based biosensor used for label-free detection of pathogens (Rider, et al. (2003), Science, 301:213-5). In CANARY, pathogen-specific B-cell lines stably expressing cytosolic aequorin (a calcium-sensitive bioluminescent protein) are used as biosensors. Dimerization of surface immunoglobulin (B-cell receptors, BCRS) induces a cascade of intracellular signaling events culminating in rapid elevation of cytosolic calcium, and the generation of a detectable, concentration-dependent bioluminescent signal. Dimerization of receptors upon ligand binding, and the subsequent increase in cytosolic Ca²⁺ concentration, is characteristic of many 1-TM receptors (Heldin (1995), Cell, 80:213-23), and is one method of detection for the expressed, self-assembling protein array. In other cell-based biosensor examples, cells have been used to produce proteins at specific locations in the array (Ramachandran, et al. (2004), Science, 305:86-90).

One example of a limitation of most-cell based biosensors is that the creation of new sensors requires extensive time and effort. For many cell-based biosensors, each new target of interest (e.g. a new pathogen, antibody, or protein) requires a new cell line to be created, requiring extensive cloning, selection, and testing steps.

Another example of a limitation of most-cell based biosensors is that multiplexed detection in a convenient, miniaturized format is not feasible. The use of more than a handful of sensors simultaneously is impractical. To detect hundreds of samples, one would need to maintain hundreds of individual cell lines. Because the cells are not arranged in any type of position-specific format (such as an array), the signal from one cell cannot be differentiated from another, even if the cells possess different target specificities.

The present invention combines the power of multiplexed protein arrays with the detection sensitivity of cell-based biosensors. The arrays described herein offer the capability of detecting dozens to hundreds of proteins simultaneously by using a stable microarray format that can automatically signal in response to target binding. The arrays describe herein overcome the major limitations of protein arrays (expensive production, poor stability, and low detection sensitivity) while offering the benefits of cell-based biosensors (high sensitivity and label-free detection). The arrays can comprise an array of self-reporting sensors specific for particular targets. Each self-reporting sensor can be a modular protein comprising an extracellular target-specific recognition domain (e.g. Fab or HA1 fragments) and an intracellular 1-TM signaling domain (e.g. the signaling domain of PDGFR). By designing modular cassettes, large libraries of receptors able to recognize diverse pathogens, antibodies, and other targets can be assembled.

The present invention is also related to immunofocusing. The viral coat protein Envelope (Env, or gp160) is a primary immunogenic determinant of HIV-1. However, to avoid recognition by the immune system during infection, native gp160 shields conserved antigenic regions. The existence of broadly neutralizing antibodies in some cases of natural infection suggests that protective immunity can be conferred by vaccination if the appropriate immunogens are used. However, immunogens that elicit broadly neutralizing antibodies (rather than strain-specific antibodies) have been difficult to isolate or design. The mutation array, described herein, is used here to screen mutants of gp160 in which conserved antigenic sites are exposed, and strain-specific epitopes are eliminated.

Immunofocusing is a major objective of vaccine development (Burton, et al. (2004), Nat Immunol, 5:233-6). The strategy is, for example, to 1) expose conserved epitopes, and 2) remove immunodominant decoy epitopes (such as variable loops). For example, using 81 point mutations and 3 loop deletions, monomeric HIV-1 gp120 was engineered to enhance exposure of the b12 binding site (3-10 fold increases in affinity for 6 mutants) and reduce binding of non-neutralizing antibodies (Pantophlet, et al. (2003), J Virol, 77:642-58, Pantophlet, et al. (2004), Protein Eng Des Sel, 17:749-58). Similarly, 21 Ala-scan mutations around and including the core 4E10 epitope revealed that 8 substitutions could (individually) increase 4E10 neutralization sensitivity by 3-30 fold (Zwick, et al. (2005), J Virol, 79:1252-61). These data demonstrate how the structural context of Env can profoundly influence the exposure and presentation of epitopes such as those important in b12 and 4E10 binding. Complementing these findings, substantial evidence suggests that variable loop deletions can result in better anti-viral protection by eliminating immunodominant but strain-specific structures (Barnett, et al. (2001), J Virol, 75:5526-40, Kolchinsky, et al. (2001), J. Virol., 75:3435-3443, Yang, et al. (2004), J Virol, 78:4029-36). Combined, an immunofocused Env protein may be capable of generating sufficient levels of broadly neutralizing antibodies to constitute an effective vaccine immunogen. In some embodiments, the present invention is used to derive an improved immunogen. The improved immunogen can be used for the development of vaccines and drugs. In some embodiments, additional applications include the identification of amino acids that contribute to drug resistance, and analysis of proteins that contribute to viral maturation.

The present invention relates to collections (libraries) of gene(s) arranged in an array or other format that facilitates cell expression for structural and/or functional analyses of gene products. In some embodiments of the present invention the collection is in the form of a mutation array, comprising mutated variants of at least one gene, the DNA of which are arranged in a format appropriate for large-scale transfection of cells. In some embodiments, the format is pre-determined such that the contents of each location are known (e.g. the mutation is known). In some embodiments, the contents of each location are not known, but can be determined by routine experimentation (e.g. sequencing, RT-PCR, cloning, transformation, and the like).

The nucleotide library used to create a cell array can be composed of two different types: 1) a library in which each spot represents a different gene (e.g. an entire genome or clones from a cDNA library), or 2) a library in which each spot represents a different mutation of the same gene (e.g. a 10,000 spot array of a 1,500 bp gene could represent every possible single amino acid mutation). In some embodiments, the library containing locations or spots that represent a different gene comprise about 1 to about 100 genes, 1 to about 200, 1 to about 500, 1 to about 1000, 1 to about 10,000, about 1000, about 2000, about 3000, about 4000, about 5,000, about 6,000, about 7,000, about 10,000, at least 2, at least 5, at least 10, at least 20, at least 30, at least 40, at least 50, at least 100, at least 200, at least 500, at least 750, at least 1,000, at least 2,000, at least 3,000, at least 10,000, about 100 to about 1000, about 200 to about 1000, about 500 to about 1000, about 1000 to about 2000, about 2000 to about 10000 different genes.

In some embodiments, the present invention provides a library of mutated variants of at least one gene arranged in array format, and expressed in mammalian or other cells by a process known as “reverse transfection.” In some embodiments, the array comprises nucleic acid molecules comprising polymorphisms of one gene. A “polymorphism” is a change in a gene that is either naturally produced or is introduced into the gene by a specific mutation made by one of skilled in the art. By “naturally produced” it is meant that the polymorphism is found in nature.

Examples of the invention described herein include, but are not limited to, random mutagenesis of the 352 amino acid CCR5 protein and analysis of a 2,000 clone library of this CCR5 random mutagenesis (representing changes at each position throughout the protein). Another example includes an Ala-scan mutagenesis of the 523 amino acid Kv1.3 ion channel, resulting in 522 clones, each with a single amino acid change to Ala (the Met start codon is excluded from this and most other mutagenesis). Another example includes mutagenesis of the 36 amino acid HR1 region of HIV-1 gp41, changing each individual position in this region to every other possible amino acid, thus resulting in 36×19=684 mutations, each of which is analyzed in the library by reverse-transfection.

In some embodiments, the array comprises a sensor and a reporter. The sensor and/or reporter can be encoded for by the nucleic acid molecules in the array. In some embodiments, the sensor is an antibody or antibody-like molecule. The sensor can be expressed on the surface of the cell. In some embodiments, the sensor is present in the cell prior to the reverse transfection. In some embodiments, the sensor is encoded by DNA at a particular location on the array. As used herein the term “antibody-like molecule” refers to a molecule that is an antibody or a fragment of an antibody. In some embodiments, an antibody-like molecule comprises a monoclonal antibody, a polyclonal antibody, an affinity-purified polyclonal antibody, a Fab fragment derived from a monoclonal antibody, an immunoglobulin-fusion protein, a single chain Fv, an Fc-fusion protein, or combinations thereof. An antibody-like molecule may also encompass non-protein based recognition elements, such as aptamers (Iqbal, et al. (2000), Biosens Bioelectron, 15:549-78, Nimjee, et al. (2005), Annu Rev Med, 56:555-83)

The sensor can be any molecule that can be used to sense the presence of another molecule. Thus, a sensor can be, for example, an antibody that recognizes a specific protein or a epitope-tag (e.g. HA, Myc, etc.) The reporter can also be any reporter molecule that can be used to determine when a sensor binds to its other component. The reporter can be, for example, FRET, BRET, or complementation.

One method of measuring protein-protein interactions is by fluorescent resonance energy transfer (FRET). When complementary fluorescent reporters are brought into close proximity, the transfer of fluorescent energy from an excited donor (CFP) to an acceptor (YFP) results in fluorescence emission by the acceptor (Stanley (2003), Chroma Application Note No. 6). The transfer of energy is by non-radiative dipole-dipole interaction, making FRET efficiency highly dependent upon fluorochrome pair proximity (within 5 nm), and thus an excellent indicator of proximity. FRET strategies have been used on numerous occasions within cells and cell-based biosensors to measure interactions between membrane proteins (Chan, et al. (2001), Cytometry, 44:361-8, Minor (2003), Curr Opin Drug Discov Devel, 6:760-5, Overton, et al. (2000), Curr Biol, 10:341-4, Overton, et al. (2002), Methods, 27:324-32, Tertoolen, et al. (2001), BMC Cell Biol, 2:8). A similar system using a luminescent and fluorescent pair is also available (BRET) (Perkin Elmer).

Any cell that can be transfected can be used in reverse transfection. Cells that can be reverse transfected include, but are not limited to, HEK-293 cells, QT6 cells, 3T3 cells, HeLa cells, BHK cells, CHO cells, Cos cells, CCC cells, CF2TH cells, derivatives of these cells (e.g. stable transfectants, sub-clones), and the like.

In some embodiments, the nucleic acid molecule is deposited on the surface of the array at a concentration of 0.01 μg/μl to about 10 μg/μl, 0.01 μg/μl to about 5 μg/μl, 0.01 μg/μl to about 3 μg/μl, 0.01 to about 2.0 μg/μl, 0.01 to about 1 μg/μl, greater than 5 μg/μl, greater than 6 μg/μl, greater than 7 μg/μl, 0.01 to about 0.5 μg/μl.

As used herein, the term “array format” can refer to an array on any surface or container, including but not limited to a slide, a multi-well plate, a microplate, plastic, glass, nitrocellulose, and the like.

As used herein, the term “array” refers to one or more compositions placed in an array format in or on a particular surface or in a container. For example, the composition can be placed onto a flat surface that has no wells. Alternatively, the composition can be placed within a well, where the wells are arranged in an array format. Examples of surfaces or containers include, but are not limited to, chips, slides, microplates, multi-well plates, microplate well, and the like. In some embodiments, the array is a microarray. A “multi-well plate” can be a plate with actual wells built in or a slide that is used for spotting compositions onto the slide as if there were wells. An “array within a microplate well” is, for example, when a well in a 96-well plate is spotted with more than one spot. This can also be referred to as an “array-within-array.” The array can be any size, for example, standard plate sizes are 8×12, 16×24, 32×48. For array-within-array, standard sizes of the array (within each well) are, for example, 2×2, 3×3, 4×,4, 5×5, 6×6, 8×8, 10×10. Arrays can also be based on pin configurations, with a 2-4 rows of 4-12 pins each typical. Each touch of the pinhead can print multiple spots. In some embodiments arrays are often 100-300 spots per slide. In some embodiments, arrays can be 2,000 spots per slide. High density arrays can be, for example, 10,000 spots per slide or even higher. In some embodiments, the array will comprises 100 to 5,000 spots.

As used herein, the term “about” refers to a range of ±10% of the number that is being modified. For example, the phrase “about 10” would include both 9.0 and 11.0.

The articles “a”, “an”, and “the” are used herein to refer to one or to more than one (i.e., to at least one) of the grammatical object of the article. By way of example, “an element” means one element or more than one element.

The library of mutant variants can comprise any or all amino acid changes of at least one gene. In some embodiments, the gene is mammalian or non-mammalian or is from a eukaryotic or non-eukaryotic cell. Examples of mammalian genes include, but are not limited to, mouse, human, dog, cat, rat, pig, monkey, chimpanzee, ape, gorilla, and the like. Examples of non-mammalian genes include, but are not limited, to, bacteria (e.g. E. coli), yeast, drosophila, worms (C. elegans), and the like.

As used herein, the term “reverse transfection” refers to a process where cells are transfected with a nucleic acid compound by introduction of cells to a surface upon which DNA, desiccated or frozen onto, and other necessary supplements are present. In some embodiments, the present invention can be used for the systematic structural and functional analyses of proteins, including, but not limited to, intracellular, membrane, and secreted proteins and their respective mutants. Nucleic acid compounds include, but are not limited to DNA, RNA, plasmids, viral vectors, viruses, oligonucleotide compounds (e.g. antisense compounds and RNAi compounds), and the like. The nucleic acid compounds can be prepared by any method including synthetically or recombinantly by isolating the nucleic acid compound from a cellular source. In some embodiments, the nucleic acid molecules are prepared and/or isolated using kits. Any kit can be used. Examples of kits can be found in the product catalogs of Qiagen, Invitrogen, Promega, Eppendorff, Biorad, and the like. The nucleic acid molecules can be prepared and/or purified with a maxiprep kit, a mini-prep kit, cesium chloride prep, and the like.

A nucleic acid molecule can also encode part of a virus. A “part of a virus” is a protein or other nucleic acid product is the present in a virus. In some embodiments, the “part of a virus” is a structural protein that is necessary and/or sufficient for viral particle formation and/or budding.

In some embodiments of the present invention, the cDNAs reverse-transfected into cells encode for an epitope tag that is attached to the translated protein. In some embodiments, this tag facilitates the binding of the translated protein to the surface where the cDNA has been reverse-transfected. In some embodiments, this tag facilitates the binding of the translated protein to a different surface. The tag may be, but is not limited to, V5, HA, myc, AU1, FLAG, His, biotin, or GST.

An array of nucleic acid molecules can refer to an array of DNA, RNA, plasmids, viral vectors, viruses, oligonucleotide compounds (e.g. antisense compounds and RNAi compounds, e.g. siRNA), and the like.

As used herein, the term “oligonucleotide compound” refers to a compound comprised of nucleic acid bases that is about 5 to about 400, about 5 to about 300, about 5 to about 200, about 5 to about 100, about 5 to about 80, about 8 to about 80, about 8 to about 50, about 8 to about 50, about 8 to about 40, about 8 to about 30, about 10 to about 30, about 15 to about 30, about 20 to about 30, about 21, about 22, about 23, about 24, about 25, about 26, about 27, about 28, about 29, or about 30 nucleotide bases in length. The oligonucleotide compound can comprise modified bases and/or modified linkages. The oligonucleotide compound can comprise both deoxyribonucleotide bases and ribonucleotide bases. In some embodiments, the oligonucleotide compound is DNA or RNA and can be single stranded or double stranded.

In some embodiments of the present invention, cDNA for a particular gene is inserted into a plasmid vector using standard molecular cloning techniques. Epitope tags flanking the cDNA insert can also be included as desired in order to facilitate detection and characterization of the protein product expressed in cells. Unique restriction sites can be introduced at one or more sites to facilitate removal and isolation from the vector, promoter, and epitope tags, of the cDNA insert in its entirety, or in two or more separate segments. Examples of epitope tags include, but are not limited to, HA, V5, myc, FLAG, AU1, C9, His, and the like. Examples of restriction sites can be found, for example, in the New England BioLabs catalog.

The nucleic acid molecule can encode for many proteins including, but not limited to, diverse cytokines, growth hormones, and steroids, which have been implicated in the control of cell growth and proliferation, and in the pathogenesis of cancers. Mutated or polymorphic forms of these molecules, and their cytoplasmic, nuclear, and membrane receptors have been associated with malignant transformation in a large number of tissues, including stomach, prostate, colon, and breast. Polymorphic forms or splice variants of receptors that are preferentially expressed on cancer cells may therefore be valuable molecular targets for discriminatory anti-cancer therapies. Thus, the nucleic acid arrays can comprise oligonucleotides comprising polymorphisms of a particular nucleic acid sequence or sequences.

In some embodiments of the present invention, one or more desired segments of the cDNA insert are removed and isolated from a parental vector. Nucleotide changes can be introduced into the insert by a procedure such as PCR random mutagenesis. The mutagenesis frequency can be set to introduce a range of nucleotide (and subsequent amino acid) changes per 1000 bp (˜333 aa), as desired. For example, a mutation frequency of 2 bp per 1000 bp is expected to result in approximately 1 amino acid change in the protein product, while a frequency of 4 bp per 1000 bp is expected to result in approximately 2 amino acid changes in the protein product. One of ordinary skill in the art would recognize that a range of technical or academic factors may influence the length of insert segment to be mutated, and the frequency of mutagenesis. Any number of mutations can be inserted into the mutated nucleotide sequence. Following mutagenesis, the cDNA insert can be ligated back into the parental vector, thus ensuring that mutations are introduced only into the desired segment, and not into other segments, the epitope tags, transcriptional elements (e.g. the promoter), or other regions of the plasmid vector. This process may be repeated on different representatives of the same insert or insert segment as many times as desired to develop a series of mutant variants of the same gene.

Any mutagenesis techniques can be used to introduce the mutation in the nucleic acid molecule. Examples can be found, for example, in Molecular Cloning: A Laboratory Manual (3-Volume Set) (Sambrook et al., Cold Spring Harbor Laboratory; 3rd edition (Jan. 15, 2001))(Adereth, et al. (2005), Biotechniques, 38:864, 866, 868, Ali, et al. (1995), Biotechniques, 18:746-50, Bubeck, et al. (2004), J Virol, 78:8026-35, Chatellier, et al. (1995), Anal Biochem, 229:282-90, Delagrave, et al. (2003), Assay Drug Dev Technol, 1:187-98, Lefevre, et al. (1997), Nucleic Acids Res, 25:447-8, Locher, et al. (2005), DNA Cell Biol, 24:256-63, Neuner, et al. (1998), Nucleic Acids Res, 26:1223-7, Vasl, et al. (2004), BioTechniques, 37:726-730, Weiss, et al. (2000), Proc Natl Acad Sci USA, 97:8950-4). Other methods are described in U.S. Pat. No. 6,924,112 Mutagenesis strategies include random mutagenesis, Ala-scan mutagenesis, site-specific mutagenesis, and chimeric recombination. Mutagenesis kits and services can also be used and be purchased from companies including, but not limited to Clontech, Stratagene, Promega, New England BioLabs, Invitrogen, Biomethodes, SeqWright, Blue Heron, and the like.

Any region of a protein can be the focus of mutagenesis. For example, the gene coding for the entire protein can be subjected to mutagenesis. Alternatively, only a small portion of the protein can be the focus of mutagenesis, even a single amino acid (codon). These portions of the protein may be regions of interest within the protein, such as extracellular loops, intracellular loops, or cytoplasmic domains. They can also include specific residues or residues throughout the protein that are of interest, such as charged residues or hydrophobic residues.

As used herein, the abbreviation “Δgene” refers to any mutated variant of a particular gene, and may include the range of unspecified mutations that may result from the mutagenesis procedure(s) described above. Δgenes can be characterized by direct sequencing.

Bacteria, such as E. coli, may be transformed with the Δgene(s) of interest, and cloned by standard culture. A collection of these bacterial clones or their isolated Δgene mutant plasmids, are referred to herein as a “mutant library”.

As used herein a “mutant library” represents at least one change at one or more (or all) amino acid positions, and may be assembled by including and excluding individual Δgenes as desired. For example, mutants in which a premature stop codon is introduced, or in which a conserved nucleotide substitution has been made, may be excluded to produce a library consisting only of Δgenes for which the product is predicted to be full-length and to contain at least one amino acid substitution. Δgene plasmids may be isolated from bacteria using any of a variety of well-known DNA preparation procedures. The mutations may also take place in a region of the nucleotide sequence that does not affect the coding region of the nucleotide sequence but changes the non-coding region. Examples of non-coding regions include, but are not limited to, 5′-UTR, 3′-UTR, transcriptional elements, translational elements, polyA-tail, RNA stabilization elements, introns, and the like.

As used herein, the term “transcriptional elements” refer to elements in a nucleotide sequence that effect transcription. These elements include, but are not limited to, promoters, enhancers, repressors, and the like.

As used herein, the term “translational elements” refer to elements in a nucleotide sequence that effect translation of an mRNA into a protein.

According to an embodiment of the present invention, isolated Δgene plasmids can be mixed with a transfection agent, such as a lipid based transfection agent (e.g. Lipofectamine 2000), and placed onto a surface. The addition of additives, such as sugars (e.g. sucrose or trehalose), may be desirable for increasing stability. A wide variety of substrates, and placement formats may be selected. In some embodiments, each Δgene will be coated on the bottom surface of a separate well in a 96- or 384-well microplate. In another embodiment, Δgenes may be arrayed as spots on an undivided surface, such as a 1×3 inch glass slide. A wide variety of substrate materials, including polystyrene and glass, may be used, and a surface coating, consisting of such materials as polylysine or collagen, may be employed. Placement of the DNA mixture on the substrate may be achieved using an automatic, robotic liquid handling system.

The terms “mutation array” and “mutation expression array”, as used herein, refer to these specified arrangements of Δgenes on any surface and in any format.

In some embodiments, the composition comprising the nucleotide sequence (e.g. the DNA and transfection agent) of a mutation array is allowed to become desiccated and therefore adhere onto the array surface in a form that facilitates short- or long-term storage. In some embodiments, the composition comprising the nucleic acid molecule and the transfection reagent of a mutation array can be frozen in a non-desiccated format to facilitate storage.

As used herein, the term “desiccated” can also be referred to as drying.

In some embodiments, the present invention also provides for frozen multiwell containers comprising an array of cells, a nucleic acid molecule and a transfection reagent. As used herein, the term “container” refers to any object that is capable of containing a cell. Examples of containers include, but are not limited to multiwell plates, including, for example wells and slides, and the like. The number of wells can be any number including, but not limited to 96, 384, 1536, and the like.

In some embodiments of the present invention, cells can be reverse transfected with Δgene DNA by layering the cells onto the array substrate, and allowing them to adhere and to begin to proliferate. Up-take of Δgene DNA by cells will effect expression of the Δgene product, the Δprotein. One of ordinary skill would recognize that a variety of cells and cell culture conditions may be selected to meet specific technical or academic requirements. Selection of cell-type may be made, for example, on the basis of endogenous gene production, transfection efficiency, and expression efficiency. Culture conditions, including culture medium and additives, and culture time, will vary depending upon cell type used, the specific requirements of the expressed protein, and upon the experimental purposes for which expression is being effected. Expression of each Δprotein can be independently characterized by immuno-analysis of the co-expressed epitope tags (e.g. direct immunostaining, flow cytometry, Western blot). Where expression of all or part of the protein or epitope tags is intracellular (such as is the case with cytoplasmic or nuclear proteins, or the cytoplasmic tails of integral membrane proteins), cells may be permeabilized prior to immunoanalysis.

In some embodiments, the present invention provides cells expressing Δproteins from the mutation expression array to be used in structural and functional analyses of the mutated protein. In some embodiments, the cells are recovered as viable cells after being reverse transfected. As used herein, the term “receptor” refers to any protein or Δprotein, integrated into a cellular membrane, solubilized within the cytosol, or secreted in soluble form into the cell environment that interacts with a molecular target.

A “ligand” refers to any molecular species, be it a single protein or other molecule, or a multi-molecular structure containing such a molecular species, that engages in a physical interaction with a protein or receptor. Binding of physiological ligands (e.g. proteins, steroids, chemokines), pathological ligands (e.g. bacteria, viruses, or their proteins), or exogenous ligands (e.g. antibodies, pharmaceutical agents) to proteins or receptors results from the formation of a variety of chemical bonds between a series of individual amino acids of a receptor protein, and those of the ligand. The amino acids that constitute a receptor binding site can be sequential in the primary protein sequence, and hence linear, or may be distant in the primary sequence, but brought into proximity through local or global receptor folding events, and thus be considered conformation-dependent. Differences in ligand-interaction characteristics between wild-type receptors and Δprotein receptors may result: 1) from substitution of amino acids that directly interact with the ligand (thus causing decreased or increased attraction between target amino acids); 2) from substitutions of amino acids that cause or reduce steric hindrances between the receptor binding site and the ligand; or 3) from amino acid substitutions that result in alterations to local or global receptor protein folding and tertiary structures, thus enhancing or disrupting the ligand binding site.

Δprotein-ligand interactions may be characterized by direct binding analyses, such as immunoassays and competitive or saturation binding analyses. Functional assays, such as viral infection assays and cell signaling assays may be utilized to elucidate functional ramifications of structural substitutions. By testing the structural and functional capabilities of Δproteins within a mutation expression array (which may include Δproteins with amino acid substitutions at every amino acid position, or any combination of desired target sites within the sequence), a detailed map of amino acid contributions to structural and functional characteristics of the wild-type protein can be constructed.

The present invention also provides for multi-well plates comprising an array of nucleic acid molecules wherein the nucleic acid molecules are attached to the plate by freezing. In some embodiments, the multi-well plate is a 96-, a 384—, a 1536-, or a 6144 well plate. In some embodiments, the multi-well plate is referred to as a microplate.

As used herein, the term “array of nucleic acid molecules” refers to an ordered grouping of nucleic acid molecules. In some embodiments, the array of nucleic acid molecules comprises cDNAs that encode for the same protein product or transcriptional element, but differ by the number or placement of mutations. In some embodiments, the array of nucleic acid molecules comprises cDNAs that encode for different protein products. When the array comprises cDNAs that encode for different products this can also be referred to as a cDNA library. The array of nucleic acid molecules can also comprise nucleic acid molecules that comprise transcriptional elements. In some embodiments, the array comprises the same transcriptional element, but with different mutations or a different number of mutations. In some embodiments, the array comprises different transcriptional elements. For example, an array would be considered to have different cDNAs that encode for different proteins, if one well expresses CCR5 and another well expressed CCR3. An array would be considered to be a “mutation array” when each well or location encodes for the same product, but differs by the mutation in each well or location. In some embodiments, the mutation is an insertion, deletion, substitution, or combinations thereof. In some embodiments, the mutation of a protein product or nucleic acid product is mutated by Alanine scanning mutagenesis, where each well has a different amino acid residue mutated to alanine, or if the residue is an alanine to a glycine residue. An array of nucleic acid molecules can be placed on or in a multi-well plate or on a glass slide.

In some embodiments, instead of an array of nucleic acid molecules, an array of phage are used. The phage can be produced in clones. In some embodiments, the clones can be spotted on a glass slide to form a microarray of phage, allowed to dry, and stored frozen until ready to use. In some embodiments, each clone is placed within a well of a microplate and frozen. When ready for use, a lawn of bacteria is grown on the microarray slide or placed into the microplate well. In some embodiments, the bacteria is TG1. The growing bacteria are infected by location-specific phage and produce the protein coded by that phage. When fixed to the surface, the cells expressing the protein encoded for by the phage are adhered to the slide. When the proteins encoded by each phage contain epitope tags and the surface on which the proteins are produced contain an element that binds the epitope tag, the proteins produced at each location will be adhered to that location and other molecules (phage proteins, cellular proteins) can be washed away, leaving behind the protein of interest.

The present invention also provides for methods of preparing multi-well plates comprising an array of nucleic acid molecules. In some embodiments, the method comprises depositing a composition comprising a nucleic acid molecule into a well of a multi-well plate; and/or freezing the multi-well plate. In some embodiments, the nucleic acid molecule is deposited onto a surface at 4° C. In some embodiments, the nucleic acid molecule is spotted on to the surface (e.g. multi-well plate or slide).

In some embodiments, the composition comprises additives that can stabilize the nucleic acid molecules for long-term storage or help enhance the transfection efficiency. In some embodiments, the composition comprising the nucleic molecules comprises transfection reagents, sugars, and a gelatin component. In some embodiments, the sugar is sucrose or trehalose. In some embodiments the gelatin component is a protein gelatin, a hydrogel, a sugar-based gelatin or a synthetic gelatin. However, in some embodiments gelatin is not necessary for the reverse transfection to work. Accordingly, in some embodiments, the composition comprising a nucleic acid molecule does not comprise a gelatin component. In some embodiments, the composition comprising the nucleic molecules comprises transfection reagents, sugars, and/or an EDTA component. In some embodiments, the composition is stored in a multi-well plate with a hyrdrogel. In some embodiments, the composition is stored in a multi-well plate with a hyrdrogel provided that the composition is not allowed to dry on the plate.

In some embodiments, the sugar is present in the composition at about 0.01% to about 1%, about 0.1%, about 0.2%, about 0.3%, about 0.4%, about 0.5%, about 1%, about 0.1% to about 1.0%, about 0.1% to about 5.0%, about 0.1% to about 4.0%, about 0.1% to about 3.0%, about 0.1% to about 2.0%, about 0.1% to about 0.5%, or about 0.1 to about 0.25%.

In some embodiments, the EDTA is present in the composition at about 1-50 mM. In some embodiments, the EDTA is present in the composition at about 0.01% to about 1%, about 0.1%, about 0.2%, about 0.3%, about 0.4%, about 0.5%, about 1%, about 0.1% to about 1.0%, about 0.1% to about 5.0%, about 0.1% to about 4.0%, about 0.1% to about 3.0%, about 0.1% to about 2.0%, about 0.1% to about 0.5%, or about 0.1 to about 0.25%.

As used herein, the term “transfection reagent” refers to a composition that is used to facilitate the transfection of a nucleic acid molecule into a cell. In some embodiments, the transfection reagent is a lipid (monocationic or polycationic), non-liposomal, or lipid-like based transfection reagent, calcium phosphate based transfection reagent, DEAE-Dextran, Activated Dendrimers (spherical polyamindoamine molecules with branches radiating from a central core, terminating at charged amino groups), peptide, polymer, and the like. Transfection reagents and methods of transfection are also described in (Ausubel, et al. (2001), Current Protocols in Molecular Biology, Brokx, et al. (2004), Methods Mol Med, 90:139-60), (The Scientist 11[19]:18, Sep. 29, 1997), which also can be used. A transfection reagent can also be a reagent that is used to electroporated a molecule into a cell. In some embodiments, the nucleic acid molecule is electroporated into a cell.

A composition comprising a nucleic acid molecule that can be electroporated into a cell refers to a composition that is suitable for electroporation. One of skill in the art can readily determine the appropriate conditions for a composition to be suitable for electroporation.

Examples of transfection reagents include, but are not limited to, Lipofectamine, Lipofectamine Plus, Lipofectamine 2000, Effectene, Fugene, Lipofectin, Avanti Transfectant reagent, SureFECTOR, UniFECTOR, COSFectin, HEKFectin, TransFectin, Cellfectin, DMRIE-C, Optifect, LyoVec, LipoGen, GeneTrans, DreamFect, Tfx-50, Tfx-10, Tfx-20, TransFast, Transfectam, DOSPER, DOTAP, X-tremeGENE, DOTAP methosulfate, LipoTAXI, polyethylenimine (PEI), transferrin, cellphect, genechoice transfectol, DEAE-Dextran, calcium phosphate, Lipogen, Transit, GeneJuiice, SAINT Transfection reagent, Fecturin, jetPEI, PolyFect, SuperFect, ESCORT transfection reagent, GeneJammer, and the like. Transfection reagents include, but are not limited to, chemical precipitates, liposomal reagents, non-liposomal reagents, and polymers.

In some embodiments, the array of nucleic acids that are attached to the multi-well plate are attached by freezing. The plate can be frozen by any method. In some embodiments, the plate is chilled at about 0° C. (e.g. on ice) before being frozen. In some embodiments, the plate can be frozen from about 0° C. to −170° C. In some embodiments, the plate is frozen at a temperature that is less than 0° C., less than −10° C., less than −20° C., less than −30° C., less than −40° C., less than −50° C., less than −60° C., less than −70° C., less than 75° C., less than −79° C. or at a temperature of −10° C., −20° C., −30° C., −40° C., −50° C., −60° C., −70° C., −80° C. In some embodiments, the plate is frozen at a temperature between 0 and −10° C., −11 and −20° C., −21 and −30° C., −31 and −40° C., −41 and −50° C., −51 and −60° C., −61 and 70° C., 71−80° C., −81−90° C., or −91 to −170° C.

As used herein, the term “attached” when referring to the nucleic acid molecule being attached to a plate or slide refers to a composition comprising a nucleic acid molecule being physically associated with the surface (e.g. slide, multi-well plate, microplate, and the like). It can be attached by either freezing the composition or desiccating the composition such that the dried material is in contact with the surface. “Attached to a surface” can also refer to being “contained in” a surface (e.g., a multi-well plate).

In some embodiments, the nucleic acid molecule is a plasmid, virus, or viral vector. In some embodiments, the nucleic acid molecule is free of infectious agents. As used herein, the term “infectious agents” refers to a virus or viral-based vector.

In some embodiments, the nucleic acid molecule encodes for a protein. In some embodiments, the nucleic acid molecule encodes for a transcriptional element. In some embodiments, the transcriptional element is operably linked to a reporter gene, which allows the activity of the transcriptional element to be measured. In some embodiments, the reporter gene is a fluorescent protein or enzymatic protein. Examples of fluorescent protein include, but are not limited to GFP, YFP, CFP, and the like. Examples of reporter proteins that are enzymatic proteins are alkaline phosphates (e.g. secreted alkaline phosphatase), luciferase, β-galactosidase, and the like.

As discussed above, in some embodiments, the nucleic acid molecule encoding for the protein is mutated. In some embodiments, the locations or well comprising the nucleic acid molecule comprises a different mutation in the nucleic acid molecule. In some embodiments, the nucleic acid molecule comprises 1, 2, 3, 4, 5, 6, 7, 8, 9, 10, 1-3, 2-5, 5-10, at least 2, at least 3, at least 4, at least 5, at least 6, at least 7, at least 8, at least 9, or at least 10 mutations. In some embodiments, the number of mutations refers to the number of base pairs that are mutated. In some embodiments, the number of mutations refers to the number of amino acid residues that are mutated in a peptide or protein that is encoded by the nucleic acid molecule that is mutated. In some embodiments, the mutation is a silent mutation, insertion, substitution, deletion, or a frameshift mutation. In some embodiments, the mutation consists of a chimeric molecule created by fusing together portions of two parental molecules at one or more locations, for example CCR5-CCR2b chimeras as described (Rucker, et al. (1996), Cell, 87:437-446).

In some embodiments, each well or location comprises a nucleic acid molecule comprising a different cDNA. In some embodiments, the nucleic acid molecule is a member of a cDNA library.

The present invention also provides methods of introducing nucleic acid molecules into cells. In some embodiments, the method comprises thawing a multi-well plate comprising a composition comprising a nucleic acid molecule; and contacting a well with the cell under appropriate conditions for entry of the nucleic acid molecule into the cell. In some embodiments, the cells are recovered after the cells have been contacted with the nucleic acid molecule. In some embodiments, the cells that are contacted with the nucleic acid molecule are recovered.

As used herein, the term “cells are recovered” refers to removing the cells from the microplate for further use, e.g., processing them outside of the plate, and/or propagating the cells to produce a cell culture.

In some embodiments, the cells are contacted with the array of nucleic acid molecules under conditions such that the cells are transfected. The reverse-transfection can take place at any temperature that allows the cells to be reverse transfected, e.g. ambient temperature. Examples of temperatures that can be used include, but are not limited to 16° C., 20° C., 25° C., 30° C., 37° C., 30-40° C., 20-30° C., 20-40° C., 15-40° C., 15-37° C., 20-37° C., or 30-37° C.

The present invention also provides for methods of mapping a function of a nucleic acid molecule product. In some embodiments, the method comprises contacting an array of nucleic acid molecules with a cell under appropriate conditions for entry of the nucleic acid molecule into the cell; and measuring a function of the nucleic acid molecule product. In some embodiments, the cells are recovered. In some embodiments, the array of the nucleic acid molecules is deposited on a multi-well plate (e.g. a microplate), a slide, or a microplate.

In some embodiments of the present invention, the multi-well plate comprising the array of nucleic acid molecules is thawed prior to being contacted with the cell.

In some embodiments, the surface that the composition comprising the nucleic acid molecule is attached is coated with a substance that increases cell adhesion. In some embodiments, the surface is coated with collagen prior to attaching the composition comprising the nucleic acid molecule.

As used herein, the term “nucleic acid molecule product” refers to the product encoded by the nucleic acid molecule. The product can be a protein, peptide, antibody, and the like or another nucleotide based compound including, but not limited to, transcriptional elements, dsRNA, antisense oligonucleotide compounds, and the like. In some embodiments, the nucleic acid product encodes proteins derived from pathogenic organisms (e.g. bacteria and/or viruses).

In some embodiments, a composition comprising a nucleic acid molecule without a transfection reagent is dried on the surface. Then, in some embodiments, prior to contacting the cells with the nucleic acid molecule a transfection reagent is added. In some embodiments, the transfection is contacted with the cells at least 15 minutes, at least 30 minutes, at least 1 hour, about 15 minutes, about 30 minutes, or about 1 hour before the cells are contacted with the nucleic acid molecule. In some embodiments, the cells and the transfection reagent are contacted with the nucleic acid molecule at the same time.

The present invention can be used to identify residues that are involved in the function of a protein. In some embodiments, the protein is a kinase, receptor, transmembrane protein, membrane protein, viral protein, a cell surface protein, cytoplasmic protein, secreted protein, ion channel, GPCR, transporter protein, bacterial protein, pathogenic protein, or enzyme. By making an array of nucleic acid molecules that encode for different mutations of the same protein, one can identify the residues involved in that function. The function can be any function including, but not limited to an enzymatic function, binding function, cellular function, signaling function, expression function, and the like.

The function can be measured by any method including, but not limited to, different colors, which would allow multiple measurements of varying functions that can be assayed simultaneously using complementary fluorescent measurements. In some embodiments, complementary fluorescent colors are used for immunofluorescence.

As used herein, the term “enzymatic function” refers to the enzymatic properties of the protein encoded for by the array of nucleic acid molecules.

As used herein, the term “binding function” refers to the ability of the nucleic acid molecule product (e.g. a protein, peptide or transcription element) to bind to a binding partner. In some embodiments, the binding partner is an antibody, a ligand, a small molecule, another protein, a peptide, and the like. In some embodiments, the binding partner of the transcriptional element is a transcription factor. A “transcription factor” refers to a compound that facilitates or inhibits transcription. A transcription factor can be another nucleic acid molecule, organic molecule, a protein, or a peptide.

As used herein, the term “cellular function” refers to a cellular property that can be measured in the cell that has been contacted with the nucleic acid molecule. Any cellular function that can be measured is applicable. Examples of cellular functions include ion concentration, cell death, cell viability, cell cycle stage, membrane potential, changes in protein or RNA expression of genes that are not encoded by the nucleic acid molecule. Other examples include, but are not limited to, generation of secondary messengers, GPCR function, phosphorylation status of a cellular protein, the cell releasing a cellular product into the cell culture medium, a phenotypic change in the appearance of the cell, the location or trafficking of a protein within the cell, and the like.

As used herein, the term “expression function” refers to the expression of the nucleic acid molecule that is contacted with the cell. In some embodiments, the expression of the protein encoded by the nucleic acid molecule is measured. In some embodiments, the RNA and/or mRNA expression of the protein encoded by the nucleic acid molecule is measured.

In some embodiments, the stability of the mRNA that is expressed by the nucleic acid molecule is measured.

In some embodiments, the array of nucleic acid molecules comprises multiple mutations of the nucleic acid product and the function of each product is compared to the wild-type product. Comparing the mutants to the wild-type product can provide information as to which nucleotides or amino acid residues are involved in the function that is being measured.

The present invention also provides methods of preparing an array of viable cells in a multi-well plate comprising thawing a frozen multi-well plate comprising an array of nucleic acid molecules; and contacting a well in the multi-well plate with the cells. In some embodiments, the cells are transfected with the nucleic acid molecule. In some embodiments, the transfected cells are recovered.

As discussed above the array can contain different mutations of a protein in different locations. However, each location in the array does not have to have a different mutation. In some embodiments, some locations may have no nucleic acid molecule or may have a mutation that is already represented in the array. By having multiple locations in the array with the identical nucleic acid molecule one can perform the same experiment at the same time under the same conditions. Locations in either a multi-well plate, microplate, or slide that do not have a nucleic acid molecule can serve as a negative control.

As described above, the present invention can be used to map the function of a protein. An example of a function that can be mapped is ligand-protein interaction. An array of nucleic acid molecules can be prepared that encode for the wild-type and mutations of the protein. The nucleic acid molecules then can be reverse transfected into cells. Once the cells express the native protein and the mutated forms, one can measure the interaction between the protein and its ligands. A weaker binding of the ligand to the mutated protein as compared to the native (wild-type) protein would be indicative that the mutation(s) present in the mutated protein are important for ligand binding. The mutation array comprising the different mutations in the protein can be used to identify all or most of the residues that are critical for the protein to bind to its ligand or binding partner. Measuring the binding between the ligand and the protein can be done by any method including, but not limited to, changes in fluorescence or activity of the protein. Examples of ligands include, but are not limited to an antibody, protein, peptide, small molecule, chemokine, and the like. Thus the present invention can be used to map the epitope of where an antibody binds to another protein. Other physical interactions can also be mapped to determine key interaction residues for the binding of a drug to a protein.

Residues can also be identified that may be involved in drug resistance. A drug that is known to interact with a specific protein or nucleic acid molecule product can be tested against the native and the mutated forms in the cell. The cell will express the protein or nucleic acid molecule product (e.g. transcriptional element) and the interaction with the known drug can be measured. If the drug can no longer interact and the protein or nucleic acid molecule product is still functional, then this would indicate that the mutated base or residue is important in drug resistance for that drug. Therefore, drug resistant mutants of proteins can be identified.

The present invention also provides for facile methods of identifying temperature sensitive mutants of a protein. A mutation array can be prepared that is then reverse transfected into cells. The function of the proteins can then be measured at different temperatures to determine if the mutation is temperature-sensitive. A “temperature-sensitive” mutation refers to a mutation in the protein that is permissive at one temperature and restrictive at a different temperature. In some embodiments, the permissive temperature is less than the restrictive temperature. In some embodiments, the permissive temperature is higher than the restrictive temperature. In some embodiments, the temperature-sensitive mutation changes the structure of the protein.

In addition to using the present invention for functional and structural screening of large libraries of mutated variants of a single gene, the present invention provides for methods of screening other types of libraries, such as tissue-specific mammalian cDNA expression libraries. The present invention can identify receptors for novel ligands or for viral entry. For example, if a ligand is identified, but a binding partner is not known, an array of nucleic acid molecules can be used to express a cDNA library by reverse transfection. The ligand can then be contacted with the reverse transfected cells to identify a binding partner.

An array of nucleic acid molecules that encodes a cDNA library that is reverse transfected can also be used to identify receptors or co-receptors for viral entry. Cells that have been reverse transfected with an array of nucleic acid molecules can be contacted with a virus and entry can be measured. Cells that allow entry into a previously non-permissive cell must express a protein that is necessary for viral entry. A further example is described below.

The present invention also provides methods of selecting a vaccine candidate. Many immunogens that are used as potential vaccine candidates fail because of bindings with antibodies that are not sufficient for protection or because variable regions of the protein (i.e. that differ to widely from strain to strain of a virus) preferentially bind antibodies, making identifying a good vaccine candidate difficult. Thus the present method can identify immunogens that have the good feature of being recognized by an antibody that can generate a therapeutic response (neutralizing antibody), while not being recognized by non-protective antibodies. In some embodiments, the method comprises contacting a composition comprising an array of nucleic acid molecules which encodes variants of a potential candidate (i.e. viral protein or other pathogenic protein) with a cell under appropriate conditions for entry of the nucleic acid molecule into the cell and measuring the binding of the potential candidate to a first antibody and to a second antibody. In some embodiments, the first antibody is a neutralizing antibody and the second antibody is a non-protective binding antibody. If the first antibody binds to the candidate and the second antibody does not, this indicates that the potential candidate can be used. The selected candidate can be improved through an iterative process where mutations that cause the potential candidates to be a selected candidate are combined, swapped, or have other mutations introduced to them randomly or selectively. The same methods are used to test binding to see if binding can be improved, and therefore, are potentially generate more protective immunogens. In some embodiments, the second non-specific antibody is an antibody that binds to a variable region of a pathogenic protein.

The present invention can also be used to identify a drug-resistant mutant of a virus. In some embodiments, the method comprises contacting a composition comprising an array of nucleic acid molecules which encodes the virus with a cell under appropriate conditions for entry of the nucleic acid molecule into the cell and production of the virus. The cell can then be contacted with an anti-viral drug and the growth of the virus is either determined by any means and compared to a virus that is inhibited by the anti-viral drug. If the viral growth is increased when compared to the inhibited virus, the mutant is said to be a drug-resistant mutant. The anti-viral drug can be any composition that is used to inhibit the growth of a virus.

The present invention also provides for methods of producing a virus. In some embodiments, the method comprises contacting a composition comprising an array of nucleic acid molecules which encodes one or more genes from a virus with a cell under appropriate conditions for entry of the nucleic acid molecule into the cell. In some embodiments, the method comprise contacting the cell with one or more nucleic acid molecules that encode one or more genes sufficient to produce the rest of the virus. The growth or infectivity of the virus can be measured of the virus that is produced by the method described. Any virus can be produced in such a manner including pathogenic, non-pathogenic, mutated viruses, and the like. Examples of viruses that can be produced are HIV, SIV, HPV, HSV, HCV, and the like.

The present invention also provides methods for detecting a pathogen in a sample comprising contacting the sample with an array of cells comprising ligands for the pathogens, wherein the ligands comprise a signaling mechanism that is activated upon binding of said pathogens to the ligands. The signal is then detected, whereby detection of the signal indicates the presence of the pathogen. In some embodiments, the signaling mechanism comprises aequorin.

The present invention also provides methods for detecting a protein in a sample comprising contacting the sample with an array of cells comprising binding partners for the protein, wherein the binding partners comprise a signaling mechanism that is activated upon binding of the protein to the binding partners. When a signal is detected it indicates the presence of the protein. The protein can be any protein that can bind to a binding partner. The protein can be, for example, an antibody.

### EXAMPLES

The invention is now described with reference to the following examples. These examples are provided for the purpose of illustration only and the invention should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results. The appended claims are intended to be construed to include all such embodiments and equivalent variations.

### Example 1

### Random mutagenesis of CCR5

pCAGGS-CCR5 was constructed to contain the full length cDNA for CCR5. The CCR5 insert was constructed to be flanked by two epitope tags that are excluded from the mutagenesis process (using unique restriction sites), but which are expressed with each mutated cDNA: an N-terminal HA tag to normalize cell surface expression, and a C-terminal V5 tag to confirm full-length translation. A unique restriction site was placed in the center of the CCR5 gene (by silent mutation) to allow CCR5 to be mutated approximately 500 bp at a time. By restricting mutation regions to 500 bp, mutations can be identified using a single sequencing reaction. Cellular expression of this construct (CCR5 and both tags) was confirmed by immunofluorescence.

Clontech's Diversify™ PCR Random Mutagenesis kit was selected. The Diversify™ mutation rate can be adjusted from two to eight mutations per 1,000 bp simply by controlling manganese and dGTP concentrations. CCR5 was mutated at a frequency of 2.5 bp per 500 bp (or approximately one to two amino acid changes per clone), a rate that allows sufficient analysis of functional contributions of molecular elements with minimal disruption of protein assembly or global structure (Guo, et al. (2004), Proc Natl Acad Sci USA, 101:9205-10). Each mutated segment was purified from agarose gels, ligated back into the unmodified parental vector using the unique restriction sites flanking CCR5 and in the center of CCR5, and transformed into bacterial cells. Thus, mutations were introduced only in the area of interest and not into the host plasmid, epitope tags, or promoter region. Select clones chosen for reverse transfection were sequenced to identify the mutations introduced.

Alternative strategies for introducing mutations into DNA have been described, including site-directed mutagenesis, enzymatically-induced random mutagenesis, staggered extension process (StEP), chimeric recombination, and shuffling of gene fragments (Ausubel, et al. (2001), Current Protocols in Molecular Biology, Leung, et al. (1989), Technique, 1:11-15, Rucker, et al. (1996), Cell, 87:437-446, Zhao, et al. (1998), Nat Biotechnol, 16:258-61). Any of these techniques can potentially be used to create libraries of mutated cDNAs. One skilled in the art would recognize that any gene could be mutated in a similar manner. Libraries can be re-arrayed after sequencing or after initial analysis has determined that certain clones within the library are not useful (e.g. clones contain no insert or premature stop codons).

### Example 2

### Construction of Focused Mutation Arrays

A library of HIV Env mutations is created to focus on the V3 region of Env, a region that controls HIV tropism and coreceptor specificity. Additional regions of interest, such as the HR1 helix in gp41, could also be targeted. JRFL Env will be used. Random mutagenesis using error-prone PCR is performed (Leung, et al. (1989), Technique, 1:11-15), focusing on the approximately 100 bp region of V3. By mutating this region at a rate of approximately 1.5 bp change per 100 bp, or 1 amino acid change per 33 amino acids, a library of 1,000 clones will contain over 20 amino acid changes per amino acid position (i.e. approximately all possible mutations at each residue). Using this library, a mutation array is used to map coreceptor binding sites for JRFL Env.

### Example 3

### Codon Mutagenesis

Mutation Arrays containing random variants of a protein can result in the identification of critical amino acid positions involved in drug binding and protein function. However, the precise contribution of amino acid side-chain structures at each position requires further analysis. By mutating specific amino acid positions to every other possible amino acid, the contribution of side-chain structures at specific amino acid positions can be determined. This data can also be used to construct detailed three-dimensional models of interactions and structures.

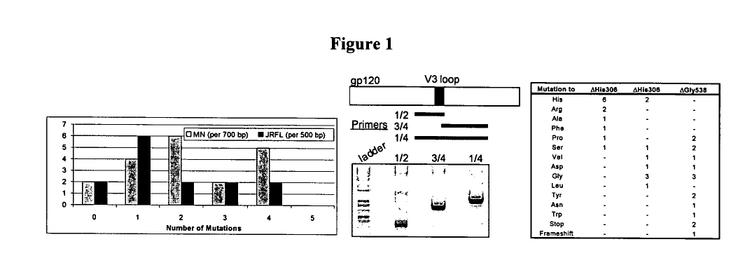

The V3 loop of Env is a major immunogen and determinant of tropism (Hwang, et al. (1991), Science, 253:71-74, O'Brien, et al. (1990), Nature, 348:69-73, Schupbach, et al. (1984), Science, 224:503-505). Similarly, the HR1 helical region of gp41 facilitates membrane fusion and is the target for new fusion-inhibitor drugs (Chen, et al. (1995), J. Virol., 69:3771-3777, Kilby, et al. (1998), Nat. Med., 4:1302-1307, Wild, et al. (1994), Proc. Natl. Acad. Sci. USA, 91:9770-9774). To allow for detailed analysis in areas of interest, an overlapping PCR approach (Ausubel, et al. (2001), Current Protocols in Molecular Biology) was used to introduce random mutations at select codon positions within the V3 and HR1 regions of JRFL Env, including residues previously implicated in the development of drug resistance. The general strategy of mutagenesis is shown in FIG. 1, highlighting the four primers and three PCR products generated. In this strategy, a primer was synthesized with random nucleotides at a particular codon. A larger portion of the gene was then generated using overlapping PCR, and the purified PCR product was cloned into the parental vector using convenient restriction sites near the end of the final PCR product. Two independent rounds of His306 mutation were conducted, and twenty-one clones were sequenced. The resulting codons are indicated for each round in FIG. 1, as well as a separate round of mutagenesis on Gly538 (part of the HR1 helix in gp41). The resulting mutations were localized to the codon of interest and contain codon changes introducing random amino acids. Seven different codons were involved in the mutagenesis of His306 to Arg, Ser, and Gly (2-3 clones of each). The efficiency of this approach achieved 81% (FIG. 1), as measured by the frequency of non-wild type codons. The advantage of this strategy is that even divergent codons will be represented at the location of interest.

### Example 4

### CCR5 Plasmid can be Arrayed, Transfected, and Expressed for High Throughput Analysis

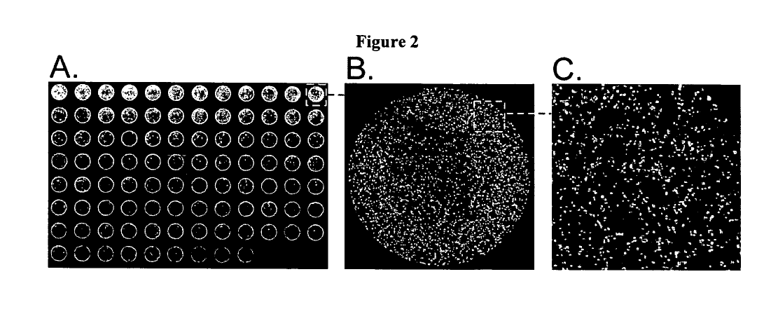

To test whether reverse transfection methodology could achieve expression of CCR5 within living cells in a format suitable for high throughput screening, a 96-well microplate was prepared for the reverse transfection of CCR5. 14 μg of a CCR5 expression vector (pcDNA3-CCR5-GFP) resuspended in 1.75 ml of Optimem was mixed with 35 μl Lipofectamine 2000 in 1.75 ml of Optimem, and allowed to incubate for 20 min. Sucrose was added to 1% final, and 35 μl of the mixture was placed in each well of a 96-well microplate. The mixture was allowed to air-dry overnight. The CCR5 construct used here contained a GFP C-terminal tag to facilitate detection of protein expression. After air-drying of the plasmid-lipid complex to the surface, the plate was stored at −20° C. When ready for use, 5×10⁴ HEK-293T cells were added to each well in 100 μl of 10% DMEM. The cells were allowed to adhere to the well surfaces, become transfected with the plasmid, and express CCR5 at 37° C. for 24 hours. Protein expression within living transfected cells was visualized by imaging for green fluorescence (FIG. 2), indicating the successful expression of CCR5-GFP in 80-90% of the cells per well. Control wells that did not receive plasmid (bottom three wells of FIG. 2A) contained the same number of cells, but were not fluorescent, as expected.

### Example 5

### Frozen Cell Array Preparation

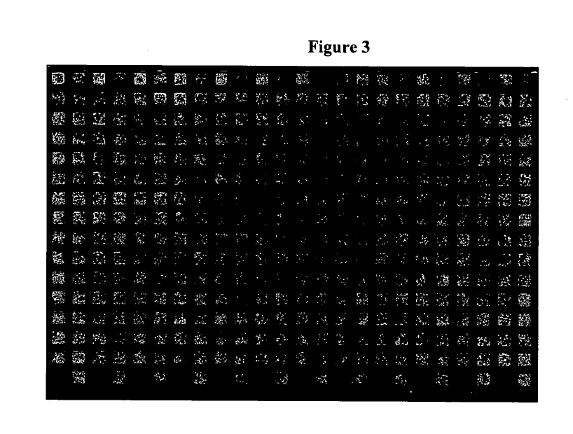

A 384-well microplate was prepared for the reverse transfection of CCR5. 15 μg of a CCR5 expression vector (pcDNA3-CCR5-GFP) was mixed with 40 PI Lipofectamine 2000 and 0.1M sucrose in 2 ml of Optimem, and 10 μl of this mixture was placed into each well. The CCR5 construct used here contained a GFP C-terminal tag to facilitate detection of protein expression. The mixture was frozen at −80C, rather than allowed to dry. When ready for use, the plate was allowed to thaw, and 0.75×10⁴ HEK-293 cells were added to each well in 401 μl of DMEM containing 10% FBS. The cells were allowed to adhere to the well surfaces, become transfected with the plasmid, and express CCR5 at 37° C. for 24 hours. Protein expression within living transfected cells was visualized by imaging for green fluorescence (FIG. 3), indicating the successful expression of CCR5-GFP in 80-90% of the cells per well. Control wells that did not receive plasmid (alternating bottom wells of FIG. 3) contained the same number of cells, but were not fluorescent, as expected. The results demonstrate that CCR5 cDNA can be arrayed, reverse transfected, and expressed without drying of the plasmids in coherent spots.

### Example 6

### Construction of Env Mutation Cell Arrays

MN and JRFL Envs (X4 and R5 strains, respectively) were randomly mutagenized using PCR-based mutagenesis (the Clontech Diversify Mutagenesis kit). Envs were mutated at a desired frequency of 3 bp/kb (FIG. 1). Arrays of MN and JRFL Env mutations were printed with a pin-spotter and tested as described herein in microarray format.

### Example 7

### Cell Arrays can be Constructed in Microarray Format

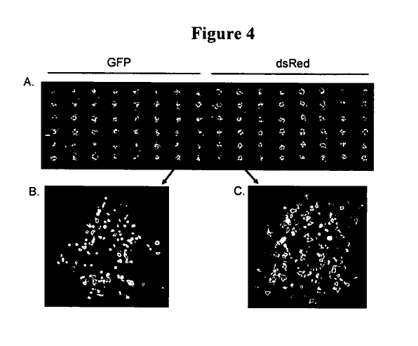

In order to demonstrate reverse transfection in microarray format, a 1×3 inch polylysine-coated glass slide was spotted with plasmids encoding either GFP or dsRED fluorescent proteins. 120 ng of a plasmid expression vector (pcDNA3-GFP or pcDNA3-dsRED) was mixed with 0.3 oil Lipofectamine 2000 and 1% sucrose in 30 ul of Optimem, and spotted onto the surface of each slide with microarrayer pins. Microarray based spotting methods are described in (Schena (2002), 648). After air-drying of the plasmid-lipid complex to the surface, the slide was stored at −20° C. When ready for use, 5×10⁴ HEK-293 cells were added onto the slide in 3 ml of media. The cells were allowed to adhere to the surface, become transfected with the plasmid, and express the fluorescent proteins at 37° C. for 24 hours. Protein expression within living transfected cells was visualized by imaging for green and red fluorescence (FIG. 4), indicating the successful expression of the proteins.

In order to test the specificity of the cell array, a number of plasmids encoding different types of proteins were arrayed in microarray format and detected by reverse-transfection. Proteins included fluorescent intracellular proteins (GFP, dsRED), a single-transmembrane receptor (CD4), HIV-1 Env (MN), and a G-protein coupled receptor (CXCR4). Cell arrays were fixed and stained (without permeabilization) with protein-specific antibodies, including conformation-dependent monoclonal antibodies against CXCR4 and CD4.

In order to determine sources of variability, an experiment was performed using dilutions of plasmids spotted in microarray format for reverse-transfection. The experiment was performed with both dsRED reporter plasmid and with MN Env (detected by staining with anti-HIV human sera). Each experiment contained six dilutions (0 to 125 ng/ul), each dilution set was independently prepared twice, each dilution value was spotted four times, each dilution curve was performed in duplicate on different slides (on the same day), and the entire experiment was repeated twice (two different days). In total, 32 data points were generated per dilution value for each protein, slides 1 & 3 (and 2 & 4) were transfected on the same day, and slides 1 & 2 (and 3 & 4) were printed on the same day with the same plasmid mixtures. Two array pins were used to array all spots. The results demonstrated a general trend of increased fluorescence with increasing DNA concentration. Results indicated that plasmids could be diluted to 25 ng/ul (approximately 1.2 ng in a 50 nl spot) while maintaining detectable fluorescence, both for a fluorescent protein (dsRED) and for an immunostained protein (MN Env).

### Example 8

### Reverse Transfection Optimization

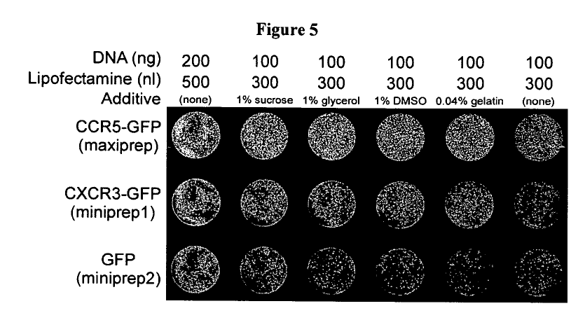

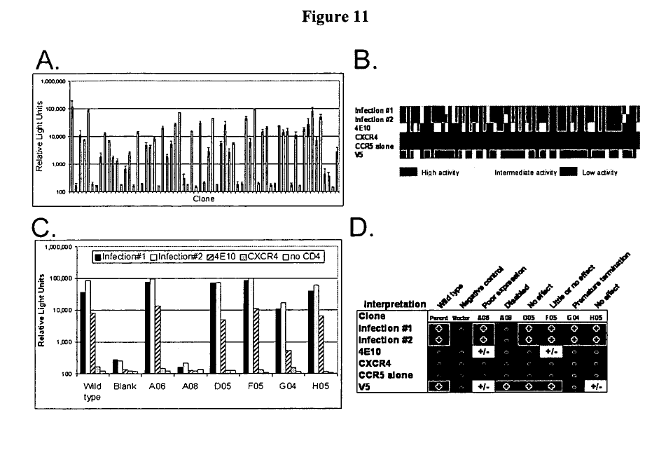

While maxi-prep DNA is often the standard for preparation of high quality DNA suitable for transfection of eukaryotic cells, miniprep DNA is more convenient for small-scale preparations of DNA from large libraries. To assess whether miniprep DNA is of sufficient quality for reverse transfection, DNA encoding a variety of fluorescent proteins was prepared using two different commercial miniprep kits (Qiagen spin column minipreps and Promega Wizard SV 96 Plasmid DNA purification system), and reverse transfected into HEK-293 cells. DNA prepared using a commercial maxiprep kit (Qiagen maxiprep) was used as a positive control. Different concentrations (100 ng or 200 ng) of plasmid was arrayed in microplate format, as outlined herein, with different volumes (300 nl or 500 nl) of transfectant (Lipofectamine 2000), with or without the following additives: 1% sucrose, 1% glycerol, 1% DMSO, 0.04% gelatin. Reverse transfection was evaluated by measuring the efficiency of reverse transfection using a range of DNA, Lipofectamine 2000 transfectant, and stabilizer concentrations (FIG. 5). Results indicated that miniprep DNA could perform nearly as well as maxiprep DNA. The results demonstrated that 100 ng of DNA and 0.3 μl of Lipofectamine 2000 were sufficient to achieve 70-90% transfection efficiency, and that while some additives (e.g. sucrose) had beneficial effects, none were required. Many different proteins have been successfully expressed in cells by reverse transfection (FIGS. 5, 11, and data not shown).

Several types of transfectant were tested for reverse-transfection of HEK-293, QT6, and BHK cell lines. Transfectants included Lipofectamine 2000, Lipofectamine Plus, Lipofectamine, Effectene, Transfectin, and Cytopure. No DNA and DNA alone were also included for comparison. The transfectants were ranked according to transfection efficiency of GFP after visualization of fluorescence in cells, in the order Lipofectamine 2000>=Lipofectamine Plus>Lipofectamine>Transfectin>Cytopure>Effectene>DNA alone=No DNA. Wells with DNA alone or no DNA had few or no fluorescent cells. The rank order of transfectant was equivalent in all three cell types. Other cell types, including HeLa, dog CF2TH, and hamster CHO cells have also been reverse-transfected with success.

### Example 9

### Identification of Amino Acids Contributing to Monoclonal Antibody Binding Sites in CCR5

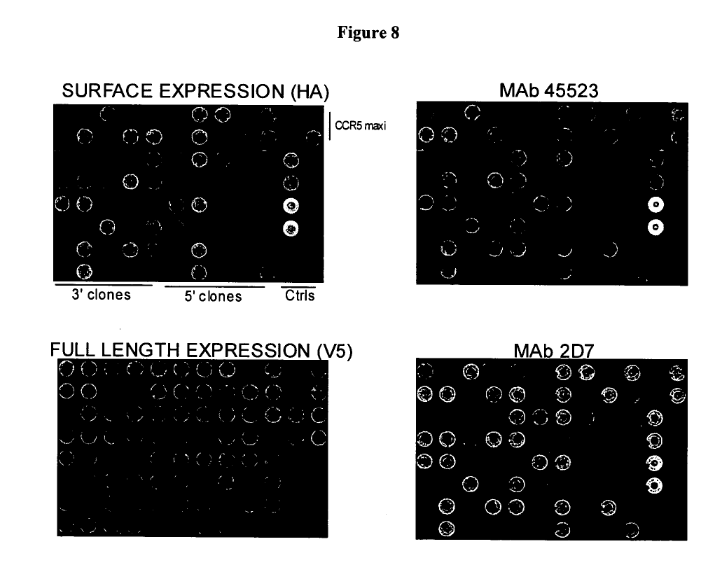

Many different anti-CCR5 MAbs have been generated (Lee, et al. (1999), J. Biol. Chem., 274:9617-9626, Olson, et al. (1999), J. Virol., 73:4145-4155, Wu, et al. (1997), J. Exp. Med., 186:1373-1381), but the epitopes for most of these have been characterized using only a handful of CCR5 mutations, if at all. The monoclonal antibodies 2D7 and 45523 recognize conformation-dependent epitopes of CCR5; their ability to bind the protein is dependent upon a normal tertiary structure at the epitope site, and loss of antibody reactivity to a particular CCR5 mutant would indicate mutation and disruption of this tertiary structure. By characterizing the ability of conformation-dependent antibodies to bind multiple CCR5 clones mutated at different amino acid positions, these epitope structures can be identified and “mapped”. A ACCR5 mutation expression array was constructed using HEK-293 cells and randomly mutated versions of CCR5, as outlined herein. Reactivity of the conformation-dependent antibodies 2D7 and 45523 were tested by immunofluorescent staining of transfected cells (FIG. 8). Reactivity indicates preservation of the antibody's epitope, while loss of reactivity suggests mutation of the epitope. Cells expressing wild-type CCR5, and previously characterized mutant CCR5 proteins (CCR5/CCR2b chimeras 5222 and 2555, CCR5/CCR1 chimera 5111, and N-terminal truncation CCR5A16) were used as controls (on the plate shown and on other plates). Full-length surface expression of each mutant was confirmed using anti-HA (N-terminal tag) and anti-V5 (C-terminal tag, detected in permeabilized cells) antibodies.

### Example 10

### Epitope Mapping of Monoclonal Antibodies to Env

Thousands of monoclonal antibodies and human Fabs against Env have been generated and characterized over the past two decades. However, only a handful are known to be broadly cross-reactive and neutralizing, and only a small subset have been well mapped to determine their precise epitopes (Wyatt, et al. (1998), Nature, 393:705-711, Wyatt, et al. (1998), Science, 280:1884-1888). The most useful MAbs against Env have generally targeted conserved domains and conformational epitopes of the protein. In contrast, MAbs that target variable loops and linear epitopes typically are type-specific and often of little use. The ability of the cell array to quickly and efficiently map such epitopes could enable better MAbs to be identified. A collection of several dozen antibodies raised against gp120, gp140, and oligomeric Env (gp140) that has been well-characterized (Broder, et al. (1994), Proc. Natl. Acad. Sci. USA, 91:11699-11703, Earl, et al. (1994), J. Virol., 68:3015-3026) is used. This library contains antibodies directed against linear epitopes, conformation-dependent epitopes (D20, D33, T3, T4, T6, T9, T10), gp120 (D20, D33), gp41 (D12, T3, T4, T6, T9, T10), oligomers, and monomers. Each antibody is hybridized with an Env mutation array, and visualized using a fluorescently-labeled secondary antibody. Reactivity (or lack of reactivity) of each MAb is compared to reactivity of a polyclonal sera and of a C-terminal epitope tag that verifies protein expression.

Epitopes are broadly classified according to subunit (gp120 or gp41, as determined by control spots expressing only gp41 or gp120), domain (variable or conserved regions, as determined by control spots lacking variable loops 1, 2, and/or 3), and conformation (linear or conformational, as determined by denaturation prior to hybridization). Denaturation of Env on the cell array is achieved by blotting of the array onto a nylon filter with treatment similar to a Western blot (Ziauddin, et al. (2001), Nature, 411:107-110). Alternatively, arrays are fixed with methanol and treated with DTT. Results from the cell array are expected to mirror epitope maps of MAbs previously identified.

### Example 11

### Identification of Amino Acids Involved in Ligand Binding

Binding of a number of chemokines by CCR5, including MIP-1α, MIP-1β, and RANTES, is known to be associated with important physiological processes such as cellular migration. Ligand binding is a conformation-dependent event, and reductions in ligand binding ability by CCR5 mutants could result from loss of amino acids with important contact functions, or with important influences on the local or global structure of the ligand binding site. A ACCR5 mutation expression array is constructed using HEK-293 cells as outlined herein. Full-length surface expression of each mutant is confirmed, and protein expression standardized, using anti-V5 and anti-HA antibodies. The ability of each ACCR5 to bind MIP-1α is assessed using radiolabeled MIP-1α (Perkin-Elmer). ¹²⁵I-MIP-1 (0.05 nM) is added to each well, and allowed to bind for 1 hour at 23° C. After removal of unbound ligand by filtration through Whatman GF/C filters and washing with HBS containing 500 mM NaCl, bound ligand is detected by measuring radioactivity in a gamma counter (as described in (Doranz, et al. (1999), J. Virol., 73:10346-10358)). Untransfected cells, cells expressing wild-type CCR5, and cells expressing previously characterized CCR5 mutants with loss of ligand binding are used as controls.

### Example 12

### Identification of Amino Acids Important in Ligand-Mediated Cell Signaling

Upon ligand binding, CCR5 exerts its physiological effects by stimulating a number of intracellular signaling events, including Ca+2 release from intracellular stores. The ability of each mutant to stimulate increases in cytosolic Ca+2 concentration is tested using a ratiometric calcium indicator, Fura-2. The AM-ester form of Fura-2 is cell membrane permeable. Upon entering the cytoplasm, the AM-ester group is removed by endogenous esterases, trapping the indicator in the intracellular space. Upon binding calcium, Fura-2 undergoes an increase in fluorescence emission. A ΔCCR5 mutation expression array is constructed using HEK-293 cells as outlined herein. Cells expressing each ACCR5, and control cells, are loaded with Fura-2-AM, and then stimulated with MIP-1α (60 nM) (as described in (Doranz, et al. (1999), J. Virol., 73:2752-2761)). Changes in fluorescence emission, indicating fluctuations in cytosolic Ca²⁺ concentration indicative of CCR5 cell signaling will be monitored in a Molecular Devices FlexStation. A comparison of mutated CCR5 clones unable to signal upon exposure to MIP-1α, and those unable to bind the ligand, will facilitate identification of amino acids specifically involved in the intracellular signaling mechanism. Alternative ligands (MIP-1β, RANTES, MCP-2) could also be used.

### Example 13

### Identification of Sites of Interaction between HIV-1 Env and CCR5

Previous work has demonstrated that strains of HIV-1 and SIV differ in how they interact with CCR5 during fusion (Doranz, et al. (1997), J. Virol., 71:6305-6314, Edinger, et al. (1997), Proc. Natl. Acad. Sci. USA, 94:4005-4010, Rucker, et al. (1996), Cell, 87:437-446), that these sites can change in response to CCR5 inhibitors (Kuhmann, et al. (2004), J Virol, 78:2790-807, Trkola, et al. (2002), Proc Natl Acad Sci USA, 99:395-400), and that these sites can be correlated with viral pathogenicity (Cayabyab, et al. (1999), J Virol, 73:976-84, Edinger, et al. (1997), Proc. Natl. Acad. Sci. USA, 94:4005-4010, Karlsson, et al. (1998), J. Exp. Med., 188:1159-1171). The ability of the CCR5 Mutation Array to identify amino acids that contribute to coreceptor function is assayed using a previously employed virus infection assay (Doranz, et al. (1999), J. Virol., 73:10346-10358, Doranz,. et al. (2001), AIDS Res Human Retroviruses, Edinger, et al. (1997), Proc. Natl. Acad. Sci. USA, 94:4005-4010). Briefly, HIV-1 containing the JRFL strain of Env and carrying a luciferase reporter gene is used to infect a Mutation Array expressed in HEK-293 cells. Cells that express permissive versions of CCR5 are expected to permit HIV-1 entry, as detected by luciferase expression, while mutant versions of CCR5 that no longer support HIV-1 infection are identified by lower or no luciferase expression. The HEK-293 cells used in this assay do not normally express CCR5 but will constitutively express CD4 (necessary, but not sufficient for fusion). Controls, including an HIV-1 strain (NL-43) that uses an alternate coreceptor (CXCR4), will also be tested.

### Example 14

### Use of a cDNA Library Expression Array to Identify Novel Genes

In addition to using arrayed plasmids for functional and structural screening of large libraries of mutated variants of a single gene, the cell array can also be used to screen other types of libraries, such as tissue-specific mammalian cDNA expression libraries. A cell-line that does not normally support HIV-1 infection is used to express an arrayed mammalian cDNA library, and will be screened for novel HIV co-receptors. cDNA from a human brain mammalian expression library (Clontech) is prepared, divided into 96 sub-fractions, and arrayed in individual wells of a microplate, as outlined herein. Canine CF2TH cells constitutively expressing human CD4 are added to each well and reverse transfected as described herein. The expression vector used in the library is pEXP1, which drives cDNA expression from a human CMV promoter/enhancer, and which contains the internal ribosome entry site of the encephelomyocarditis virus to ensure high level mRNA translation. After 24 hours in culture, cells are screened for their ability to support HIV-1 infection using a previously employed virus infection (HIV-luciferase) assay ((Doranz, et al. (1999), J. Virol., 73:2752-2761)). A number of strains of HIV-1 are individually tested, including JRFL, NL43, and 89.6. The inclusion of a cDNA coding for functional HIV-1 co-receptors in each library fraction is inferred from the presence of virus (luciferase expression) within the cells expressing that fraction.

One skilled in the art would recognize that alternative libraries, including libraries of peptides (such as random oligomers) could be used for this or other screens, that the library could be divided to contain any desired number of cDNA representatives in each fraction, and that any type of plate or microarray format could be chosen. One skilled in the art would recognize that alternative libraries and vectors, including viral vectors, could also be used to create the expression array. One skilled in the art would recognize that such mammalian library expression arrays could be used to identify novel genes for a variety of substrates, ligands, and functions.

### Example 15

### Kinetic Analysis of Cell Array Expression

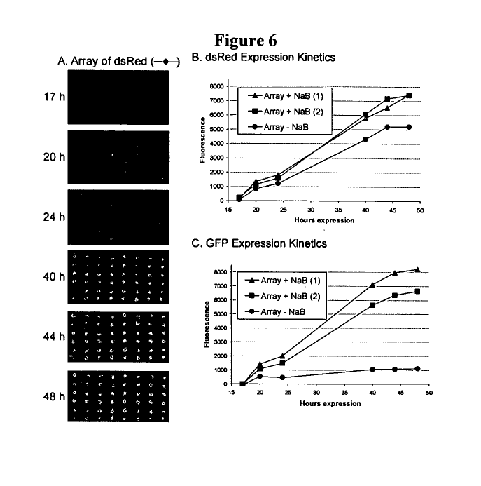

The ability to obtain quantitative kinetic expression data for arrayed cDNA would enable screening of libraries containing promoter or transcription element variants for dynamic expression or functional studies. DNA from plasmids containing the fluorescent reporter proteins GFP or dsRED, the expression of which are driven by a CMV promoter/enhancer, was prepared and arrayed in microarray format, as described herein. HEK-293 cells were added, cultured for 48 hours, and visualized at intervals using an AlphaArray 7000. FIG. 6A depicts the direct visualization of cell fluorescence, while FIG. 6B is a graphical representation of the expression of fluorescence for the entire set of images measured over time. FIG. 6C depicts a similar graphical representation of expression of GFP using the same cells and expression vector. Significant expression of each fluorescent protein was detected by 20 hours, reaching an apparent maximum at 48 hours. In both cases, the effect of adding the promoter-enhancer, sodium butyrate (NaB), after 20 hours of culture was examined, and is demonstrated in FIGS. 6B and 6C.

### Example 16

### Mapping of a Promoter Region

In addition to functional and structural screening of transcribed gene regions (i.e. proteins), transcription regulatory sequences for genes can also be screened in array format, when linked to reporter sequences, such as fluorescent or luminescent proteins. A random mutation library of the CMV promoter (ΔCMVp), linked to a luciferase reporter gene, is created, arrayed, and expressed, as outlined herein. Briefly, a plasmid containing the CMV promoter, flanked by unique restriction sites, and directly upstream of a luciferase reporter gene, is created. The CMV promoter is subjected to random mutagenesis (Clontech Diversify kit), excised using the unique restriction sites, and ligated back into the parental vector. ΔCMVp plasmid is prepared, arrayed in the wells of a 384-well plate, and reverse transfected into HEK-293 cells. Cells are grown in culture, with or without the promoter-enhancer sodium butyrate, for 24 hours, after which they are lysed and tested for luciferase expression. Cells reverse transfected with wild-type CMV-luciferase cDNA will be used as a positive control. One skilled in the art would recognize that other transcription elements such as enhancers, inhibitors, repressors, and other locus control region elements could be screened using a similar strategy.

### Example 17

### Mapping of Drug Binding Sites

In addition to screening for the ability of mutant proteins to bind natural or endogenous ligands, libraries of mutant proteins could also be used to characterize the structural basis for binding of exogenous ligands and drugs to receptors and other proteins. A library of ΔCCR5 variants will be created, arrayed into plate wells, and reverse-transfected into HEK-293 cells, as described herein. Full-length surface expression of each mutant is confirmed, and protein expression measured, using anti-HA and anti-V5 antibodies. The ability of each ΔCCR5 to bind an anti-CCR5 drug, TAK779, is assessed using radiolabeled TAK779. TAK779 is added to each well, and allowed to bind for 1 hour at 23° C. After removal of unbound TAK779 by filtration through Whatman GF/C filters and washing with HBS containing 500 mM NaCl, bound ligand is detected by measuring radioactivity. Untransfected cells and cells expressing wild-type CCR5 are used as controls. One skilled in the art would recognize that other exogenous ligands and drugs, other detection methods, and other receptors or proteins could be screened using similar methods. Mapping of the drug binding site could be performed for GPCRs, ion channels, transporters, enzymes, and other drug targets.

### Example 18

### Mapping Intracellular Signaling-Partner Binding Motifs

In addition to screening for ligand-binding by receptors or other proteins, arrayed mutant libraries can also be screened to identify the structural basis of receptor association with intracellular signaling-partners such as kinases, G-proteins, and arrestins. A CXCR4 mutant library is constructed as outlined herein. The CXCR4 library is arrayed in microplate wells and reverse-transfected into cells stably expressing a β-arrestin-GFP fusion protein (Transfluor, Norak Biosciences Inc). Full-length expression of each mutant is confirmed, and protein expression measured using anti-V5 and anti-HA antibodies. The ability of each ΔCXCR4 to interact with β-arrestin-GFP is assessed using the ligand SDF-1α. SDF-1α (50 ng) will be added to each well, and the fluorescent pattern of cells monitored in real time using a Cellomics KineticScan. Cells in which β-arrestin is not interacting with receptors will exhibit diffuse cytoplasmic fluorescence, while those in which β-arrestin binds the signaling regions of CXCR4 will develop a distinctly punctate fluorescent pattern as the fusion protein associates first with cell membranes, then with endocytic vesicles. Untransfected cells, and cells expressing wild-type CXCR4 are used as controls. A mutation library focused on the cytoplasmic domains of CXCR4, such as the C-terminal tail, can also be used for focused analysis of known regions of interest.

### Example 19

### Mapping Protein Trafficking Motifs

Vpr is an HIV-1 protein important for viral trafficking to the nucleus (Le Rouzic, et al. (2002), J Biol Chem, 277:45091-8). A library of vpr mutants (Δvpr) is created as described herein. The vpr protein comprises a cytoplasmic V5 tag for immunofluorescent localization. The mutation library is reverse transfected into HEK-293 cells as described herein. Untransfected cells, and cells transfected with a wild-type vpr-V5 gene are used as controls. Full length expression of each Δvpr will be confirmed, and protein expression measured in permeabilized cells using anti-V5 antibodies and immunofluorescent staining. The ability of each Δvpr to localize to and interact with the nuclear envelope will be monitored by immunofluorescent staining and localization within the cell. Other markers of the nucleus and other cellular organelles can also be co-localized, as desired, using other fluorescent markers such as Hoechst staining of nucleic acids.

### Example 20

### Mapping of a Secreted Protein

The mutation array can also be used for high-throughput structural analyses of secreted proteins such as cytokines, chemokines, or other extracellular fluid constituents. A mutation library of the chemokine RANTES (ΔRANTES) is arrayed within microplates and used for screening structural interactions with its cognate receptor, CCR5. Briefly, random mutations of the gene coding for RANTES are cloned into pcDNA3, arrayed into microplate wells, and reverse transfected into HEK-293 cells as described herein. Untransfected cells, and cells transfected with wild-type RANTES cDNA are used as controls. Culture medium supernatant is collected after 48 hours. Full length expression of each ΔRANTES is confirmed by western blot using an anti-RANTES antibody. Supernatant containing each ΔRANTES is added to microplate wells containing HEK-293 cells expressing wild-type CCR5, and which have been pre-loaded with the fluorescent calcium reporter Fura-2-AM. The ability of each ΔRANTES to interact with CCR5 and stimulate receptor activation will be assessed by measuring fluorescence in each well while stimulating with each ΔRANTES, indicating release of Ca⁺⁺ from intracellular stores. One skilled in the art would recognize that a similar approach could also be used to identify enhanced or diminished binding, agonist, or antagonist properties of exogenous CCR5 (or other protein) ligands such as drugs, peptides, and antibodies.

### Example 21

### Mapping of Ion Channel Functional Regions

Potassium ion (K⁺) channels have been particularly well studied due to their primary importance in excitable cells (Ford, et al. (2002), Prog Drug Res, 58:133-168). A number of toxins are known to bind specifically to K-channels, bacterial K-channels have been crystallized, and a variety of K-channels with various regulatory features have been defined and characterized. Drosophila Shaker (GenBank Accession Number M17211, GI: 157063) is a voltage-regulated (opens upon depolarization) K-channel that serves as a prototype ion channel herein due to its extensive characterization. Mutation expression array libraries can be used to screen the structural features responsible for the function of ion channels such as Shaker, Kv1.3, nAChR, KCNH2, and CFTR. A mutation library containing mutant Shaker (AShaker) variants with substitutions at every amino acid position, is produced, arrayed in microplate wells, and reverse transfected into HEK-293 cells as described herein. The ability of each ΔShaker to function by allowing traversal of K⁺ across the cell membrane will be assessed. Briefly, the membrane potential probe FMP (Molecular Devices) is added to cells to a final concentration of lx. A high K-buffer (30 mM K2SO4 final concentration) will then be added to wells using a Molecular Devices FlexStation. ΔShaker variants that retain voltage-regulated functionality will open in response to depolarization and a change in fluorescence of FMP dye (ex530/em565) will occur. Untransfected cells, cells transfected with wild-type Shaker or an inactivation-gate removed variant of Shaker, and pre-incubation with inhibitory toxins are used as controls.

### Example 22

### Mapping of Transporter Functions

A library of mutants of the amino acid transporter MCAT-1, containing substitutions at every amino acid position (ΔMCAT-1) are created, prepared, arrayed, and reverse-transfected into HEK-293 cells, as described herein. The ability of each ΔMCAT-1 to function in amino acid transport will be assessed using radioactively labeled amino acids. Briefly, radioactively-labeled amino acids, and the necessary co-factors for MCAT-1 function, are added to wells containing cells expressing ΔMCAT-1 variants, and cultured for 1 hour at 37° C. Free amino acids are removed by washing with buffer, and incorporated amino acids evaluated by measuring the radioactivity remaining in the cells. Untransfected cells and cells transfected with wild-type MCAT-1 will act as controls. One skilled in the art would recognize that other types of detection could also be used, such as the incorporation of fluorescent amino acids.

### Example 23

### Mapping of Kinase Function

The cellular effects of many single-transmembrane receptors such as platelet-derived growth factor receptor (PDGFR) and epidermal growth factor receptor, are mediated by the actions of intracellular kinases such as AKT, JNK and MEK. A mutation expression array is used to identify the structural bases for the function of the serine/threonine kinase AKT. A library of mutants of human AKT, containing substitutions at every amino acid position (ΔAKT) is created, prepared, and arrayed in plate wells, as described herein. ΔAKT plasmid is reverse-transfected into murine 3T3 cells stably expressing human PDGFR. The ability of each ΔAKT to phosphorylate a substrate upon appropriate stimulation of PDGF is assessed using a homogenous colorimetric reporter assay (IQ Kinase Assay, Pierce). Briefly, after 24 hours in culture, cells are stimulated for 30 minutes with 100 ng/ml PDGF, and then lysed. After washing, IQ reagents are added (including dye-labeled peptide substrates), and kinase activity assessed by measuring fluorescence in a fluorometer. ΔAKTs that do not exhibit normal kinase function could result from loss of receptor interaction, or loss or enzyme activity.

### Example 24

### Iterative Screening of Mutation Arrays

During screening of single amino acid substitution mutants, a variant may be identified for which it is desirable to explore the effect of further, additive mutations. For example, if a particular mutation increases the affinity of a receptor for a ligand, it may be desirable to investigate whether further simultaneously-occurring mutations may have an additive effect. However, it is impractical for all but the smallest proteins, to assemble mutation libraries where every amino acid position is substituted by every possible amino acid. When combinations of more than one amino acid substitution are considered, the potential number of mutants becomes exponentially larger. To overcome this problem of scale, an iterative approach can be adopted, where mutant proteins are selected on the basis of a desired characteristic, and a ‘second generation’ mutation library produced from these selected ‘first generation’ mutants. In this way, mutations resulting in a cumulative effect can be identified.

For example, X4-strains of HIV-1 use CXCR4, but not CCR5 as a co-receptor. A mutation library of ΔCCR5 variants is produced, arrayed and reverse transfected as described herein. The ability of X4 strains to infect cells expressing these ΔCCR5s is assessed as described, and the ACCR5 exhibiting greatest ability to act as a co-receptor selected to form the basis for an additional round of mutagenesis, reverse transfection, and functional screening. One skilled in the art would recognize that this process could be continued as many times as desired, and that any alteration to a genetic sequence could be randomly or selectively introduced into a cDNA sequence prior to its utilization in mutation library production.

### Example 25

### Functional Rescue of Activity

Human CCR5 is a major co-receptor for HIV-1. However, murine CCR5 (mCCR5) is unable to act as a co-receptor for HIV. A mutation expression array of mCCR5 is used to assess the structural bases for this functional difference between it and human CCR5. A mutation library of ΔmCCR5 variants is produced, arrayed and reverse transfected into murine 3T3 cells as described herein. The 3T3 cells constitutively express CD4. The ability of each ΔmCCR5 to act as a co-receptor for the JRFL strain of HIV-1 is assessed using a viral infection assay, as described herein. Briefly, replication-incompetent HIV-1 containing a luciferase reporter gene is added to arrayed cells expressing each ΔmCCR5, and viral infection is evaluated by lysing cells 2 days post-infection and performing a luciferase assay.

### Example 26

### Functional Rescue of HIV Budding

Murine cells do not support HIV-1 viral assembly and budding. An HIV-1 Gag mutation expression array will be used to identify molecular structures responsible for conferring assembly competence to HIV-1 in murine cells. A mutation library of ΔGag variants is produced and arrayed in plate wells as described herein. ΔGag cDNAs are reverse transfected into murine 3T3 cells. One day later, the cells are infected with an HIV-1 virus that is unable to express a functional Gag gene. The ability of each ΔGag to support viral assembly and budding is evaluated by assaying culture supernatant for released virus using RT-PCR. One skilled in the art would recognize that alternative assays for viral presence in the culture supernatant could also be used, including ELISA for components of the structural cores such as p24.

### Example 27

### RNAi Arrays

Double-stranded (ds)-RNA is known to suppress gene transcription in a highly sequence-specific manner. In a process known as RNA interference (RNAi), small segments (21-23 base pairs) are taken up by RNA induced silencing complexes (RISCs), which possess a nuclease activity that degrades endogenous mRNA which exactly matches the dsRNA incorporated in the RISC. When harnessing the properties of dsRNA for RNAi in the laboratory, targets must not only be selected rationally, but also tested empirically, as RNAi efficiency can vary greatly from target to target. A library of CCR5 dsRNA fragments is constructed, and arrayed for screening of RNAi activity and efficiency. Briefly, a library of 21 bp dsRNA fragments of CCR5 will be synthesized, in which each fragment is 1 bp further downstream from the preceding one. Each dsRNA fragment is arrayed in microplate wells, and reverse transfected into HEK-293 cells stably expressing CCR5-GFP as described herein. The ability of each dsRNA fragment to inhibit RNA expression is evaluated by detecting fluorescence using an AlphaArray 7000 fluorometer.

### Example 28

### Diverse Chemokine Receptor Set

There are approximately 20 known GPCR chemokine receptors, and about 50 described chemokines. An expression array in which all 20 chemokine receptors could be simultaneously interrogated against all known chemokines would greatly facilitate high-throughput screening for pairing of orphan ligands and identification of potential agonists and antagonists. Plasmids containing cDNA for all 20 chemokine receptors are constructed, prepared and arrayed in microplate wells in triplicate, as described herein. The cDNAs are reverse-transfected into HEK-293 cells, and the ability of each known chemokine is assessed (individually) for its ability to induce a Ca+2 flux (using a Fura-2 fluorescence assay, as described (Doranz, et al. (1999), J. Virol., 73:2752-2761)). Orphaned ligands, or other chemical agents could also be tested. Cells not transfected with any cDNA, or transfected with a control cDNA that does not code for a GPCR chemokine receptor are used as negative controls. One skilled in the art would recognize that similar arrays could be constructed for any grouping of proteins, related or unrelated, for which simultaneous interrogation of a particular structural quality or function is desirable.

### Example 29

### Diverse Env Set

The HIV envelope protein (Env) is a polyprotein comprised of the glycoproteins gp120 and gp41, responsible for CD4-mediated entry into T4 lymphocytes and monocytes/macrophages, and for at least some of the cytopathic effects of viral infection. Variation in the structure of HIV Env is at least partially responsible for strain-specific virulence and pathogenicity, and plays an important part in determining treatment strategies. An expression array is constructed, in which one-thousand clade-, type-, and strain-specific Env variants are expressed for simultaneous interrogation. Variant Env plasmids are prepared, arrayed in duplicate in the wells of 1536-well plates, and reverse transfected into HEK-293 cells as described herein. Env expression is verified and standardized in each well using human sera reactive against HIV Env. Untransfected cells will act as negative controls.

### Example 30

### Antigenic Characteristics of HIV-1 Env Mutants

A library of nearly two hundred HIV-1 strain MN Env mutations was arrayed in microarray format and tested for 1) retention of structure and 2) retention of function. When detected with a MAb against an epitope tag on the C-terminus of the protein (V5 epitope), Env could be detected throughout the array, confirming the presence of full length clones at nearly all locations. When detected with human sera against HIV-1, Env could be detected, indicating the presence of Env epitopes. When detected with a MAb (2G12) against the HIV-1 Env protein (Trkola, et al. (1996), J. Virol., 70:1100-1108), Env could be detected. Finally, when the binding of a soluble receptor (sCD4-IgG) was assessed, binding was seen throughout the array, indicating that Env had retained the ability to bind to its primary receptor. Several clones that failed to react with sera or bind sCD4 also failed to react with V5 MAb, suggesting that these clones most likely either do not have any insert, or contain a premature stop codon. For example, clone MN15 was sequenced, contains a stop codon, and did not react in any assay.

Similarly, cell arrays in microplate format were used to characterize the amino acid residues that contribute to the antigenic phenotypes of HIV-1 Env (gp160). An HIV-1 MN Env mutant cDNA library was constructed as outlined herein. 88 of these mutants, and 8 controls (including wild-type Env, GFP, and vector alone) were arrayed, and expressed by reverse transfection in HEK-293 cells. After 24 h, cells were fixed in 0.5% paraformaldehyde, and protein expression analyzed by immunofluorescence using multiple antibodies: 2F5 and 2G12 recognize gp120 on the cell surface, and anti-V5 recognizes a mutation-exempt C-terminal tag on gp160 (and indicates full translation of the protein, as detected after methanol fixation). Nearly all clones contained a fully-translated gp160, as indicated by detection of V5 at the C-terminus of gp160. Most, but not all, of the clones that were fully translated also trafficked to the cell surface and were recognized by 2F5 and 2G12.

### Example 31

### Identification of Potential Env Immunogens

The purpose of studying HIV-1 Env cell arrays for vaccine application is to identify antigens that can elicit a potent humoral immune response. Even for the most potent antibodies against HIV-1 Env, the immunogens that gave rise to these antibodies are largely unknown. Studies indicate that genetic modification of Env can result in enhanced immunogenicity. For example, Reitter et al. showed that elimination of just 2 N-linked carbohydrate addition sites in the gp120 protein of SIV Env resulted in a virus that, when used to infect rhesus macaques, elicited neutralizing antibodies that could inactivate the parental, fully glycosylated virus (Reitter, et al. (1998), Nature Med., 4:679-684). These modifications make SIV Env relatively independent of CD4, with the result being that the virus can infect cells by directly interacting with the CCR5 coreceptor (Hoffman, et al. (1999), Proc. Natl. Acad. Sci. USA, 96:6359-6364). The mechanism underlying this phenotype appears to involve enhanced exposure of the conserved coreceptor binding site in gp120. Other studies have found that deletions in the V1/V2 region and in other regions of Env can enhance immunogenicity (Kolchinsky, et al. (2001), J. Virol., 75:3435-3443).

The goal of this experiment is to utilize an Env cell array to test whether better immunogens can be identified using this approach. Experimental analysis includes hybridization of monoclonal antibodies to randomly mutagenized Env cell arrays. Antibodies to be assessed will include the broadly cross-reactive and neutralizing antibodies F105, 2F5, b12, D20, 17b, 48d, 2G12, and T4. Env mutants that react strongly with a given MAb indicate a mutation in Env that enhances important properties of its immunogenicity. For example, a mutant that reacted more strongly with the antibody 17b would likely result from exposure of the normally hidden coreceptor binding site, and would also likely exhibit CD4-independence in functional assays (Hoffman, et al. (1999), Proc. Natl. Acad. Sci. USA, 96:6359-6364, Kolchinsky, et al. (2001), J. Virol., 75:2041-2050, Kwong, et al. (1998), Nature, 393:648-659, Wyatt, et al. (1998), Nature, 393:705-711). Similarly, Envs that react with the MAb 2F5 will likely be the result of mutations that unmask a hidden epitope within the C-terminus of the gp41 ectodomain (Muster, et al. (1993), J. Virol., 67:6642-6647). Mutations identified with this approach will be sequenced and reintroduced into the wild type Env by site-directed mutagenesis (Quickchange kit, Stratagene) to confirm and isolate the affects of different mutations. The forms of Env that react most strongly with neutralizing antibodies are promising immunogens that could, if put into animals, elicit similar effective antibodies.

### Example 32

### Functional Analysis of HIV-1 Envelope

The natural function of Env involves 1) binding the cellular receptor CD4, 2) binding a cellular coreceptor, typically either CCR5 or CXCR4, 3) changing conformation that results in exposure of a fusion peptide, and 4) mediation of fusion between the viral and cellular lipid bilayers (Doranz, et al. (1999), J. Virol., 73:10346-10358). During infection of some cell types, such as dendritic cells, binding to another cellular receptor (DC-SIGN) is also involved. Each of these steps can be used as measurements of Env function. In addition to direct measurement, these functions can also be blocked with reagents (e.g. MAbs, seras, proteins, and chemical inhibitors).

The ability of Env to bind a receptor can be separated from the ability of Env to induce membrane fusion. In order to measure binding of CD4 to Env, a soluble version of CD4 containing a rabbit-IgG fusion domain will be used (sCD4-IgG). Detection is measured using a fluorescently-labeled secondary antibody against the Fc region of the CD4 fusion protein. In order to measure binding of Env with coreceptors, fluorescently labeled virus “particles” that contain coreceptors will be used (such particles are termed “pseudotypes”) (Endres, et al. (1997), Science, 278:1462-1464). GFP-tagged coreceptors have been previously incorporated into reverse-pseudotypes and detected by fluorescence microscopy (Doranz, et al. (2003)). Binding of the coreceptors CCR5 and CXCR4 is assessed in the presence of soluble CD4 (sCD4). Binding of DC-SIGN is measured in two ways, 1) with soluble DC-SIGN (using antibodies for detection) and 2) with pseudotypes containing full-length DC-SIGN (fluorescently labeled). An IgG fusion protein will be constructed if necessary. DC-SIGN functions as a homotetramer that is formed only within the context of a lipid bilayer, so presentation of DC-SIGN in its native lipid bilayer (as part of a pseudotype) is expected to more accurately represent its native conformation.

The ability of Env mutation arrays to support membrane fusion is assayed using a cell-cell fusion assay. In this assay, the function of Env is measured by testing the ability of cells expressing Env to fuse with cells expressing receptors. Incorporation of a reporter system provides a quantifiable measurement of fusion (target cells contain a GFP gene driven by a T7 promoter, and complementary “effector” cells contain T7 polymerase driven from a vaccinia vector). This assay has been previously described (Rucker, et al. (1997), Methods in Enzymology: Chemokine Receptors, 288:118-133). Measuring the kinetics of cell-cell fusion using traditional assay techniques has also been done (Rucker, et al. (1997), Methods in Enzymology: Chemokine Receptors, 288:118-133), and such analysis is performed using cell arrays and kinetic detection. Alternative detection systems include dye transfer and fluorescence resonance energy transfer (FRET)-based readouts.

An alternate and complementary method of measuring Env fusion is a virus infection assay. However, because these cell arrays express Env, the virus used to infect the arrays must contain the receptors for Env. Such a virus is termed a “pseudotype.” Pseudotypes carrying a GFP reporter gene are used to infect cell arrays expressing HIV-1 Env, as previously described (Endres, et al. (1997), Science, 278:1462-1464). Upon integration of the virus, the GFP reporter is expressed from a CMV promoter and can be measured, indicating that the Env at that spot can support infection.

### Example 33

### Use of Mutation Arrays for Analysis of Env Functions

In conjunction with antigenic analysis, functional results are used to correlate structure and function across a library of Env mutations simultaneously. The use of different chemokine receptors as coreceptors by HIV-1 has been linked directly to pathogenicity of the virus (Doranz (2000), Emerging Therapeutic Targets, 4:423437). HIV-1 strains that use CXCR4 as a coreceptor are correlated with an increased rate of disease progression, while strains that use CCR5 are preferentially transmitted, the first type of HIV-1 to emerge in newly infected individuals, and representative of the majority of primary strains of HIV-1 found in patients worldwide. Using traditional mutation analysis, coreceptor selectivity has been most closely linked with amino acids in the V3 loop of Env, but the V1 and V2 loops of Env also play a role (Doranz (2000), Emerging Therapeutic Targets, 4:423-437). The crystal structure of HIV-1 gp120 also revealed a conserved but hidden structure that becomes exposed upon gp120 interaction with CD4 that is believed to represent the face of gp120 that interacts with the coreceptors (Rizzuto, et al. (2000), AIDS Res Human Retroviruses, 16:741-749, Rizzuto, et al. (1998), Science, 280:1949-1953). Using the cell array, Env mutations are screened for their ability to 1) bind and 2) fuse using the receptors CD4, DC-SIGN, CCR5, and CXCR4 (interaction with coreceptors requires the presence of CD4 or soluble CD4). The loss (or gain) of ability to bind a receptor would identify the Env structures responsible for these functions. A cell array focusing on the region encompassing the V3 loop can also be analyzed. Results are compared with prior structure-function studies and the crystal structure of gp120 (in complex with sCD4) in order to validate the residues identified by cell array analysis. For some Env interactions (CCR5, CXCR4, and DC-SIGN), only a handful of interacting residues in Env have been previously mapped. Studies can also include previously characterized coreceptor mutants and chimeras (Doranz, et al. (1997), J. Virol., 71:6305-6314, Doranz, et al. (1999), J. Virol., 73:2752-2761, Rucker, et al. (1997), J. Virol., 71:8999-9007, Rucker, et al. (1996), Cell, 87:437-446) to map protein-protein interaction sites on both Env and receptor.

CD4-independence was originally discovered in HIV-2 (Endres, et al. (1996), Cell, 87:745-756), but was soon shown to be a property of many types of SIV and some strains of HIV-1 (Edinger, et al. (1997), Proceedings of the National Academy of Sciences, USA, 94:14742-14747, Hoffman, et al. (1999), Proc. Natl. Acad. Sci. USA, 96:6359-6364, Kolchinsky, et al. (2001), J. Virol., 75:3435-3443). Strains of HIV-1 that do not require CD4 for fusion typically use CXCR4 as their primary receptor, but some strains have been identified or created that use CCR5 as their primary receptor. Interestingly, CD4-independence is thought to immunologically compromise HIV-1 strains by exposing portions of Env that otherwise remain hidden until triggered by CD4. For example, CD4-independent Envs react strongly with broadly cross-reactive antibodies such as 17b that otherwise recognize Env only after it has been triggered into a fusogenic conformation by CD4 binding. CD4-independent Envs that are used to produce antibodies have resulted in antibody responses that are generally more potent against all forms of Env (Hoffman, et al. (2000), Proc. Natl. Acad. Sci. USA, 97:11215-11220, Hoffman, et al. (1999), Proc. Natl. Acad. Sci. USA, 96:6359-6364, Kolchinsky, et al. (2001), J. Virol., 75:2041-2050). Env mutation arrays are used to screen for CD4-independence of Env. Envs are tested for binding, fusion, and infection using CCR5 and CXCR4 pseudotypes (in the absence of CD4) (Endres, et al. (1997), Science, 278:1462-1464). Results are compared with identical assays conducted in the presence of CD4.

Dissociation of gp120 from the gp41 subunit, a process termed shedding, destroys the native structure of Env and presents a less immunogenic form of the protein (Moore, et al. (1990), Science, 250:1139-1142). Extreme temperatures, the addition of soluble CD4, and prolonged receptor contact have all been demonstrated to induce shedding of gp120 from gp41 and alter Env conformation (Doranz (2000), Emerging Therapeutic Targets, 4:423-437, Moore, et al. (1992), AIDS Res Human Retroviruses, 8:443-450, O'Brien, et al. (1994), J. Virol., 68:5264-5269). A vaccine candidate based on Env would maintain structural stability even under adverse conditions, both during manufacture and in vivo. More stable versions of soluble Env have been created by introducing stabilizing amino acids (e.g. Cys) at select locations in Env (Binley, et al. (2000), J. Virol., 74:627-643, Farzan, et al. (1998), J. Virol., 72:7620-7625), but rationally designing a more stable Env remains a challenging goal for vaccine development. To identify such mutations, cell arrays are exposed to a range of temperatures (4-50° C.) for prolonged periods of time, and then probed for protein structural integrity with the use of conformationally sensitive MAbs. Retention of gp160 tertiary structure, gp41 tertiary structure, as well as oligomeric structure is tested using available MAbs and compared to untreated arrays (37° C.). The ability of Envs to continue to support fusion and/or infection can also be measured. Iterative assay of mutations can also be used to obtain Env variants that display even further improved stability.

### Example 34

### Identification of Drug Resistant Strains of HIV

HIV-1 infection is characterized by massive viral replication, with between 1 and 10 billion virus particles generated each day (Levy (1994)). When coupled with the high error rate of retroviral reverse transcriptase, HIV-1 routinely evades pharmaceutical and immune challenges that might otherwise control viremia. Several novel classes of drugs are in preclinical and clinical development that target several aspect of the HIV-1 replication cycle, including viral entry. The efficacy of each new drug will be a function of not only its pharmacokinetic properties, but also of the ease with which virus can escape from the drug through mutation. Thus, new ways of rapidly detecting and characterizing mechanisms of viral resistance are needed.

The cell array can be used as a tool for identifying residues of Env that are responsible for conferring resistance to drugs. The fusion inhibitors T20 and T1249 (Kilby, et al. (1998), Nat. Med., 4:1302-1307, Wild, et al. (1994), Proc. Natl. Acad. Sci. USA, 91:9770-9774) are being developed by Trimeris/Roche, and T20 (Fuzeon) has entered the market (Kilby, et al. (1998), Nat. Med., 4:1302-1307). Drug resistance to T20 has already been identified in patients, and at least three residues in the HR1 domain of gp41 (GIV) are implicated in this resistance. Identifying residues that confer resistance to entry inhibitors provides not only clinically useful information, but may also guide future development of antiviral compounds early in their development. Analysis of the anti-viral humoral response of patient sera could be analyzed using a similar strategy (assuming that the number of mutants that evade this neutralization may be indicative of the breadth of the sera's neutralizing capability).

The Env mutation array can be applied to the study of several different entry inhibitors including the fusion inhibitors T20 and T1249 (that target Env directly), as well as several inhibitors that target HIV coreceptors (TAK-779, AMD3100, ALX40-4C). Experimentally, Env cell arrays are incubated in the presence of inhibitor (three different concentrations), and measured using fusion and/or infection assays to identify Envs that are more or less sensitive to the inhibitors. Env mutants with reduced (or increased) sensitivity to the inhibitor would indicate amino acid residues that confer resistance. The frequency of resistance among the mutants on the array may be indicative of the ease by which the Env can evade the inhibitor.

### Example 35

### Screening for Ribozyme Activity

Ribozymes, or catalytic RNA, such as hammerhead, have been widely used in applications for the inhibition of gene expression (for a review see (Le Rouzic, et al. (2002), J Biol Chem, 277:45091-8). Hammerhead can bind and cleave any RNA containing the consensus triplet 5′-NUH-3′, where N can be any nucleotide, and H can be C, U or A. However, the secondary structure of mRNA also plays a vital role in the efficiency of ribozyme function, by determining the accessibility of oligonucleotide binding. The creation of suitably selective ribozymes for individual gene inhibition is an empirical process. A mutation array is used to screen a number of hammerhead variants for gene specificity. A library of ribozyme genes is created as previously described (Lieber, et al. (1995), Mol Cell Biol, 15:540-51). Briefly, a series of ribozyme genes are synthesized as double-stranded oligonucleotides, with different 20 bp CCR5-specific sequences flanking the cleavage domain. Oligonucleotides are cloned into the pAdVAntage vector (Promega) which contains the adenovirus virus-associated I (VAI) RNA gene, arrayed in plate wells, and reverse transfected into HEK-293 cells stably expressing a CCR5-GFP fusion protein, as described herein. Cells are grown in culture for up to 24 hours, and fluorescence monitored using an AlphaArray 7000 fluorometer. The ability of each ribozyme to inhibit CCR5 gene expression is inversely correlated to the fluorescence of each culture. Untransfected cells are used as negative controls (and as a calibrator for ribozyme efficiency).

### Example 36

### Correlation of Structure and Function

The ability to correlate a protein's functional data with a structural basis in the protein, is central to the understanding of protein biology. A mutation array is used to produce mutated variants of the small viral protein, vpr, that can separately be analyzed by x-ray crystallography (or alternatively, NMR). A mutant vpr cDNA library, containing single substitutions of every amino acid represented at every position is produced, arrayed, and reverse-transfected into HEK-293 cells as described herein. Mutants exhibiting diminished or enhanced, activities are crystallized in order to understand the precise structural modifications that led to the functional differences.

### Example 37

### Creation of cDNA Library using Viruses

The reverse transfection technique can be performed using viral vectors instead of plasmids. The CF2TH cell-line that does not normally support HIV-1 infection is used to express an arrayed retroviral mammalian cDNA library, and is screened for novel HIV co-receptors. cDNA from a human brain retroviral library (BD Biosciences), using a pLIB vector including the 5′ MLV LTR promoter, is prepared, divided into 96 sub-fractions, and arrayed in individual wells of a microplate, as described herein. Each well also contains a plasmid expressing VSV Env (to mediate fusion of the virions) and pCGP plasmid encoding MLV Gag-Pol. HEK-293 cells are reverse-transfected with this arrayed library and incubated for two days. Supernatant from the cells is harvested and used to infect canine CF2TH cells constitutively expressing human CD4. After 24 hours in culture, cells will be screened for their ability to support HIV-1 infection using a previously employed virus infection (HIV-luciferase) assay ((Doranz, et al. (1999), J. Virol., 73:2752-2761)). A number of strains of HIV-1 will be individually tested, including JRFL, NL43, and 89.6. The inclusion of a cDNA coding for functional HIV-1 co-receptors in each library fraction will be inferred from the presence of virus (luciferase expression) within the cells expressing that fraction.

One skilled in the art would recognize that alternative libraries, including libraries of peptides (such as random oligomers) could also be used, that the library could be divided to contain any desired number of cDNA representatives in each fraction, and that any type of plate or microarray format could be chosen. One skilled in the art would recognize that alternative libraries and vectors, including other viral vectors such as Adenovirus, could also be used to create the expression array. One skilled in the art would recognize that such mammalian library expression arrays could be used to identify novel genes for a variety of substrates, ligands, and functions.

### Example 38

### Preparation of Infectious Viruses

96 different strains of live HIV-1 virus are individually placed in a 96-well microplate, suspended in DMEM containing 2% sucrose. The microplate is frozen at −80 until ready for use, and then thawed. HEK-293 cells constitutively expressing CD4, CXCR4, and CCR5 are added to each well. In a separate, identical plate, each well also receives an antibody (b12) that is tested for its ability to neutralize that particular HIV-1 strain. The virus is allowed to grow for three days prior to harvest of the media and detection of p24, representing productive growth of the virus in the cells. Strains that are neutralized by the antibody are not expected to grow to wild type levels.

### Example 39

### Printing of Microarrays for Reverse Transfection

A library of plasmids, all prepared to be the same concentration, is prepared for reverse transfection in microarray format, as described. The DNA is prepared with 1% sucrose and 0.2% gelatin, and spotted onto the surface of a polylysine slide. The entire spotting procedure is performed with solutions, reagents, and equipment at 4° C. and 65% humidity. The temperature of the spotting procedure allows the drying process to achieve better uniformity of spots for reverse transfection.

### Example 40

### DNA library Array Preparation

A 96-well microplate is prepared for the reverse transfection of a library of CCR5 mutants, as described herein. 0.2 ug of plasmid DNA in a total volume of 25 ul is placed into each well of the microplate (one mutant per well) to create an array of plasmids. 1% sucrose is included with the DNA (but can be excluded if desired). The microplate is frozen at −80° C. until ready for use. Alternatively, the plasmid DNA is allowed to dry to the surface of the plate prior to storage. When ready for use, the plate is allowed to thaw, and 0.75×10⁴ HEK-293 cells, resuspended in 100 ul of Optimem containing 0.2% Lipofectamine 2000, are added to each well. The Lipofectamine 2000 in solution mixes with the plasmid DNA and binds to the cells. The cells are allowed to adhere to the well surfaces, become transfected with the plasmid, and express CCR5 at 37° C. for 48 hours. The media on the cells is changed after 16 h (but can be left on longer if desired). Protein expression within living transfected cells is visualized by immunofluorescence staining of CCR5. The results will illustrate that CCR5 cDNA can be arrayed, reverse transfected, and expressed without premixing of plasmid, transfectant, or gelatin.

### Example 41

### Subtracted cDNA Library Array

Libraries of double-stranded cDNA are prepared from total RNA harvested from B-cells infected with HIV-1, and uninfected B cells. The infected cDNA library is subtracted against the uninfected library using the PCR-Select cDNA Subtraction Kit (BD Biosciences Clontech), and cDNA libraries of differentially expressed genes within a eukaryotic expression plasmid are generated. As a control, the efficiency of subtraction is analyzed by comparing the abundance of GAPDH cDNA before and after subtraction using gene-specific primers and quantitative real-time PCR. 96-well microplates are prepared for reverse transfection of the differential expression libraries, as described herein.

### Example 42

### Antibody Phenotype Array

A plasmid library of cDNAs coding for a human antibody library is prepared for reverse-transfection into HEK-293T cells, as described herein, and spotted on polylysine-coated glass slides. The antibody library is composed of a heavy and light chain coding sequence, each clone within the library containing a different variable region. The heavy chain in some cases may contain a transmembrane-anchoring domain. In some cases the variable region of each clone has been randomly mutated, while in other cases the variable region contains a sequence of defined antigen specificity. After air-drying of the plasmid-lipid complex to the surface, the plate is stored at −20° C. Alternatively, the preparation can be frozen in solution within microplates and stored in solution rather than dried. When ready for use, HEK-293T cells in 10% DMEM are added to the surface of the slide such that a monolayer of living cells is formed. The cells are allowed to adhere to the surface, become transfected with the location-specific plasmid, and express the encoded antibody at 37° C. for 48 hours. Plasmid expression is detected by staining the cells with an anti-human IgG antibody linked to a fluorescent molecule. The cells at each location are also analyzed for phenotypic changes arising from expression of the transfected antibodies. HEK-293T cells naturally express CXCR4, which can be detected on cell surfaces with an anti-CXCR4 monoclonal antibody (12G5). If the plasmid-encoded antibody reacts with CXCR4, or with any component necessary for its expression, the cells at that location on the array will fail to express CXCR4. CXCR4 expression will also be monitored by infection with a GFP reporter virus made using the Envelope protein from HIV-2 strain VCP, a T-tropic HIV-2 strain that uses CXCR4 as a receptor independently of CD4. If the VCP reporter virus enters the cells, the virus will express green fluorescent protein (GFP). If the antibody at that location in the array blocks CXCR4 (or other proteins required for viral infection), the cells at that location will not express GFP. Monitoring of fluorescence on the array will indicate which cells are capable or incapable of supporting HIV infection.

### Example 43

### Antibody Diagnostic Array

A plasmid library of cDNAs coding for human antibodies is prepared for reverse-transfection into HEK-293T cells, as described herein, and spotted on polylysine-coated glass slides. The antibody library is composed of a heavy and light chain coding sequence, each clone within the library containing a different variable region. The heavy chain in some cases may contain a transmembrane-anchoring domain. In some cases the variable region of each clone has been randomly mutated, while in other cases the variable region contains a sequence of defined antigen specificity. After air-drying of the plasmid-lipid complex to the surface, the plate is stored at −20° C. Alternatively, the preparation can be frozen in solution within microplates and stored in solution rather than dried. When ready for use, HEK-293T cells in 10% DMEM are added to the surface of the slide such that a monolayer of living cells is formed. The cells are allowed to adhere to the surface, become transfected with the location-specific plasmid, and express the encoded antibody at 37° C. for 48 hours. Plasmid expression is detected by staining the cells with an anti-human IgG antibody linked to a fluorescent molecule. The array is also used to detect the binding of an antigen to each location of the array. In one embodiment, each antibody clone in the array is directed against a specific protein from a virus pathogen, with different antibody clones directed to different viral proteins. Binding of a viral antigen to a specific location of the array would indicate the presence of that viral antigen.

In some embodiments, the binding could be detected by using a fluorescent antigen, a radioactive antigen, a biotinylated antigen, a second labeled antibody directed to the viral antigen, or a biosensor. In some embodiments, the biosensor can be based on surface plasma resonance, colorimetric diffraction grating, or acoustic wave stimulation. In other embodiments, the transmembrane-anchored antibodies in the cells can be engineered to stimulate pathways within the cells that then produce a measurable signal, such as a calcium change that can be detected with aequorin, as described (Rider, et al. (2003), Science, 301:213-5).

### Example 44

### RNAi Library

Double-stranded (ds)-RNA is known to suppress gene transcription in a highly sequence-specific manner. In a process known as RNA interference (RNAi), small segments (21-23 base pairs) are taken up by cytoplasmic RNA induced silencing complexes (RISCs), which possess a nuclease activity that degrades mRNA exactly matching the incorporated dsRNA. When harnessing the properties of dsRNA for RNAi in the laboratory, targets must not only be selected rationally, but also tested empirically, as RNAi efficiency can vary greatly from target to target. A library of dsRNA fragments is constructed, the elements of the library corresponding to a multitude of genes, and arrayed for screening of RNAI activity and efficiency. Briefly, a library of approximately 21 bp dsRNA fragments are obtained (Ambion, Silencer pre-designed siRNAs). Each dsRNA clone is arrayed in microplate wells, and reverse transfected into HEK-293 cells as described herein. Phenotypic changes in the cells are assayed as desired, examples of which include apoptotic activity, or specific protein expression patterns, or protein activities. One skilled in the art would recognize that other methods of delivering interfering RNA molecules could also be used.

### Example 45

### Ala-Scan Mutagenesis of CCR5

Using Quickchange (Stratagene), each codon of a CCR5 cDNA expression plasmid is individually changed to code for an alanine (Ala) residue (one change per clone within a library of ACCR5 clones). Naturally occurring Ala codons in the wild type sequence are changed to Glycine. Mutations are introduced only in the area of interest, and the vector sequence, epitope tags, and promoter region are excluded from mutation. Selected clones are sequenced to confirm the introduced mutations and are prepared for reverse transfection in array format as described herein. Phenotypic changes, such as antibody recognition and susceptibility to viral infection, are monitored in reverse transfected cells as desired. One skilled in the art would recognize that any gene could be mutated and characterized in a similar manner.

### Example 46

### Pathogen Protein Array

A 96-well microplate is prepared for the reverse transfection of a library of 48 different pathogen genes, in duplicate, as described herein. 0.2 ug of each plasmid DNA with 1% sucrose in a total volume of 25 ul is placed into each well of the microplate (one plasmid per well) to create an array of plasmids. The microplate is frozen until ready for use. Alternatively, the plasmid DNA is allowed to dry to the surface of the plate prior to storage. When ready for use, the plate is allowed to thaw, and 0.75×10⁴ HEK-293 cells, resuspended in 100 ul of Optimem containing 0.2% Lipofectamine 2000, are added to each well. The Lipofectamine 2000 in solution mixes with the plasmid DNA and binds to the cells. The cells are allowed to adhere to the well surfaces, become transfected with the plasmid, and express the protein at 37° C. for 48 hours. The media on the cells is changed after 16 h.

The cells, now expressing pathogenic proteins in an array format, are fixed with paraformaldehyde. Serum from a patient infected with a pathogenic organism, Dengue virus, is used to probe each of the locations on the array. Because the patient has been infected with Dengue virus, his sera will contain antibodies that react with the pathogen protein at a specific location of cells that express Dengue proteins (e.g. PrM, E-protein, or non-structural proteins). The person's sera will not react with pathogen proteins that he has not been exposed to, whereas other patients exposed to those pathogens would. The reactivity of the patient's sera to the proteins is detected using an HRP-coupled anti-human secondary antibody which can be detected with a luminescent HRP substrate (Pierce, Femto reagent).

### Example 47

### Electroporation of Plasmid Library

A 96-well microplate is prepared for the reverse transfection of a library of CCR5 mutants. 0.2 ug of plasmid DNA with 1% sucrose in a total volume of 25 ul is placed into each well of the microplate (one plasmid per well) to create an array of plasmids. The microplate is frozen until ready for use. When ready for use, the plate is allowed to thaw, and 0.75×10⁴ HEK-293 cells, resuspended in 100 ul of Optimem, are added to each well. The DNA is introduced into the cells using a 96-well electroporator (BTX Molecular Delivery Systems). Alternatively, other devices that work on the principal of electroporation are also available (Excellin Life Sciences, Axon Instruments) (Ho, et al. (1996), Crit Rev Biotechnol, 16:349-62, Huang (2002), Proceedings of International Solid-State Sensor, Actuator, and Microsystems Workshop, 198-201). After transfection, the cells are allowed to adhere to the well surfaces and express CCR5 at 37° C. for 48 hours. Protein expression within the living transfected cells is visualized by immunofluorescence staining of CCR5.

### Example 48

### Array of viruses

96 different Adenovirus vector stocks from an Adenovirus library (Galapagos Genomics) are individually placed in a 96-well microplate, suspended in 25 ul DMEM containing 2% FBS and 1% sucrose. The microplate is frozen at −80° C. until ready for use, and thawed when desired. 0.75×10⁴ HeLa cells are added to each well. The virus is allowed to infect the cells for 24 h. The cells are then tested for expression of a desired protein, CCR5, by immunofluorescence staining. One skilled in the art would recognize that the cells could be tested using any structural, functional, or phenotypic assay that can be tested within cells. In addition, any library of viral vectors, including retrovirus, adeno-associated virus, or adenovirus, could be used to introduce a desired library of genes. The genes may be random cDNAs, selected cDNAs, mutated versions of the same cDNA, or other DNA fragments of interest.

### Example 49

### Fixed Membrane Protein Arrays

The expressed mutant CCR5 array expressing variants of CCR5, is constructed as described herein, and then fixed using 2% paraformaldehyde to preserve cell proteins without destroying their structure. The array is then used to detect CCR5 protein structure by immunofluorescence using the anti-CCR5 antibody 2D7 (recognizes a conformational epitope of CCR5) and detected using a secondary FITC-conjugated anti-mouse antibody. Cell fluorescence will indicate expression of wild-type CCR5 or CCR5 mutants possessing 2D7-recognizable epitopes, while lack of fluorescence will indicate expression of CCR5 mutants that no longer react with the 2D7 antibody.

1% Triton X-100 (or other detergents) could be added during fixation to permeabilize the cells and allow for total cellular protein detection. Other methodologies of cross-linking and fixation could also be used, such as methanol, glutaraldehyde, or formaldehyde. One skilled in the art would also recognize that additional proteins and types of protein libraries could also be analyzed using a similar strategy. For example, an array of fixed GPCRs, as described herein, could be constructed. The analysis of such arrays is not limited to determination of immunofluorescence. One skilled in the art would recognize that any cell based technique adaptable to cells fixed onto a solid support can be used to characterize the structural and functional capabilities of proteins.

### Example 50

### Phage Library Array

The Tomlinson I+J bacteriophage library (MRC HGMP Resource Center) comprises over 200 million different single chain Fv “scFV” fragments cloned in an ampicillin-resistant phagemid vector. An scFv fragment is the antigen recognition portion of a monoclonal antibody (MAb), rearranged into a single polypeptide comprising the V_{H} and V_{L} domains joined by a flexible Glycine-Serine linker. The libraries use a single human framework for V_{H} and Vκ, with side chain diversity incorporated at positions in the antigen binding site. The Tomlinson I+J bacteriophage library is recovered in their host strain bacteria (TG1 E. coli) and expanded with the use of a helper strain of phage (KM13). The library is plated onto LB-agar plates such that individual phage plaques can be selected. Each plaque is expanded within a well of a 96-well microplate in order to recover additional phage containing a single antibody from the original antibody library. Clones are then spotted on a glass slide to form a microarray of phage, allowed to dry, and stored frozen until ready to use. Alternatively, each clone is placed in a solution within a well of a microplate and frozen. When ready for use, a lawn of TG 1 bacteria is grown on the microarray slide or in the microplate wells. Growing bacteria are infected by phage and produce the antibody coded by that phage. When fixed to the surface, the cells expressing that antibody are adhered. Alternatively, the surface of the slide or well can be coated to promote the adhesion of the protein or phage of interest, for example with an antibody that binds phage, an antibody that binds the scFV proteins produced by the phage, or with a nitrilotriacetic acid (NTA) surface that binds His-tagged proteins in the presence of Ni+2.

### Example 51

### Array of Cell Lines

47 different cell lines are arrayed into 96-well plates, in duplicate, and including two wells without cells, which can be used as controls. The cell lines may be growth-arrested at this stage. The cell lines are frozen in 10% fetal bovine serum and 10% DMSO in DMEM media within the 96-well plates by using controlled cooling methods to freeze the cells with minimal cellular death. The cells are frozen at −150° C. until ready for further use. Once ready for use, the cells are allowed to thaw at 37° C. The media is changed 1-4 h after thawing to fresh 10% DMEM. The cells are then used for immunofluorescence to determine the expression of a receptor, CXCR4, on the surface of the cells. The cells can then be used to assess the function of CXCR4 in the different cell lines or what effect the expression of CXCR4 has on a property of a cell, e.g. viability, binding, signaling, and the like.

One skilled in the art would recognize that the types of cells placed into each well could vary. In one embodiment, the cells could be stable cell lines consisting of the same cell type, for example CHO cells, but each line selected to express a different protein, for instance a different member of the GPCR family, or a different variant of the same protein.

### Example 52

### Assessment of Drug Specificity

A 96-well microplate is prepared for the reverse transfection of a library of 48 different GPCRs, in duplicate, as described herein. 0.2 ug of each plasmid DNA with 1% sucrose in a total volume of 25 ul is placed into each well of the microplate (one plasmid per well) to create an array of plasmids. The microplate is frozen until ready for use. Alternatively, the plasmid DNA is allowed to dry to the surface of the plate prior to storage. When ready for use, the plate is allowed to thaw, and 0.75×10⁴ HBEK-293 cells, resuspended in 100 ul of Optimem containing 0.2% Lipofectamine 2000, are added to each well. The Lipofectamine 2000 in solution mixes with the plasmid DNA and binds to the cells. The cells are allowed to adhere to the well surfaces, become transfected with the plasmid, and express the GPCR at 37° C. for 48 hours. The media on the cells is changed after 16 h.

To assess the ability of the GPCRs to be inhibited by a candidate drug, the drug will be added to each well of the reverse-transfected cells. The cells will also be prepared to contain Fluo-4, which responds to calcium mobilization in response to GPCR activation. A 96-well plate of agonists, each specific to a particular GPCR that corresponds to the same position in the reverse-transfected plate, will be prepared. Using an automatic dispenser, the specific ligand for each GPCR will be added to the reverse-transfected cells expressing the GPCR. In the absence of a drug, the ligand will stimulate the GPCR to signal. In the presence of a drug that binds to that particular GPCR, the signaling is diminished or eliminated. The specificity of the drug for a desired GPCR target can thereby be assessed.

### Example 53

### Provirus Mutation Array

Protease is an HIV-1 protein important for maturation of the virus. Inhibition of protease, for example with protease inhibitor drugs, renders the virus unable to productively infect new cells. In order to map the regions of Protease that are responsible for function, a library of random mutations of the protease gene will be created using the Diversify mutagenesis kit (Clontech), and the mutated protease gene will be cloned back into the context of an HIV-1 Gag-Pol expression plasmid. The expression of Gag is necessary and sufficient for the assembly and budding of virus-like particles, although they are non-infectious. Alternatively, the mutated protease gene can be cloned back into the context of a full HIV-1 provirus (i.e. the full genome of HIV-1 within a plasmid expression vector). Once cloned, individual plasmids of the resulting library will be picked and prepared, each clone containing a defined mutation(s) of protease in the context of a subviral (or viral) expression cassette. Each plasmid will be prepared for reverse transfection and then reverse transfected into HEK-293 cells, as described herein. The function (or nonfunction) of each protease mutation clone is confirmed by harvesting the supernatant for each well, and analyzing it by western blot for Gag using anti-HIV Gag sera. If protease is functional, Gag will be cleaved into several smaller fragments (e.g. Matrix, Capsid, Nucleocapsid). If protease in not functional, Gag will remain uncleaved in a large polyprotein precursor. Wild type protease will be included as a control.

To analyze the effect of protease inhibitors, the same Protease mutation library will be assayed for function in the presence of a protease inhibitor drug. Wild type protease is expected to be inhibited by the protease inhibitor. Other mutations of protease, however, may or may not be affected by the drug due to the presence of drug-resistance mutations. Such mutations would be identified by the function of protease (as detected by western blot cleavage of Gag) in the absence and in the presence of protease inhibitor.

One skilled in the art would recognize that additional proteins (e.g. Integrase, Reverse Transcriptase, Envelope), additional drugs (against different protein targets), and genes from different pathogenic organisms (e.g. Herpes virus, Hepatitis C, Dengue virus, influenza, anthrax) could also be analyzed in a similar manner.

### Example 54

### Mapping of Protein-Protein Interaction Regions

Potassium ion (K⁺) channels have been particularly well studied due to their primary importance in excitable cells (see (Deutsch (2002), Annu Rev Physiol, 64:19-46, Ford, et al. (2002), Prog Drug Res, 58:133-168)). A number of ion channels are known to be regulated by other cellular factors. The KCNQ2 and KCNQ3 voltage-regulated K⁺ channels form a hetero-oligomer responsible for the “M current” found in many neurons (Wang, et al. (1998), Science, 282:1890-3). KCNQ2/3 can be regulated by the muscarinic acetylcholine receptor (mAChR). Three mutation libraries, of KCNQ2, KCNQ3, and mAChR, will each be constructed and reverse transfected into HEK-293 cells in conjunction with the wild type versions of the other two components. The function of each mutation will then be assessed using an ion channel activation assay. Briefly, cells will be loaded with the membrane potential-sensitive dye FMP (1× concentration, Molecular Devices) and then stimulated to assess ion channel function. To assess KCNQ2/3 ion channel function, the cells are stimulated with K+ (addition of K2SO4 to a final concentration of 30 mM). Variants that retain voltage-regulated functionality will open in response to depolarization and a change in fluorescence of FMP dye (ex530/em565) will occur. To assess the function of mAChR, acetylcholine is added. Variants that retain their ability to regulate KCNQ2/3 will alter the response of KCNQ2/3 to K⁺. Thus, in the final assay for each mutant, acetylcholine is added first, then K2SO4 is injected and the signal from FMP dye (ex530/em565) is measured in real-time. Effects of mutations on AChR, KCNQ2, and KCNQ3 will be assessed relative to their wild type functions.

### Example 55

### Receptor Polymorphism Screening

Cell arrays are used to express polymorphic proteins that can be used to screen compounds for selective activity. Splice variants of the growth hormone releasing hormone (GHRH) receptor have been implicated in the pathogenesis of breast and adrenocortical tumors (Freddi, et al. (2005), Clin Endocrinol (Oxf), 62:533-8). Four plasmids, each containing a splice variant of the GHRH receptor (SV1-SV4) are constructed using conventional techniques. Plasmids are arrayed and reverse transfected into HEK-293 cells, as outlined herein. After 24 h, GHRH is added to each well, and binding is detected using a fluorescently-labeled antibody against GHRH.

### Example 56

### Expression of Multiple Proteins by Reverse Transfection

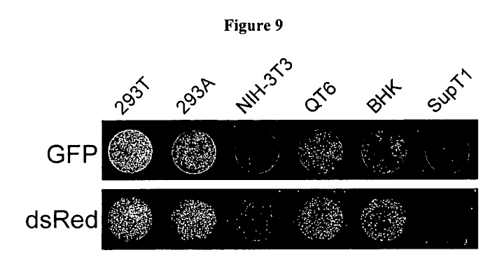

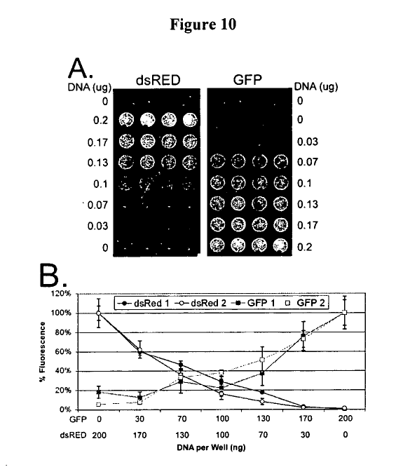

To demonstrate the suitability of the reverse-transfection methodology for high-throughput screening of live cells, 96-well microplates were prepared for the reverse-transfection of a fluorescent protein (a CCR5-GFP fusion protein). 120 ng of the CCR5-GFP eukaryotic expression vector was mixed with 0.3 ul Lipofectamine 2000 and 1% sucrose in 30 ul of Optimem medium, and deposited onto the surface of each well. The plasmid-lipid complex was stored frozen in the well at −20° C. When ready for use, the plates were thawed at 37° C. for 30 min, and 5×10⁴ HEK-293 cells were added to each well in 100 ul of media. The cells were allowed to adhere to the well surfaces, become transfected with the plasmid, and express CCR5-GFP at 37° C. for 24 hours. Protein expression within living transfected cells was visualized by imaging for green fluorescence (FIG. 9A), indicating the successful expression of GFP in 70-90% of the cells per well. Control wells that did not receive plasmid (bottom three wells of FIG. 9A) contained the same number of cells, but were not fluorescent. A similar experiment was repeated for other proteins, in quadruplicate (FIG. 9B). Two of the proteins could be visualized directly (CXCR4-GFP, CCR5-GFP, dsRED) while others were detected by immunofluorescent staining using a V5 epitope tag and a Cy3 secondary antibody (MN Env, CXCR4, CD4). Our results demonstrate that plasmid DNA can be arrayed, reverse-transfected, and expressed in a format that will permit the rapid analysis of libraries of plasmids. A number of cell types, including HEK-293T, HEK-293A, QT6, BHK, HeLa, CF2TH, and NIH-3T3 cell lines have been reverse-transfected using this and other reverse-transfection techniques (FIG. 10 and data not shown).

### Example 57

### Measuring the Variability of Reverse Transfection

The variability of the reverse transfection process was assessed. A plasmid containing GFP reporter, and a plasmid containing dsRED reporter were prepared, and co-arrayed in quadruplicate in microplate wells, one of each plasmid per well. Each plasmid was arrayed at a range of concentrations (0, 30, 70, 100, 130, 170, 200 ng) and matched with a concentration of the co-transfectant such that the total DNA concentration in each well remained constant at 0.2 ug. Two plates were similarly prepared on two separate days. Fluorescence was measured, with red and green filters, using an AlphaArray 7000i and AlphaEase software (FIG. 11). The results between days differed by an average of 7.6% (±5.4% standard deviation), and the coefficient of variation of quadruplicates at each point was an average of 5.9%. The coefficient of variation across an entire 96-well plate of reverse-transfected cells was 5-10% (not shown), typical for a cell-based assay.

### Example 58

### Creation of an Arrayed Library of Env Mutants

Cell arrays were used to produce viral particles expressing a library of Env mutants. Random mutations were introduced into the ectodomains of Env proteins from two HIV-1 strains: MN (Clade B, X4) and DU 151 (Clade C, R5). Parental Env cDNA templates were synthesized to contain an epitope tag (V5) on the C-terminus that was excluded from the mutagenesis process (using unique restriction sites), but which was expressed with each mutated cDNA. Template DNA (˜0.02 ng/ul) was amplified by error-prone PCR using standard components, and concentrations of divalent cations (Mn⁺² and Mg⁺²) that favor the misincorporation of nucleotides (Diversify™ PCR Random Mutagenesis kit, Clontech). Mutations were introduced within a 1.8 kb region of DU 151 Env and a 1.1 kb region of MN Env. The resulting PCR products were cloned into separate expression vectors, each incorporating a CMV promoter, and used to transform E. coli to create bacterial libraries of mutated HIV-1 Env clones, each containing approximately 2.3×10⁴ to 3.8×10⁵ independent clones. Sequencing of clones from each of these (and other) libraries demonstrated that precise mutation rates can be controlled within the boundaries of 0.95 to 12.3 bp/kb. Libraries featured mutation rates of approximately 2.5 (MN) and 5.7 (DU151) bp/clone. Restriction digests, PCR validation, and direct sequencing indicated that 95-98% of transformed bacteria contained inserts.

### Example 59

### Infection and Neutralization Assays can be Performed using Arrays of HIV-1 Env

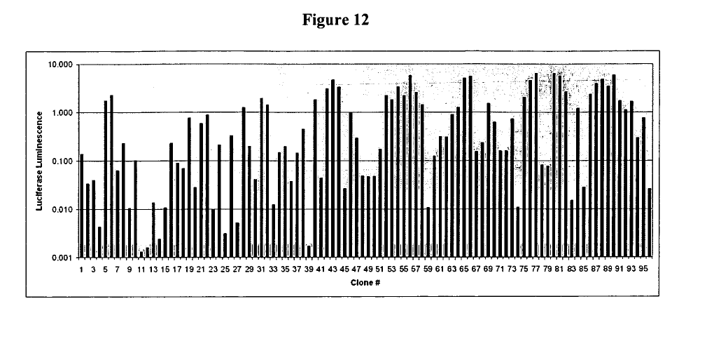

HIV-1 luciferase reporter viruses were prepared from an Env cell array and used in infection and neutralization assays. Plasmids containing different DU151 Env mutants were prepared and arrayed, frozen, in microplate wells as outlined herein. Each array spot also contained a plasmid expressing HIV-1 Gag-Pol, and a plasmid containing CMV promoter-driven renilla-luciferase linked to the HIV packaging signal (Follenzi, et al. (2000), Nat Genet, 25:217-22). These three plasmids were reverse transfected into HEK-293T cells, as described previously, to produce infectious but replication-incompetent reporter viruses each expressing a unique Env variant from the DU151 library. After 48 h, viral particles were harvested from the supernatant of each well and transferred to wells containing target U87 cells stably expressing CD4 and CCR5, in the presence or absence of the neutralizing antibody 4E10. U87 cells were cultured for 48 h, after which cells were lysed, and luciferase reporter activity within each well was measured (Promega Renilla Luciferase Assay System) using a Wallac Victor2V luminometer (FIG. 12). Target cells containing an alternate co-receptor (CD4+ CXCR4, not recognized by DU151) and cells without CD4 (CCR5 alone) were used as controls. Replicate infection of CD4+ CCR5 cells, neutralization by 4E10, and the effects of alternate HIV-1 receptor configurations are shown (FIG. 12B). Detection of the V5 epitope tag was included as an indication of full-length translation of each variant. Details for selected clones are shown in FIGS. 12C and 12D. Successful infection of U87 cells resulted in robust positive signals (1,000 relative light units, RLU) compared to controls and viruses bearing non-function Env mutants (background of 100-200 RLU). Inclusion of 4E10 reduced infection approximately 10-fold compared to infection in the absence of antibody. Duplicate measurements resulted in nearly identical results. Some clones displayed particularly interesting phenotypes. Clone A08 reacted at nearly wild type levels with V5, but did not mediate infection with target cells, suggesting that it either did not reach the cell surface or is functionally disabled (see FIGS. 12C and 12D).

### Example 60

### Removal of Immunodominant Decoy Epitopes in Variable Loops of gp120

Structurally mature gp160 variants are screened to identify those with fewer immunodominant strain-specific variable loop epitopes. A combinatorial library, comprising 161 deletions of various lengths in the V1, V2, and V3 loops, is constructed. Briefly, each loop of the group M consensus gp160 is PCR-amplified in two parts, using one oligo at each end of the Env gene and a series of sense and antisense oligos in the interior of each loop (for each loop there are two non-overlapping PCR reactions amplifying in opposite directions). The interior oligonucleotides contain a conserved 9-nucleotide (in-frame) sequence comprising a NotI site. The PCR products from each half-loop amplification are purified, and then joined by NotI digestion and ligation with DNA ligase (the 5′ half of the library are de-phosphorylated, and ligation products size-selected to ensure assembly of a full-length Env). Ligation at a common restriction site (NotI) results in a library of full-length Env genes bearing deletions in the V1, V2, or V3 loops. The expected number of different possible deletion mutants in each library is 16 for V1 (2 series of 4 oligos), 64 for V2 (2 series of 8 oligos), and 81 for V3 (2 series of 9 oligos). Each deletion ranges in size from 2 amino acids to the entire loop, leaves the flanking Cys residues intact, and inserts a three-Ala residue linker at the site of the deletion (defined by the NotI restriction site) to introduce conformational flexibility in the deleted region. Finally, the deletions are randomly combined with each other by StEP chimeric recombination (as described herein).

This library is screened by cell-surface co-immunofluorescence (two-color) staining of gp160 on the cell surface using bl2 MAb and a polyclonal rabbit serum raised against V1, V2, and V3 loop peptides. Desirable selection criteria are: 1) presence on the cell surface, 2) reactivity with b12, and 3) minimal reactivity with variable loop antiserum. Clones meeting these criteria are assessed for their ability to mediate viral infectivity, and for their susceptibility to bl2 and 4E10 neutralization (as described herein).

### Example 61

### Recombine Env Immunogens for Iterative Screening

In order to incorporate multiple simultaneous mutations, a new Env Array is created that combines the best mutations identified from previous Env Mutation Array screens. The library is generated by first creating chimeras, via genetic shuffling, between a previously immunofocused set of clones (to create clones that combine the mutations identified in first-round screening) and then randomly mutating these chimeras (to introduce additional complexity). The staggered extension process (StEP) is used to create chimeric clones (Zhao, et al. (1998), Nat Biotechnol, 16:258-61). Briefly, the plasmid DNA of selected clones is mixed at equimolar ratios and ˜0.2 pmol used as a heterogeneous template for PCR amplification using primers flanking unique restriction sites engineered within gp160, and standard buffer components. Thermal cycling conditions are: 1 minute at 95° C., followed by 80 cycles of 30 seconds at 94° C. and 15 seconds at 55° C. Amplification products are gel-purified, digested with restriction enzymes to liberate unique cohesive ends, and ligated into the similarly-digested parental vector. The ligated DNA is transformed into E. coli. Additional diversity is introduced by amplifying these chimeric clones using error-prone PCR (Leung, et al. (1989), Technique, 1:11-15).

The resulting Env Array contains 1,850 gp160 variants, each of which contains approximately 4-7 amino acid changes, some of which are random and some of which have already been selected for enhanced neutralization sensitivity. This second-generation Env Array is assessed for gp160 expression and neutralizing antibody (NAb) neutralization. It is possible to use a single library to select for both b12 and 4E10 neutralization because their epitopes do not overlap (neutralization with each NAb is still performed separately). The most promising immunogens (top 1%) from the second generation library are characterized. Previous immunofocusing efforts have isolated variant Env mutants with improvements of 3-30 fold with single point mutations (Pantophlet, et al. (2003), J Virol, 77:642-58, Zwick, et al. (2005), J Virol, 79:1252-61), so substantial increases in neutralization sensitivity are feasible using this strategy. Additional rounds of chimeric recombination, mutation, and screening are conducted to isolate immunogens with 2-3 orders of magnitude greater sensitivity to neutralization by b12 and 4E10 neutralization. Approximately 1-2 rounds of iteration are expected. 1-2 amino acid changes can dramatically influence Env structure and/or function, which would be identified in the first-round library screen (i.e. without iteration). In other cases, combinations of mutations are necessary, which would be identified in the first iteration (chimeric recombination and random mutation will introduce approximately 4-7 amino acid changes per clone).

### Example 62

### Iterative Derivation of Improved Immunogens: Immunofocusing

The mutation array is used to screen HIV-1 gp160 mutants in an iterative mutation/selection process to derive variants in which conserved antigenic sites are exposed, and strain-specific epitopes are eliminated. A library of randomly mutated HIV-1 gp160 (clade C strain DU151) is constructed. The parental gp160 is engineered to contain the predicted consensus binding epitopes for the MAbs b12 and 4E10 (Pantophlet, et al. (2003), J Virol, 77:642-58, Zwick, et al. (2005), J Virol, 79:1252-61). A codon optimized version is used to render expression of the protein Rev-independent, to maximize protein expression, and to facilitate the introduction of convenient restriction sites (Haas, et al. (1996), Current Biology, 6:315-324). The resulting library collectively comprises approximately 4,625 amino acid substitutions, located randomly throughout the 617 amino acid ectodomain of Env (approximately 2.5 amino acid substitutions per clone). A mutation array is constructed from this library and expressed in HEK-293 cells grown in wells, as outlined herein. Each array spot also contains a plasmid expressing HIV-1 Gag-Pol, and a plasmid containing CMV promoter-driven renilla-luciferase linked to the HIV packaging signal (Follenzi, et al. (2000), Nat Genet, 25:217-22). Viral particles harvested from each culture medium supernatant therefore express a unique Env mutation and carry a functional luciferase gene that, if inserted into a host cell genome, is expressed and “reports” infection. Each gp160 mutant clone is screened in neutralization assays using U87 cells stably expressing CD4 and CCR5 in the presence of either b12 or 4E10 at concentrations that span the IC₅₀ and IC₉₀ levels for the parental gp160. Cell infection is monitored by measuring luciferase activity in lysates of target cells within each well two days later (Promega Renilla Luciferase Assay System) using a Wallac Victor2V luminometer. Virus infection is expected to be inversely proportional to the MAb affinity of the mutant gp160 clones. Binding properties of each mutant showing improved MAb affinity are further characterized using limiting dilutions of the relevant MAb to derive its IC₅₀ and IC₉₀.

Chimeras combining the sequences of gp160 mutants with improved binding characteristics for b12 and/or 4E10 serve as parental sequences in further rounds of mutation and screening, to iteratively select for improved immunogenic gp160 variants. Chimeric clones are created using the staggered extension process (StEP) (Zhao, et al. (1998), Nat Biotechnol, 16:258-61). Briefly, the plasmid DNA of selected clones is mixed at equimolar ratios and ˜0.2 pmol used as a heterogeneous template for PCR amplification using primers flanking unique restriction sites engineered within gp160, and standard buffer components. Thermal cycling conditions are: 1 minute at 95° C., followed by 80 cycles of 30 seconds at 94° C. and 15 seconds at 55° C. Amplification products are gel-purified, cloned into expression vectors, and used to transform E. coli by conventional means. Recombination of selected mutations is confirmed by sequencing these clones. Additional diversity can be introduced by amplifying these chimeric clones using error-prone PCR (Leung, et al. (1989), Technique, 1:11-15).

### Example 63

### Identification of CCR5 Residues Responsible for HIV Coreceptor Function and Drug Inhibition

The CCR5 mutation array is used to identify residues that confer drug-resistant co-receptor function for HIV-1. A library of mutant CCR5 cDNA is prepared and arrayed in microplate wells as described herein. Infection assays, as previously described, are performed in the presence of TAK-779 to determine the ability of each CCR5 mutant to act as a co-receptor for HIV-1 strains TNR32 (TAK-779 susceptible) and TNR36 (TAK-779 resistant). Briefly, the CCR5 mutation array is expressed, as described previously, in HEK-293 cells stably expressing CD4. TNR32 and TNR36 HIV-1 reporter viruses containing a luciferase reporter gene are produced as described previously (Baik, et al. (1999), Virology, 259:267-273, Doranz, et al. (1997), J. Exp. Med., 186:1395-1400) and added to each well in the presence or absence of TAK-779. After 48 hours of culture, cells are lysed, and luciferase activity (indicative of cell infection, and therefore preservation of co-receptor function) measured in each well using a Wallac Victor2V. One skilled in the art would recognize that many additional HIV-1, HIV-2, and SIV strains are available for testing, many of which interact with CCR5 differently or use closely-related chemokine receptors as alternate coreceptors.

### Example 64

### Multi-Characteristic Analysis of Data Collected from Mutation Arrays

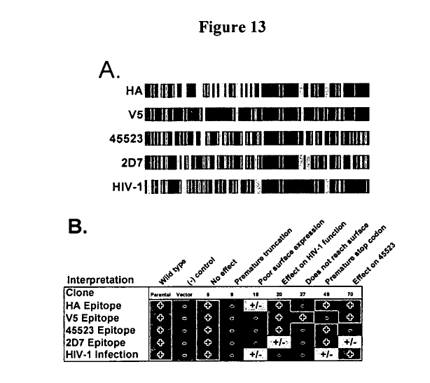

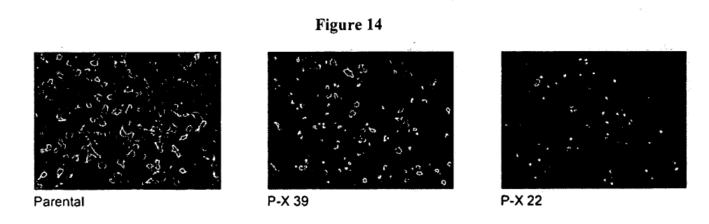

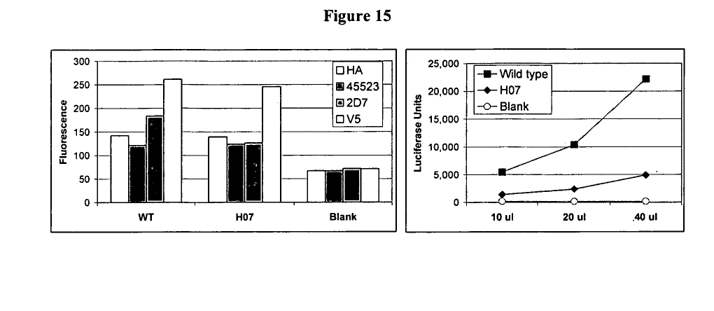

Data collected from multiple structure-activity screens using cell arrays was analyzed to identify residues of importance to protein function. A library of CCR5 mutant cDNA was prepared, arrayed, expressed in HEK-293 cells, and analyzed for surface expression (anti-HA antibody binding), full translation (anti-V5 antibody binding), and monoclonal antibody binding (2D7 and 45523) (FIG. 8). Separately, a library of CCR5 mutant cDNAs was prepared, arrayed, expressed in HEK-293 cells, and analyzed for and HIV-1 co-receptor function (FIG. 13), as described herein. The library included controls without tags and chimeric CCR5/CCR2 clones of defined specificity (Rucker, et al. (1996), Cell, 87:437-446)=. Results for each screen were assigned a color code to indicate the activity of the mutant in comparison with wild-type CCR5: >40% (green), 10-40% (yellow) or <10% (red) (FIG. 14). For each activity, the entire library of mutants was expressed as a bar-code, with each vertical bar representing a single mutant clone, colored according to the activity code. The bar code shown represents the results from one 96-well plate of CCR5 mutants and controls. Other microplates were also tested with similar results. A number of clones that did not support 2D7 binding differed from those that did not support 45523 binding or HIV coreceptor function. A mutant clone that affects the 45523 MAb epitope, did not influence surface expression or HIV-1 coreceptor activity. In contrast, another mutant clone eliminated HIV-1 coreceptor activity, but did not affect the ability of MAb 45523 to bind (FIG. 14). Other CCR5 variants identified in similar assays differed in trafficking, internalization, and/or subcellular localization, as determined by fluorescent microscopy (FIG. 15).

### Example 65

### Characterization of Individual Clones from First Round Screening

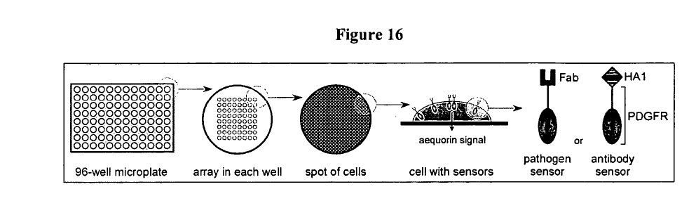

As a result of screening a CCR5 Mutation Array, clone H07 was identified for further examination. H07 displayed wild type surface expression (HA), full-length translation (V5), and wild type reactivity with conformation-specific antibodies 2D7 and 45523, but markedly decreased ability to function as an HIV-1 coreceptor (approximately 20% of wild type activity using the HIV-1 strain DU 151) (FIG. 16). Subsequent sequencing of this clone revealed three mutations. Two of the mutations were silent mutations that resulted in no amino acid change. The third mutation resulted in an amino acid change of Tyr-14 to Cys (Y14C). Examination of the literature revealed that this residue plays a particularly important role in HIV infection because it is sulfated (Farzan, et al. (1999), Cell, 96:667-76). The identification of such a mutation by random screening of CCR5 mutations validates the capability of the mutation array to identify relevant mutations. Further analysis can include site-specific random mutagenesis at Tyr-14 in order to test the complete range of amino acid side chains that are acceptable at that position.

### Example 66

### Expressed, Self-Assembling Protein Sensors for Pathogen Detection

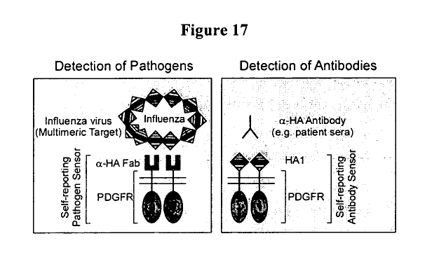

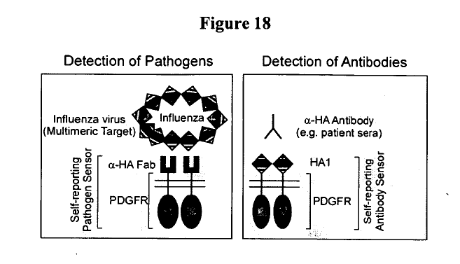

Arrays of expressed, self-assembling protein sensors, capable of binding pathogen antigens are constructed (FIG. 17). Plasmids coding for modular components of a recombinant binding protein for influenza A HA are created (FIG. 18). A single heavy chain Fab cDNA is constructed from the heavy chain variable domain (VH) of the anti-HA monoclonal antibody H36-4 (GenBank Accession # M12236, (Clarke, et al. (1985), J Exp Med, 161:687-704, Mozdzanowska, et al. (2003), J Virol, 77:8322-8)) to which a murine VH signal sequence is added, and the first constant domain (CH1) of the human heavy chain γ1 cDNA (Accession no. AAH69016, amino acid residues 142 to 248). The sequence is modified to incorporate an NheI restriction site at the 5′ end, and codons 530 to 535 of platelet-derived growth factor receptor β (PDGFR-β) at its 3′ end. Overlap PCR is used to fuse this Fab cDNA to a gene fragment for the transmembrane region of PDGFR-β (codons 530 to 1106), introducing a HindIII restriction site at its 3′ end. The resulting chimeric gene (from 5′ to 3′: NheI-VH/CH1-PDGFRβ₅₃₀₋₁₁₀₆-HindIII) is cloned into pcDNA3.1+ (Invitrogen). Simultaneously, the light chain cDNA variable domain (VL) of antibody H36-4 (Accession no. U01973), containing an asymmetric SfiI site at the 3′ end of its complementarity-determining region, is synthesized with a human κ light chain constant domain (CL; Accession no. AJ010446) and cloned into a second pcDNA3.1+plasmid using NheI and HindIII sites. When co-expressed within a cell, the heavy chain and light chain self-assemble to form a chimeric transmembrane protein with receptor specificity for the influenza A HA protein.

The recombinant pathogen sensor is arrayed and expressed in HEK-293 cells as outlined herein. Following expression for 24-48 h, cells are fixed (0.5% paraformaldehyde) and stained with a Cy5-labeled anti-light chain antibody to confirm expression of the sensor. Fluorescence is measured using an AlphaArray 7000i imager. Sensor expression is also verified by western blot using HRP-conjugated antibodies and by immunofluorescence with methanol fixation (for total protein expression).

The sensor is designed so that its target specificity can be changed simply by inserting different complementarity-determining regions (CDRs) from other monoclonal antibodies into the sensor using compatible SfiI sites (Han, et al. (1988), Nucleic Acids Res, 16:11837.). Different MAb DNA sequences are available in GenBank and the Kabat antibody database (Martin (1996), Proteins, 25:130-3). Alternative design strategies for the self-assembling sensor include fusion of the PDGFR intracellular domain with a single-chain heavy and light chain fragment (scFv), and the use of alternative single-transmembrane proteins for signaling and membrane anchoring

### Example 67

### Construction of a Sensor for Antibody Detection

Substituting the target recognition domain of the self-assembling pathogen sensor described herein with proteins other than antibodies enables the detection of targets other than pathogen antigens (FIG. 18). In this case, an antigen, influenza A HA1, is used as the sensor's recognition domain. The resulting sensor is a recombinant binding protein that can recognize anti-HA antibodies. Briefly, cDNA coding for the HA1 ectodomain (residues 51-278 fused with a synthetic leader sequence) is created by DNA synthesis, introducing an NheI restriction site at its 5′ end and including the PDGFR-β codons 530 to 535 at its 3′ end. The resulting DNA fragment is assembled, via overlap PCR, with the PDGFR-β gene transmembrane fragment encoding codons 530 to 1106.

The recombinant antibody sensor is arrayed and expressed in HEK-293 cells as described herein. Following expression for 24-48 h, cells are fixed (0.5% paraformaldehyde) and stained with a Cy3-labeled anti-HA1 antibody (12CA5) to confirm sensor expression. Alternative antibody-specific sensors could include other portions of HA (e.g. residues 15-325), focused regions (e.g. MAb 12CA5 epitope YPYDVPDYA), different strains of influenza (e.g. H3), antigens from other pathogens (e.g. HIV-1 Env), or antigens from other proteins.

### Example 68

### Expressed, Self-Reporting Protein Receptor Arrays

A self-reporting system is developed for arrayed, self-reporting molecular sensors. Self-assembling pathogen sensors (specific for influenza A HA1) and self-assembling antibody sensors (specific for anti-12CA5) are constructed and arrayed, as described herein (FIG. 18). Homodimerization of these sensor proteins upon binding their respective targets activates the intracellular PDGFR domains and induce cellular Ca²⁺-signaling. Arrays are expressed in HEK-293 cells stably expressing the Ca²⁺-sensitive reporter aequorin (HEK-293 aequorin reporter cells). After growth in culture for 24-48 hours, the substrate (coelenterazine) is added to each well, and 60 minutes later molecular targets are added and luminescence monitored in real time. Targets include influenza A virions, dimeric HA-IgG protein, anti-HA monoclonal antibody (12CA5), and polyclonal serum containing anti-HA antibodies. Negative controls include irrelevant virions (dengue, West Nile virus, vaccinia HIV, and MLV particles), irrelevant antibodies, and denatured (heat-destroyed) targets. Cells without sensors also serve as negative controls. Positive controls include anti-light chain antibodies. Detection of targets is measured without additives, in the presence of 10% serum, and in the presence of 100% serum.

Other features for internal validation include duplicate spots of each sensor, multi-epitope sensors that must simultaneously bind independent epitopes to signal, and multiple sensors that recognize independent antigens of the same pathogen. The inclusion of antibodies that recognize different epitopes on the same protein also enables binding of monovalent targets.

### Example 69

### Sensors that Self-Report using Resonant Energy Transfer

Self-assembling sensors for antibodies or antigens, capable of self-reporting target binding in the absence of cell modifications, are constructed, arrayed, and expressed. Modular recombinant binding proteins for influenza A HA and anti-HA antibody are constructed as outlined herein. The sequence for monomeric CFP or YFP is included at the 3′-end of the coding sequence for PDGFR (i.e. PDGFR-CFP and PDGFR-YFP) using standard cloning techniques. Sensors are arrayed and expressed in HEK-293 cells using methods described herein. Cells simultaneously express both fluorescent protein fusion proteins (recognition domain-PDGFR-CFP and recognition domain-PDGFR-YFP) in order for FRET to occur upon target binding and sensor dimerization. The presence of the recombinant sensor proteins in cells is verified by Western blot using anti-light chain or anti-HA secondary antibodies. Screening of targets (influenza A virions, dimeric HA-IgG protein, anti-HA monoclonal antibody (12CA5), and polyclonal serum containing anti-HA antibodies), positive controls (anti-light chain antibodies) and negative controls (irrelevant virions, irrelevant antibodies, and heat-denatured targets), by the array is performed as described herein. One skilled in the art would recognize that additional methods of creating self-reporting sensors, including other fusion proteins for use in FRET, BRET, or protein complementation (Galarneau, et al. (2002), Nat Biotechnol, 20:619-22, Luker, et al. (2004), Methods Enzymol, 385:349-60), are possible.

### Example 70

### Self-Assembling Protein Arrays

In order to assemble a protein array, a 1×3 inch streptavidin-coated glass slide is spotted with plasmids encoding a cDNA library. 120 ng of each purified plasmid expression vector is mixed with 0.3 μl Lipofectamine 2000 and 1% sucrose in 30 ul of Optimem, and spotted onto the surface of each slide with microarrayer pins. After air-drying of the plasmid-lipid complex to the surface, the slide is stored at −20° C. The library is constructed so that each translated protein contains a C-terminal nucleotide-encoded biotin tag (Boutell, et al. (2004), Proteomics, 4:1950-8). When ready for use, 5×10⁴ HEK-293 cells are added onto the slide in 3 ml of media. The cells are allowed to adhere to the surface, become transfected with the plasmid, and express the proteins at 37° C. for 24 hours. Proteins that are secreted from the cells stick to the area around the cell via their C-terminal tag. The cells are lysed with 0.5% Triton X-100 and intracellular proteins are also allowed to attach to the slide surface. Remaining cellular proteins that do not contain the appropriate tag are washed away.

### Example 71

### Spotted Protein Arrays

In order to assemble a protein array, a microplate is prepared for reverse-transfection with plasmids encoding a cDNA library (one plasmid per well). The library of plasmid DNA (0.15 ug per well) is mixed with lipofectamine 2000 and frozen, as described. The library is constructed so that each translated protein contains a C-terminal nucleotide-encoded biotin tag (Boutell, et al. (2004), Proteomics, 4:1950-8). When desired, HEK-293 cells are added. The cells are reverse-transfected and express the protein encoded by the cDNA in each well. After 48 h, the cells are lysed with 0.5% Triton X-100. The lysate is then spotted, using a microarray pin-spotter, on streptavidin-coated glass slides. The proteins are allowed to adhere and excess protein is washed away. The spots will contain biotinylated protein specifically adhered to the location in the array. Additional spotting conditions and surfaces may also be used to ensure that the proteins do not dry or denature.

What is claimed is:

1. A microwell plate comprising an array of nucleic acid molecules and a transfection reagent wherein said nucleic acid molecules and transfection reagent are frozen in solution.

2. The microwell plate of claim 1, wherein the nucleic acid is a plasmid.

3. The microwell plate of claim 1, wherein the nucleic acid is a virus.

4. The microwell plate of claim 1, wherein said array is a microplate.

5. The microwell plate of claim 1, wherein said array is a slide.

6. The microwell plate of claim 1, wherein said solution comprises a sugar.

7. The microwell plate of claim 1, wherein said solution comprises EDTA.

8. An array comprising an array of nucleic acid molecules and a transfection reagent wherein said array of nucleic acid molecules comprises polymorphisms of a gene and wherein said transfection reagent and nucleic acid molecules are frozen or dried onto a surface.

9. An array comprising an array of nucleic acid molecules and a transfection reagent wherein said nucleic acid molecules encode at least one sensor and, optionally, at least one reporter, wherein said nucleic acid molecules and transfection reagent are frozen or dried on to a surface.

10. The array of claim 9 wherein said sensor comprises an antibody or antibody-like molecule.

11. A method of preparing a microarray for reverse-transfection, comprising an array of nucleic acid molecules comprising:
a) depositing a composition comprising a nucleic acid molecule and a transfection reagent into a well of a microarray; and
b) freezing said microarray.

12. The method of claim 11, where said composition comprising a nucleic acid molecule comprises a transfection reagent.

13. The method of claim 11, where said composition comprises a nucleic acid molecule that can be electroporated into a cell.

14. The method of claim 11, wherein said nucleic acid molecule encodes at least part of a virus.

15. The method of claim 11, wherein said nucleic acid molecule encodes for a protein or a transcription element.

16. The method of claim 15 wherein said nucleic acid molecule comprises a promoter and is operatively linked to a reporter gene.

17. The method of claim 15 wherein said nucleic acid molecule encoding for said protein is mutated.

18. The method of claim 15 wherein said protein is fixed onto a surface.

19. The method of claim 15 wherein said protein is specifically attached to a surface using a tag on the protein.

20. The method of claim 11 wherein each well comprising said nucleic acid molecule comprises a specific mutation in said nucleic acid molecule.

21. The method of claim 11 wherein each well or spot of said microarray comprises a nucleic acid molecule comprising a specific cDNA.

22. A method of introducing a nucleic acid molecule into a cell comprising:
a) thawing a microarray comprising a composition comprising a nucleic acid molecule; and
b) contacting a well of said microarray with said cell under appropriate conditions for entry of the nucleic acid molecule into the cell.

23. A method of mapping a function of a nucleic acid molecule product comprising:
a) contacting a composition comprising an array of nucleic acid molecules with a cell under appropriate conditions for entry of the nucleic acid molecule into the cell; and
b) measuring a function of said nucleic acid molecule product.

24. The method of claim 23 wherein the array comprises different mutations of said nucleic acid product and the function of each product is compared to the wild-type product.

25. A method of preparing an array of viable cells in a multi-well plate comprising
a) thawing a frozen microarray comprising an array of nucleic acid molecules; and
b) contacting a well in said microarray with said cells.

26. The method of claim 25 wherein said array of nucleic acid molecules encodes a protein or a transcriptional element.

27. The method of claim 26 wherein said array of nucleic acid molecules comprises mutations of said protein or transcription element.

28. The method of claim 25 wherein said array of nucleic acid molecules encodes different cDNAs encoding for different proteins or transcriptional elements.

29. A frozen microarray comprising an array of cells, a nucleic acid molecule, and a transfection reagent.

30. A frozen microarray comprising a nucleic acid molecules, an array of cells, a transfection reagent, or combinations thereof wherein said nucleic acid molecules encodes at least one sensor and, optionally, at least one reporter.

31. A method of selecting a vaccine candidate protein comprising:
a) contacting a composition comprising an array of nucleic acid molecules which encodes variants of said protein with a cell under appropriate conditions for entry of the nucleic acid molecule into the cell and expression of said protein on the cell surface; and
b) measuring the binding of said protein to a first antibody has desirable binding properties and is specific for said protein and, optionally, to a second antibody, wherein said second antibody is has undesirable binding properties, wherein a protein that binds to the first antibody, but not to the second antibody is selected as a vaccine candidate.

32. A method for detecting a pathogen in a sample comprising:
a) contacting said sample with an array of cells comprising ligands for said pathogens, wherein said ligands comprise a signaling mechanism that is activated upon binding of said pathogens to said ligands; and
b) detecting a signal, whereby detection of said signal indicates the presence of said pathogen.

33. A method for detecting a protein in a sample comprising:
a) contacting said sample with an array of cells comprising binding partners for said protein, wherein said binding partners comprise a signaling mechanism that is activated upon binding of said protein to said binding partners; and
b) detecting a signal, whereby detection of said signal indicates the presence of said protein.
